SEQUENCE LISTING (516 sequences)

SEQ ID NO: 1 - RNA, 19 nt, synthetic construct
  source 1..19: mol_type = other RNA
cttcctggatggcttcaat

SEQ ID NO: 2 - RNA, 19 nt, synthetic construct
  source 1..19: mol_type = other RNA
gtacattaagatggacttc

SEQ ID NO: 3 - RNA, 30 nt, synthetic construct
  source 1..30: mol_type = other RNA
tatctggataggtggtatcaagatctgtaa

SEQ ID NO: 4 - RNA, 25 nt, synthetic construct
  source 1..25: mol_type = other RNA
atgtaactgaaaatgttcttcttta

SEQ ID NO: 5 - RNA, 29 nt, synthetic construct
  source 1..29: mol_type = other RNA
tggataggtggtatcaacattgtaagcac

SEQ ID NO: 6 - RNA, 22 nt, synthetic construct
  source 1..22: mol_type = other RNA
gataggtggtatcaacatctgt

SEQ ID NO: 7 - RNA, 30 nt, synthetic construct
  source 1..30: mol_type = other RNA
tatctggataggtggtatcaacatctgtaa

SEQ ID NO: 8 - RNA, 25 nt, synthetic construct
  source 1..25: mol_type = other RNA
aaacttggaagagtgatgtgatgta

SEQ ID NO: 9 - RNA, 27 nt, synthetic construct
  source 1..27: mol_type = other RNA
gctcacttgttgaggcaaaacttggaa

SEQ ID NO: 10 - RNA, 25 nt, synthetic construct
  source 1..25: mol_type = other RNA
gccttggcaacatttccacttcctg

SEQ ID NO: 11 - RNA, 25 nt, synthetic construct
  source 1..25: mol_type = other RNA
tacacactttacctgttgagaatag

SEQ ID NO: 12 - RNA, 24 nt, synthetic construct
  source 1..24: mol_type = other RNA
gataggtggtatcaacatctgtaa

SEQ ID NO: 13 - RNA, 21 nt, synthetic construct
  source 1..21: mol_type = other RNA
gataggtggtatcaacatctg

SEQ ID NO: 14 - RNA, 25 nt, synthetic construct
  source 1..25: mol_type = other RNA
gataggtggtatcaacatctgtaag

SEQ ID NO: 15 - RNA, 20 nt, synthetic construct
  source 1..20: mol_type = other RNA
ggtggtatcaacatctgtaa

SEQ ID NO: 16 - RNA, 20 nt, synthetic construct
  source 1..20: mol_type = other RNA
gtatcaacatctgtaagcac

SEQ ID NO: 17 - RNA, 30 nt, synthetic construct
  source 1..30: mol_type = other RNA
  misc_difference 27: note = n = a, t, g, or c
cggctaatttcagagggcgctttcttngac

SEQ ID NO: 18 - RNA, 26 nt, synthetic construct
  source 1..26: mol_type = other RNA
acagtggtgctgagatagtataggcc

SEQ ID NO: 19 - RNA, 22 nt, synthetic construct
  source 1..22: mol_type = other RNA
taggccactttgttgctcttgc

SEQ ID NO: 20 - RNA, 19 nt, synthetic construct
  source 1..19: mol_type = other RNA
ttcagagggcgctttcttc

SEQ ID NO: 21 - RNA, 30 nt, synthetic construct
  source 1..30: mol_type = other RNA
tcttcaggtgcaccttctgtttctcaatct

SEQ ID NO: 22 - RNA, 30 nt, synthetic construct
  source 1..30: mol_type = other RNA
tctgtgatactcttcaggtgcaccttctgt

SEQ ID NO: 23 - RNA, 21 nt, synthetic construct
  source 1..21: mol_type = other RNA
tcttctgctcgggaggtgaca

SEQ ID NO: 24 - RNA, 20 nt, synthetic construct
  source 1..20: mol_type = other RNA
ccagttactattcagaagac

SEQ ID NO: 25 - RNA, 20 nt, synthetic construct
  source 1..20: mol_type = other RNA
tcttcaggtgcaccttctgt

SEQ ID NO: 26 - RNA, 18 nt, synthetic construct
  source 1..18: mol_type = other RNA
tgctgctgtcttcttgct

SEQ ID NO: 27 - RNA, 19 nt, synthetic construct
  source 1..19: mol_type = other RNA
ttgttaactttttcccatt

SEQ ID NO: 28 - RNA, 20 nt, synthetic construct
  source 1..20: mol_type = other RNA
tgttaactttttcccattgg

SEQ ID NO: 29 - RNA, 20 nt, synthetic construct
  source 1..20: mol_type = other RNA
cattttgttaactttttccc

SEQ ID NO: 30 - RNA, 19 nt, synthetic construct
  source 1..19: mol_type = other RNA
ctgtagcttcaccctttcc

SEQ ID NO: 31 - RNA, 26 nt, synthetic construct
  source 1..26: mol_type = other RNA
gagagcttcctgtagcttcacccttt

SEQ ID NO: 32 - RNA, 28 nt, synthetic construct
  source 1..28: mol_type = other RNA
tcctgtagcttcaccctttccacaggcg

SEQ ID NO: 33 - RNA, 20 nt, synthetic construct
  source 1..20: mol_type = other RNA
tgtgttacctacccttgtcg

SEQ ID NO: 34 - RNA, 27 nt, synthetic construct
  source 1..27: mol_type = other RNA
tagactatcttttatattctgtaatat

SEQ ID NO: 35 - RNA, 29 nt, synthetic construct
  source 1..29: mol_type = other RNA
gagagcttcctgtagcttcaccctttcca

SEQ ID NO: 36 - RNA, 31 nt, synthetic construct
  source 1..31: mol_type = other RNA
ttcctgtagcttcaccctttccacaggcgtt

SEQ ID NO: 37 - RNA, 23 nt, synthetic construct
  source 1..23: mol_type = other RNA
agcttcctgtagcttcacccttt

SEQ ID NO: 38 - RNA, 29 nt, synthetic construct
  source 1..29: mol_type = other RNA
ggagagagcttcctgtagcttcacccttt

SEQ ID NO: 39 - RNA, 23 nt, synthetic construct
  source 1..23: mol_type = other RNA
gagagcttcctgtagcttcaccc

SEQ ID NO: 40 - RNA, 25 nt, synthetic construct
  source 1..25: mol_type = other RNA
tatgtgttacctacccttgtcggtc

SEQ ID NO: 41 - RNA, 20 nt, synthetic construct
  source 1..20: mol_type = other RNA
ggagagagcttcctgtagct

SEQ ID NO: 42 - RNA, 23 nt, synthetic construct
  source 1..23: mol_type = other RNA
tcaccctttccacaggcgttgca

SEQ ID NO: 43 - RNA, 28 nt, synthetic construct
  source 1..28: mol_type = other RNA
gctgggagagagcttcctgtagcttcac

SEQ ID NO: 44 - RNA, 28 nt, synthetic construct
  source 1..28: mol_type = other RNA
tgttacctacccttgtcggtccttgtac

SEQ ID NO: 45 - RNA, 28 nt, synthetic construct
  source 1..28: mol_type = other RNA
ctgctgtcttcttgctatgaataatgtc

SEQ ID NO: 46 - RNA, 28 nt, synthetic construct
  source 1..28: mol_type = other RNA
ggcgttgcactttgcaatgctgctgtct

SEQ ID NO: 47 - RNA, 28 nt, synthetic construct
  source 1..28: mol_type = other RNA
ttggaaatcaagctgggagagagcttcc

SEQ ID NO: 48 - RNA, 28 nt, synthetic construct
  source 1..28: mol_type = other RNA
ctacccttgtcggtccttgtacattttg

SEQ ID NO: 49 - RNA, 28 nt, synthetic construct
  source 1..28: mol_type = other RNA
gtcaatccgacctgagctttgttgtaga

SEQ ID NO: 50 - RNA, 27 nt, synthetic construct
  source 1..27: mol_type = other RNA
cttgctatgaataatgtcaatccgacc

SEQ ID NO: 51 - RNA, 28 nt, synthetic construct
  source 1..28: mol_type = other RNA
tatatgtgttacctacccttgtcggtcc

SEQ ID NO: 52 - RNA, 20 nt, synthetic construct
  source 1..20: mol_type = other RNA
tttgtgtctttctgagaaac

SEQ ID NO: 53 - RNA, 20 nt, synthetic construct
  source 1..20: mol_type = other RNA
aaagacttaccttaagatac

SEQ ID NO: 54 - RNA, 20 nt, synthetic construct
  source 1..20: mol_type = other RNA
atctgtcaaatcgcctgcag

SEQ ID NO: 55 - RNA, 19 nt, synthetic construct
  source 1..19: mol_type = other RNA
cgccgccatttctcaacag

SEQ ID NO: 56 - RNA, 20 nt, synthetic construct
  source 1..20: mol_type = other RNA
tttgtatttagcatgttccc

SEQ ID NO: 57 - RNA, 17 nt, synthetic construct
  source 1..17: mol_type = other RNA
ccgccatttctcaacag

SEQ ID NO: 58 - RNA, 21 nt, synthetic construct
  source 1..21: mol_type = other RNA
ttctcaggaatttgtgtcttt

SEQ ID NO: 59 - RNA, 11 nt, synthetic construct
  source 1..11: mol_type = other RNA
gacaactcttt

SEQ ID NO: 60 - RNA, 20 nt, synthetic construct
  source 1..20: mol_type = other RNA
tcagcttctgttagccactg

SEQ ID NO: 61 - RNA, 24 nt, synthetic construct
  source 1..24: mol_type = other RNA
tgttcagcttctgttagccactga

SEQ ID NO: 62 - RNA, 27 nt, synthetic construct
  source 1..27: mol_type = other RNA
ctgttcagcttctgttagccactgatt

SEQ ID NO: 63 - RNA, 30 nt, synthetic construct
  source 1..30: mol_type = other RNA
ttctcaacagatctgtcaaatcgcctgcag

SEQ ID NO: 64 - RNA, 25 nt, synthetic construct
  source 1..25: mol_type = other RNA
gccactgattaaatatctttatatc

SEQ ID NO: 65 - RNA, 30 nt, synthetic construct
  source 1..30: mol_type = other RNA
tctgttagccactgattaaatatctttata

SEQ ID NO: 66 - RNA, 31 nt, synthetic construct
  source 1..31: mol_type = other RNA
gagaaactgttcagcttctgttagccactga

SEQ ID NO: 67 - RNA, 31 nt, synthetic construct
  source 1..31: mol_type = other RNA
tctttctgagaaactgttcagcttctgttag

SEQ ID NO: 68 - RNA, 26 nt, synthetic construct
  source 1..26: mol_type = other RNA
cagatctgtcaaatcgcctgcaggta

SEQ ID NO: 69 - RNA, 26 nt, synthetic construct
  source 1..26: mol_type = other RNA
caacagatctgtcaaatcgcctgcag

SEQ ID NO: 70 - RNA, 33 nt, synthetic construct
  source 1..33: mol_type = other RNA
aaactgttcagcttctgttagccactgattaaa

SEQ ID NO: 71 - RNA, 31 nt, synthetic construct
  source 1..31: mol_type = other RNA
gaaactgttcagcttctgttagccactgatt

SEQ ID NO: 72 - RNA, 28 nt, synthetic construct
  source 1..28: mol_type = other RNA
aaactgttcagcttctgttagccactga

SEQ ID NO: 73 - RNA, 28 nt, synthetic construct
  source 1..28: mol_type = other RNA
tgagaaactgttcagcttctgttagcca

SEQ ID NO: 74 - RNA, 32 nt, synthetic construct
  source 1..32: mol_type = other RNA
ttctgagaaactgttcagcttctgttagccac

SEQ ID NO: 75 - RNA, 26 nt, synthetic construct
  source 1..26: mol_type = other RNA
ttctgagaaactgttcagcttctgtt

SEQ ID NO: 76 - RNA, 25 nt, synthetic construct
  source 1..25: mol_type = other RNA
gatctgtcaaatcgcctgcaggtaa

SEQ ID NO: 77 - RNA, 25 nt, synthetic construct
  source 1..25: mol_type = other RNA
ataatgaaaacgccgccatttctca

SEQ ID NO: 78 - RNA, 25 nt, synthetic construct
  source 1..25: mol_type = other RNA
aaactgttcagcttctgttagccac

SEQ ID NO: 79 - RNA, 25 nt, synthetic construct
  source 1..25: mol_type = other RNA
ttgtgtctttctgagaaactgttca

SEQ ID NO: 80 - RNA, 25 nt, synthetic construct
  source 1..25: mol_type = other RNA
ccaattctcaggaatttgtgtcttt

SEQ ID NO: 81 - RNA, 25 nt, synthetic construct
  source 1..25: mol_type = other RNA
atcgcctgcaggtaaaagcatatgg

SEQ ID NO: 82 - RNA, 30 nt, synthetic construct
  source 1..30: mol_type = other RNA
tgaaaacgccgccatttctcaacagatctg

SEQ ID NO: 83 - RNA, 30 nt, synthetic construct
  source 1..30: mol_type = other RNA
cataatgaaaacgccgccatttctcaacag

SEQ ID NO: 84 - RNA, 30 nt, synthetic construct
  source 1..30: mol_type = other RNA
tgttcagcttctgttagccactgattaaat

SEQ ID NO: 85 - RNA, 24 nt, synthetic construct
  source 1..24: mol_type = other RNA
cagatctgtcaaatcgcctgcagg

SEQ ID NO: 86 - RNA, 27 nt, synthetic construct
  source 1..27: mol_type = other RNA
caacagatctgtcaaatcgcctgcagg

SEQ ID NO: 87 - RNA, 29 nt, synthetic construct
  source 1..29: mol_type = other RNA
ctcaacagatctgtcaaatcgcctgcagg

SEQ ID NO: 88 - RNA, 23 nt, synthetic construct
  source 1..23: mol_type = other RNA
gatctgtcaaatcgcctgcaggt

SEQ ID NO: 89 - RNA, 22 nt, synthetic construct
  source 1..22: mol_type = other RNA
gatctgtcaaatcgcctgcagg

SEQ ID NO: 90 - RNA, 21 nt, synthetic construct
  source 1..21: mol_type = other RNA
gatctgtcaaatcgcctgcag

SEQ ID NO: 91 - RNA, 25 nt, synthetic construct
  source 1..25: mol_type = other RNA
cagatctgtcaaatcgcctgcaggt

SEQ ID NO: 92 - RNA, 23 nt, synthetic construct
  source 1..23: mol_type = other RNA
cagatctgtcaaatcgcctgcag

SEQ ID NO: 93 - RNA, 25 nt, synthetic construct
  source 1..25: mol_type = other RNA
gtgtctttctgagaaactgttcagc

SEQ ID NO: 94 - RNA, 28 nt, synthetic construct
  source 1..28: mol_type = other RNA
gagaaactgttcagcttctgttagccac

SEQ ID NO: 95 - RNA, 28 nt, synthetic construct
  source 1..28: mol_type = other RNA
gaaactgttcagcttctgttagccactg

SEQ ID NO: 96 - RNA, 24 nt, synthetic construct
  source 1..24: mol_type = other RNA
ctgttcagcttctgttagccactg

SEQ ID NO: 97 - RNA, 27 nt, synthetic construct
  source 1..27: mol_type = other RNA
atctgtcaaatcgcctgcaggtaaaag

SEQ ID NO: 98 - RNA, 29 nt, synthetic construct
  source 1..29: mol_type = other RNA
gatctgtcaaatcgcctgcaggtaaaagc

SEQ ID NO: 99 - RNA, 19 nt, synthetic construct
  source 1..19: mol_type = other RNA
gctgaattatttcttcccc

SEQ ID NO: 100 - RNA, 19 nt, synthetic construct
  source 1..19: mol_type = other RNA
tttttctgtctgacagctg

SEQ ID NO: 101 - RNA, 18 nt, synthetic construct
  source 1..18: mol_type = other RNA
tctgtttttgaggattgc

SEQ ID NO: 102 - RNA, 17 nt, synthetic construct
  source 1..17: mol_type = other RNA
ccaccgcagattcaggc

SEQ ID NO: 103 - RNA, 17 nt, synthetic construct
  source 1..17: mol_type = other RNA
gcccaatgccatcctgg

SEQ ID NO: 104 - RNA, 17 nt, synthetic construct
  source 1..17: mol_type = other RNA
tttgcagacctcctgcc

SEQ ID NO: 105 - RNA, 17 nt, synthetic construct
  source 1..17: mol_type = other RNA
cagtttgccgctgccca

SEQ ID NO: 106 - RNA, 20 nt, synthetic construct
  source 1..20: mol_type = other RNA
gttgcattcaatgttctgac

SEQ ID NO: 107 - RNA, 21 nt, synthetic construct
  source 1..21: mol_type = other RNA
atttttcctgtagaatactgg

SEQ ID NO: 108 - RNA, 34 nt, synthetic construct
  source 1..34: mol_type = other RNA
gctgcccaatgcgatcctggagttcctgtaagat

SEQ ID NO: 109 - RNA, 28 nt, synthetic construct
  source 1..28: mol_type = other RNA
gctgcccaatgccatcctggagttcctg

SEQ ID NO: 110 - RNA, 31 nt, synthetic construct
  source 1..31: mol_type = other RNA
gctgcccaatgccatcctggagttcctgtaa

SEQ ID NO: 111 - RNA, 31 nt, synthetic construct
  source 1..31: mol_type = other RNA
caatgccatcctggagttcctgtaagatacc

SEQ ID NO: 112 - RNA, 32 nt, synthetic construct
  source 1..32: mol_type = other RNA
gctgcccaatgccatcctggagttcctgtaag

SEQ ID NO: 113 - RNA, 30 nt, synthetic construct
  source 1..30: mol_type = other RNA
ccaatgccatcctggagttcctgtaagata

SEQ ID NO: 114 - RNA, 39 nt, synthetic construct
  source 1..39: mol_type = other RNA
ttgccgctgcccaatgccatcctggagttcctgtaagat

SEQ ID NO: 115 - RNA, 34 nt, synthetic construct
  source 1..34: mol_type = other RNA
gctgcccaatgccatcctggagttcctgtaagat

SEQ ID NO: 116 - RNA, 27 nt, synthetic construct
  source 1..27: mol_type = other RNA
caatgccatcctggagttcctgtaaga

SEQ ID NO: 117 - RNA, 26 nt, synthetic construct
  source 1..26: mol_type = other RNA
cagtttgccgctgcccaatgccatcc

SEQ ID NO: 118 - RNA, 24 nt, synthetic construct
  source 1..24: mol_type = other RNA
cttccccagttgcattcaatgttc

SEQ ID NO: 119 - RNA, 23 nt, synthetic construct
  source 1..23: mol_type = other RNA
ctggcatctgtttttgaggattg

SEQ ID NO: 120 - RNA, 20 nt, synthetic construct
  source 1..20: mol_type = other RNA
ttagatctgtcgccctacct

SEQ ID NO: 121 - RNA, 39 nt, synthetic construct
  source 1..39: mol_type = other RNA
gctgcccaatgccatcctggagttcctgtaagataccaa

SEQ ID NO: 122 - RNA, 34 nt, synthetic construct
  source 1..34: mol_type = other RNA
gcccaatgccatcctggagttcctgtaagatacc

SEQ ID NO: 123 - RNA, 25 nt, synthetic construct
  source 1..25: mol_type = other RNA
catcctggagttcctgtaagatacc

SEQ ID NO: 124 - RNA, 28 nt, synthetic construct
  source 1..28: mol_type = other RNA
tgccatcctggagttcctgtaagatacc

SEQ ID NO: 125 - RNA, 25 nt, synthetic construct
  source 1..25: mol_type = other RNA
tgccatcctggagttcctgtaagat

SEQ ID NO: 126 - RNA, 28 nt, synthetic construct
  source 1..28: mol_type = other RNA
caatgccatcctggagttcctgtaagat

SEQ ID NO: 127 - RNA, 31 nt, synthetic construct
  source 1..31: mol_type = other RNA
gcccaatgccatcctggagttcctgtaagat

SEQ ID NO: 128 - RNA, 28 nt, synthetic construct
  source 1..28: mol_type = other RNA
gcccaatgccatcctggagttcctgtaa

SEQ ID NO: 129 - RNA, 34 nt, synthetic construct
  source 1..34: mol_type = other RNA
gccgctgcccaatgacatcctggagttcctgtaa

SEQ ID NO: 130 - RNA, 25 nt, synthetic construct
  source 1..25: mol_type = other RNA
gccatcctggagttcctgtaagata

SEQ ID NO: 131 - RNA, 25 nt, synthetic construct
  source 1..25: mol_type = other RNA
ccaatgccatcctggagttcctgta

SEQ ID NO: 132 - RNA, 25 nt, synthetic construct
  source 1..25: mol_type = other RNA
ctgacaacagtttgccgctgcccaa

SEQ ID NO: 133 - RNA, 25 nt, synthetic construct
  source 1..25: mol_type = other RNA
tttgaggattgctgaattatttctt

SEQ ID NO: 134 - RNA, 30 nt, synthetic construct
  source 1..30: mol_type = other RNA
cagtttgccgctgcccaatgccatcctgga

SEQ ID NO: 135 - RNA, 30 nt, synthetic construct
  source 1..30: mol_type = other RNA
ttgccgctgcccaatgccatcctggagttc

SEQ ID NO: 136 - RNA, 25 nt, synthetic construct
  source 1..25: mol_type = other RNA
tttgccgctgcccaatgccatcctg

SEQ ID NO: 137 - RNA, 22 nt, synthetic construct
  source 1..22: mol_type = other RNA
ccaatgccatcctggagttcct

SEQ ID NO: 138 - RNA, 29 nt, synthetic construct
  source 1..29: mol_type = other RNA
cccaatgccatcctggagttcctgtaaga

SEQ ID NO: 139 - RNA, 28 nt, synthetic construct
  source 1..28: mol_type = other RNA
ccgctgcccaatgccatcctggagttcc

SEQ ID NO: 140 - RNA, 30 nt, synthetic construct
  source 1..30: mol_type = other RNA
cccaatgccatcctggagttcctgtaagat

SEQ ID NO: 141 - RNA, 30 nt, synthetic construct
  source 1..30: mol_type = other RNA
ccgctgcccaatgccatcctggagttcctg

SEQ ID NO: 142 - RNA, 30 nt, synthetic construct
  source 1..30: mol_type = other RNA
tgcccaatgccatcctggagttcctgtaag

SEQ ID NO: 143 - RNA, 28 nt, synthetic construct
  source 1..28: mol_type = other RNA
cccaatgccatcctggagttcctgtaag

SEQ ID NO: 144 - RNA, 28 nt, synthetic construct
  source 1..28: mol_type = other RNA
tgcccaatgccatcctggagttcctgta

SEQ ID NO: 145 - RNA, 22 nt, synthetic construct
  source 1..22: mol_type = other RNA
caatgccatcctggagttcctg

SEQ ID NO: 146 - RNA, 29 nt, synthetic construct
  source 1..29: mol_type = other RNA
agcctctcgctcactcaccctgcaaagga

SEQ ID NO: 147 - RNA, 29 nt, synthetic construct
  source 1..29: mol_type = other RNA
ccactcagagctcagatcttctaacttcc

SEQ ID NO: 148 - RNA, 27 nt, synthetic construct
  source 1..27: mol_type = other RNA
cttccactcagagctcagatcttctaa

SEQ ID NO: 149 - RNA, 25 nt, synthetic construct
  source 1..25: mol_type = other RNA
gggatccagtatacttacaggctcc

SEQ ID NO: 150 - RNA, 17 nt, synthetic construct
  source 1..17: mol_type = other RNA
ctcagagctcagatctt

SEQ ID NO: 151 - RNA, 15 nt, synthetic construct
  source 1..15: mol_type = other RNA
ggctgctttgccctc

SEQ ID NO: 152 - RNA, 27 nt, synthetic construct
  source 1..27: mol_type = other RNA
ctcagatcttctaacttcctctttaac

SEQ ID NO: 153 - RNA, 29 nt, synthetic construct
  source 1..29: mol_type = other RNA
ctcagagctcagatcttctaacttcctct

SEQ ID NO: 154 - RNA, 27 nt, synthetic construct
  source 1..27: mol_type = other RNA
cgccttccactcagagctcagatcttc

SEQ ID NO: 155 - RNA, 27 nt, synthetic construct
  source 1..27: mol_type = other RNA
tcagctcttgaagtaaacggtttaccg

SEQ ID NO: 156 - RNA, 27 nt, synthetic construct
  source 1..27: mol_type = other RNA
tttgccctcagctcttgaagtaaacgg

SEQ ID NO: 157 - RNA, 27 nt, synthetic construct
  source 1..27: mol_type = other RNA
ggctgctttgccctcagctcttgaagt

SEQ ID NO: 158 - RNA, 26 nt, synthetic construct
  source 1..26: mol_type = other RNA
caggagctaggtcaggctgctttgcc

SEQ ID NO: 159 - RNA, 25 nt, synthetic construct
  source 1..25: mol_type = other RNA
tccaatagtggtcagtccaggagct

SEQ ID NO: 160 - RNA, 27 nt, synthetic construct
  source 1..27: mol_type = other RNA
aaagagaatgggatccagtatacttac

SEQ ID NO: 161 - RNA, 27 nt, synthetic construct
  source 1..27: mol_type = other RNA
aaatagctagagccaaagagaatggga

SEQ ID NO: 162 - RNA, 33 nt, synthetic construct
  source 1..33: mol_type = other RNA
ggctgctttgccctcagctcttgaagtaaacgg

SEQ ID NO: 163 - RNA, 30 nt, synthetic construct
  source 1..30: mol_type = other RNA
aggctgctttgccctcagctcttgaagtaa

SEQ ID NO: 164 - RNA, 30 nt, synthetic construct
  source 1..30: mol_type = other RNA
gtcaggctgctttgccctcagctcttgaag

SEQ ID NO: 165 - RNA, 30 nt, synthetic construct
  source 1..30: mol_type = other RNA
aggtcaggctgctttgccctcagctcttga

SEQ ID NO: 166 - RNA, 25 nt, synthetic construct
  source 1..25: mol_type = other RNA
cagagctcagatcttctaacttcct

SEQ ID NO: 167 - RNA, 25 nt, synthetic construct
  source 1..25: mol_type = other RNA
cttacaggctccaatagtggtcagt

SEQ ID NO: 168 - RNA, 25 nt, synthetic construct
  source 1..25: mol_type = other RNA
atgggatccagtatacttacaggct

SEQ ID NO: 169 - skipped

SEQ ID NO: 170 - RNA, 27 nt, synthetic construct
  source 1..27: mol_type = other RNA
aacttcctctttaacagaaaagcatac

SEQ ID NO: 171 - RNA, 23 nt, synthetic construct
  source 1..23: mol_type = other RNA
ctcataccttctgcttgatgatc

SEQ ID NO: 172 - RNA, 20 nt, synthetic construct
  source 1..20: mol_type = other RNA
tcaaggaagatggcatttct

SEQ ID NO: 173 - RNA, 20 nt, synthetic construct
  source 1..20: mol_type = other RNA
gaaagccagtcggtaagttc

SEQ ID NO: 174 - RNA, 15 nt, synthetic construct
  source 1..15: mol_type = other RNA
cacccaccatcaccc

SEQ ID NO: 175 - RNA, 23 nt, synthetic construct
  source 1..23: mol_type = other RNA
cctctgtgattttataacttgat

SEQ ID NO: 176 - RNA, 20 nt, synthetic construct
  source 1..20: mol_type = other RNA
tgatatcctcaaggtcaccc

SEQ ID NO: 177 - RNA, 30 nt, synthetic construct
  source 1..30: mol_type = other RNA
ggtacctccaacatcaaggaagatggcatt

SEQ ID NO: 178 - RNA, 26 nt, synthetic construct
  source 1..26: mol_type = other RNA
atttctagtttggagatggcagtttc

SEQ ID NO: 179 - RNA, 26 nt, synthetic construct
  source 1..26: mol_type = other RNA
catcaaggaagatggcatttctagtt

SEQ ID NO: 180 - RNA, 26 nt, synthetic construct
  source 1..26: mol_type = other RNA
gagcaggtacctccaacatcaaggaa

SEQ ID NO: 181 - RNA, 30 nt, synthetic construct
  source 1..30: mol_type = other RNA
ctccaacatcaaggaagatggcatttctag

SEQ ID NO: 182 - RNA, 25 nt, synthetic construct
  source 1..25: mol_type = other RNA
accagagtaacagtctgagtaggag

SEQ ID NO: 183 - RNA, 25 nt, synthetic construct
  source 1..25: mol_type = other RNA
caccagagtaacagtctgagtagga

SEQ ID NO: 184 - RNA, 25 nt, synthetic construct
  source 1..25: mol_type = other RNA
tcaccagagtaacagtctgagtagg

SEQ ID NO: 185 - RNA, 25 nt, synthetic construct
  source 1..25: mol_type = other RNA
gtcaccagagtaacagtctgagtag

SEQ ID NO: 186 - RNA, 26 nt, synthetic construct
  source 1..26: mol_type = other RNA
accagagtaacagtctgagtaggagc

SEQ ID NO: 187 - RNA, 24 nt, synthetic construct
  source 1..24: mol_type = other RNA
ttctgtccaagcccggttgaaatc

SEQ ID NO: 188 - RNA, 30 nt, synthetic construct
  source 1..30: mol_type = other RNA
acatcaaggaagatggcatttctagtttgg

SEQ ID NO: 189 - RNA, 25 nt, synthetic construct
  source 1..25: mol_type = other RNA
acatcaaggaagatggcatttctag

SEQ ID NO: 190 - RNA, 25 nt, synthetic construct
  source 1..25: mol_type = other RNA
atcattttttctcataccttctgct

SEQ ID NO: 191 - RNA, 21 nt, synthetic construct
  source 1..21: mol_type = other RNA
cacccaccatcaccctctgtg

SEQ ID NO: 192 - RNA, 22 nt, synthetic construct
  source 1..22: mol_type = other RNA
atcatctcgttgatatcctcaa

SEQ ID NO: 193 - RNA, 28 nt, synthetic construct
  source 1..28: mol_type = other RNA
ctccaacatcaaggaagatggcatttct

SEQ ID NO: 194 - RNA, 25 nt, synthetic construct
  source 1..25: mol_type = other RNA
catcaaggaagatggcatttctagt

SEQ ID NO: 195 - RNA, 18 nt, synthetic construct
  source 1..18: mol_type = other RNA
ttgctggtcttgtttttc

SEQ ID NO: 196 - RNA, 16 nt, synthetic construct
  source 1..16: mol_type = other RNA
ccgtaatgattgttct

SEQ ID NO: 197 - RNA, 18 nt, synthetic construct
  source 1..18: mol_type = other RNA
gctggtcttgtttttcaa

SEQ ID NO: 198 - RNA, 20 nt, synthetic construct
  source 1..20: mol_type = other RNA
tggtcttgtttttcaaattt

SEQ ID NO: 199 - RNA, 20 nt, synthetic construct
  source 1..20: mol_type = other RNA
gtcttgtttttcaaattttg

SEQ ID NO: 200 - RNA, 20 nt, synthetic construct
  source 1..20: mol_type = other RNA
cttgtttttcaaattttggg

SEQ ID NO: 201 - RNA, 19 nt, synthetic construct
  source 1..19: mol_type = other RNA
tgtttttcaaattttgggc

SEQ ID NO: 202 - RNA, 33 nt, synthetic construct
  source 1..33: mol_type = other RNA
tccaactggggacgcctctgttccaaatcctgc

SEQ ID NO: 203 - RNA, 21 nt, synthetic construct
  source 1..21: mol_type = other RNA
tcctgcattgttgcctgtaag

SEQ ID NO: 204 - RNA, 30 nt, synthetic construct
  source 1..30: mol_type = other RNA
tccaactggggacgcctctgttccaaatcc

SEQ ID NO: 205 - RNA, 21 nt, synthetic construct
  source 1..21: mol_type = other RNA
actggggacgcctctgttcca

SEQ ID NO: 206 - RNA, 20 nt, synthetic construct
  source 1..20: mol_type = other RNA
ccgtaatgattgttctagcc

SEQ ID NO: 207 - RNA, 20 nt, synthetic construct
  source 1..20: mol_type = other RNA
tgttaaaaaacttacttcga

SEQ ID NO: 208 - RNA, 18 nt, synthetic construct
  source 1..18: mol_type = other RNA
ctgttgcctccggttctg

SEQ ID NO: 209 - RNA, 18 nt, synthetic construct
  source 1..18: mol_type = other RNA
ttggctctggcctgtcct

SEQ ID NO: 210 - RNA, 33 nt, synthetic construct
  source 1..33: mol_type = other RNA
ttcaactgttgcctccggttctgaaggtgttct

SEQ ID NO: 211 - RNA, 25 nt, synthetic construct
  source 1..25: mol_type = other RNA
tacttcatcccactgattctgaatt

SEQ ID NO: 212 - RNA, 25 nt, synthetic construct
  source 1..25: mol_type = other RNA
ctgaaggtgttcttgtacttcatcc

SEQ ID NO: 213 - RNA, 25 nt, synthetic construct
  source 1..25: mol_type = other RNA
ctgttgcctccggttctgaaggtgt

SEQ ID NO: 214 - RNA, 30 nt, synthetic construct
  source 1..30: mol_type = other RNA
ctgttgcctccggttctgaaggtgttcttg

SEQ ID NO: 215 - RNA, 30 nt, synthetic construct
  source 1..30: mol_type = other RNA
caactgttgcctccggttctgaaggtgttc

SEQ ID NO: 216 - RNA, 30 nt, synthetic construct
  source 1..30: mol_type = other RNA
ttgcctccggttctgaaggtgttcttgtac

SEQ ID NO: 217 - RNA, 25 nt, synthetic construct
  source 1..25: mol_type = other RNA
gttgcctccggttctgaaggtgttc

SEQ ID NO: 218 - RNA, 23 nt, synthetic construct
  source 1..23: mol_type = other RNA
ctccggttctgaaggtgttcttg

SEQ ID NO: 219 - RNA, 22 nt, synthetic construct
  source 1..22: mol_type = other RNA
ctccggttctgaaggtgttctt

SEQ ID NO: 220 - RNA, 21 nt, synthetic construct
  source 1..21: mol_type = other RNA
ctccggttctgaaggtgttct

SEQ ID NO: 221 - RNA, 20 nt, synthetic construct
  source 1..20: mol_type = other RNA
ctccggttctgaaggtgttc

SEQ ID NO: 222 - RNA, 19 nt, synthetic construct
  source 1..19: mol_type = other RNA
ctccggttctgaaggtgtt

SEQ ID NO: 223 - RNA, 25 nt, synthetic construct
  source 1..25: mol_type = other RNA
cattcaactgttgcctccggttctg

SEQ ID NO: 224 - RNA, 24 nt, synthetic construct
  source 1..24: mol_type = other RNA
ctgttgcctccggttctgaaggtg

SEQ ID NO: 225 - RNA, 31 nt, synthetic construct
  source 1..31: mol_type = other RNA
cattcaactgttgcctccggttctgaaggtg

SEQ ID NO: 226 - RNA, 21 nt, synthetic construct
  source 1..21: mol_type = other RNA
tactaaccttggtttctgtga

SEQ ID NO: 227 - RNA, 27 nt, synthetic construct
  source 1..27: mol_type = other RNA
tgtatagggaccctccttccatgactc

SEQ ID NO: 228 - RNA, 25 nt, synthetic construct
  source 1..25: mol_type = other RNA
ctaaccttggtttctgtgattttct

SEQ ID NO: 229 - RNA, 27 nt, synthetic construct
  source 1..27: mol_type = other RNA
ggtatctttgatactaaccttggtttc

SEQ ID NO: 230 - RNA, 22 nt, synthetic construct
  source 1..22: mol_type = other RNA
attctttcaactagaataaaag

SEQ ID NO: 231 - RNA, 25 nt, synthetic construct
  source 1..25: mol_type = other RNA
gattctgaattctttcaactagaat

SEQ ID NO: 232 - RNA, 20 nt, synthetic construct
  source 1..20: mol_type = other RNA
atcccactgattctgaattc

SEQ ID NO: 233 - RNA, 22 nt, synthetic construct
  source 1..22: mol_type = other RNA
aaccgagaccggacaggattct

SEQ ID NO: 234 - RNA, 20 nt, synthetic construct
  source 1..20: mol_type = other RNA
ctgttgcagtaatctatgag

SEQ ID NO: 235 - RNA, 23 nt, synthetic construct
  source 1..23: mol_type = other RNA
tgccattgtttcatcagctcttt

SEQ ID NO: 236 - RNA, 20 nt, synthetic construct
  source 1..20: mol_type = other RNA
tgcagtaatctatgagtttc

SEQ ID NO: 237 - RNA, 20 nt, synthetic construct
  source 1..20: mol_type = other RNA
tcctgtaggacattggcagt

SEQ ID NO: 238 - RNA, 18 nt, synthetic construct
  source 1..18: mol_type = other RNA
gagtcttctaggagcctt

SEQ ID NO: 239 - RNA, 25 nt, synthetic construct
  source 1..25: mol_type = other RNA
ggccaaacctcggcttacctgaaat

SEQ ID NO: 240 - RNA, 20 nt, synthetic construct
  source 1..20: mol_type = other RNA
ggccaaacctcggcttacct

SEQ ID NO: 241 - RNA, 29 nt, synthetic construct
  source 1..29: mol_type = other RNA
aatcagctgggagagagcttcctgtagct

SEQ ID NO: 242 - RNA, 26 nt, synthetic construct
  source 1..26: mol_type = other RNA
tcgttcttctgtcgtcgtaacgtttc

SEQ ID NO: 243 - RNA, 16 nt, synthetic construct
  source 1..16: mol_type = other RNA
caccgattgtcttcga

SEQ ID NO: 244 - RNA, 17 nt, synthetic construct
  source 1..17: mol_type = other RNA
cccttgtacgatttatg

SEQ ID NO: 245 - RNA, 18 nt, synthetic construct
  source 1..18: mol_type = other RNA
tctgtgtttaaggactct

SEQ ID NO: 246 - RNA, 31 nt, synthetic construct
  source 1..31: mol_type = other RNA
gccgctgcccaatgccatcctggagttcctg

SEQ ID NO: 247 - RNA, 29 nt, synthetic construct
  source 1..29: mol_type = other RNA
attagatctgtcgccctacctcttttttc

SEQ ID NO: 248 - RNA, 27 nt, synthetic construct
  source 1..27: mol_type = other RNA
tgtcgccctacctcttttttctgtctg

SEQ ID NO: 249 - RNA, 25 nt, synthetic construct
  source 1..25: mol_type = other RNA
gcccaatgccatcctggagttcctg

SEQ ID NO: 250 - RNA, 30 nt, synthetic construct
  source 1..30: mol_type = other RNA
gagcctctcgctcactcaccctgcaaagga

SEQ ID NO: 251 - RNA, 36 nt, synthetic construct
  source 1..36: mol_type = other RNA
atcattttttctcataccttctgctaggagctaaaa

SEQ ID NO: 252 - RNA, 26 nt, synthetic construct
  source 1..26: mol_type = other RNA
ggaagctaaggaagaagctgagcagg

SEQ ID NO: 253 - RNA, 30 nt, synthetic construct; identical to SEQ ID NO: 113
  source 1..30: mol_type = other RNA

SEQ ID NO: 254 - RNA, 30 nt, synthetic construct
  source 1..30: mol_type = other RNA
gctgcccaatgccatcctggagttcctgta

SEQ ID NO: 255 - RNA, 27 nt, synthetic construct
  source 1..27: mol_type = other RNA
caatgccatcctggagttcctgtaaga

SEQ ID NO: 256 - RNA, 39 nt, synthetic construct; identical to SEQ ID NO: 121
  source 1..39: mol_type = other RNA

SEQ ID NO: 257 - RNA, 30 nt, synthetic construct
  source 1..30: mol_type = other RNA
gcccaatgccatcctggagttcctgtaaga

SEQ ID NO: 258 - RNA, 28 nt, synthetic construct
  source 1..28: mol_type = other RNA
tgccatcctggagttcctgtaagatacc

SEQ ID NO: 259 - RNA, 25 nt, synthetic construct
  source 1..25: mol_type = other RNA
tgccatcctggagttcctgtaagat

SEQ ID NO: 260 - RNA, 28 nt, synthetic construct
  source 1..28: mol_type = other RNA
caatgccatcctggagttcctgtaagat

SEQ ID NO: 261 - RNA, 31 nt, synthetic construct; identical to SEQ ID NO: 127
  source 1..31: mol_type = other RNA

SEQ ID NO: 262 - RNA, 30 nt, synthetic construct; identical to SEQ ID NO: 135
  source 1..30: mol_type = other RNA

SEQ ID NO: 263 - RNA, 28 nt, synthetic construct
  source 1..28: mol_type = other RNA
gcccaatgccatcctggagttcctgtaa

SEQ ID NO: 264 - RNA, 30 nt, synthetic construct
  source 1..30: mol_type = other RNA
gccgctgcccaatgccatcctggagttcct

SEQ ID NO: 265 - RNA, 22 nt, synthetic construct
  source 1..22: mol_type = other RNA
caatgccatcctggagttcctg

SEQ ID NO: 266 - RNA, 25 nt, synthetic construct
  source 1..25: mol_type = other RNA
gcccaatgccatcctggagttcctg

SEQ ID NO: 267 - RNA, 28 nt, synthetic construct
  source 1..28: mol_type = other RNA
gctgcccaatgccatcctggagttcctg

SEQ ID NO: 268 - RNA, 31 nt, synthetic construct; identical to SEQ ID NO: 110
  source 1..31: mol_type = other RNA

SEQ ID NO: 269 - RNA, 31 nt, synthetic construct; identical to SEQ ID NO: 111
  source 1..31: mol_type = other RNA

SEQ ID NO: 270 - DNA, 30 bp, synthetic construct
  source 1..30: mol_type = other DNA
ttgccgctgcccaatgccatcctggagttc

SEQ ID NO: 271 - DNA, 30 bp, synthetic construct
  source 1..30: mol_type = other DNA
cagtttgccgctgcccaatgccatcctgga

SEQ ID NO: 272 - RNA, 26 nt, synthetic construct
  source 1..26: mol_type = other RNA
ccaatgccatcctggagttcctgtaa

SEQ ID NO: 273 - DNA, 25 bp, synthetic construct
  source 1..25: mol_type = other DNA
ccaatgccatcctggagttcctgta

SEQ ID NO: 274 - DNA, 25 bp, synthetic construct
  source 1..25: mol_type = other DNA
ctgacaacagtttgccgctgcccaa

SEQ ID NO: 275 - DNA, 22 bp, synthetic construct
  source 1..22: mol_type = other DNA
caatgccatcctggagttcctg

SEQ ID NO: 276 - DNA, 31 bp, synthetic construct
  source 1..31: mol_type = other DNA
gctgcccaatgccatcctggagttcctgtaa

SEQ ID NO: 277 - RNA, 26 nt, synthetic construct
  source 1..26: mol_type = other RNA
accagagtaacagtctgagtaggagc

SEQ ID NO: 278 - RNA, 23 nt, synthetic construct
  source 1..23: mol_type = other RNA
ctcataccttctgcttgatgatc

SEQ ID NO: 279 - RNA, 24 nt, synthetic construct
  source 1..24: mol_type = other RNA
ttctgtccaagcccggttgaaatc

SEQ ID NO: 280 - RNA, 30 nt, synthetic construct; identical to SEQ ID NO: 188
  source 1..30: mol_type = other RNA

SEQ ID NO: 281 - RNA, 25 nt, synthetic construct
  source 1..25: mol_type = other RNA
acatcaaggaagatggcatttctag

SEQ ID NO: 282 - RNA, 30 nt, synthetic construct; identical to SEQ ID NO: 181
  source 1..30: mol_type = other RNA

SEQ ID NO: 283 - RNA, 27 nt, synthetic construct
  source 1..27: mol_type = other RNA
atcattttttctcataccttctgctag

SEQ ID NO: 284 - RNA, 38 nt, synthetic construct
  source 1..38: mol_type = other RNA
atcattttttctcataccttctgctaggagctaaaaag

SEQ ID NO: 285 - RNA, 21 nt, synthetic construct
  source 1..21: mol_type = other RNA
cacccaccatcaccctctgtg

SEQ ID NO: 286 - RNA, 22 nt, synthetic construct
  source 1..22: mol_type = other RNA
atcatctcgttgatatcctcaa

SEQ ID NO: 287 - DNA, 30 bp, synthetic construct
  source 1..30: mol_type = other DNA
ctccaacatcaaggaagatggcatttctag

SEQ ID NO: 288 - DNA, 25 bp, synthetic construct
  source 1..25: mol_type = other DNA
ggccaaacctcggcttacctgaaat

SEQ ID NO: 289 - DNA, 23 bp, synthetic construct
  source 1..23: mol_type = other DNA
ccggttctgaaggtgttcttgta

SEQ ID NO: 290 - DNA, 24 bp, synthetic construct
  source 1..24: mol_type = other DNA
tccggttctgaaggtgttcttgta

SEQ ID NO: 291 - DNA, 25 bp, synthetic construct
  source 1..25: mol_type = other DNA
ctccggttctgaaggtgttcttgta

SEQ ID NO: 292 - DNA, 26 bp, synthetic construct
  source 1..26: mol_type = other DNA
cctccggttctgaaggtgttcttgta

SEQ ID NO: 293 - DNA, 27 bp, synthetic construct
  source 1..27: mol_type = other DNA
gcctccggttctgaaggtgttcttgta

SEQ ID NO: 294 - DNA, 28 bp, synthetic construct
  source 1..28: mol_type = other DNA
tgcctccggttctgaaggtgttcttgta

SEQ ID NO: 295 - DNA, 22 bp, synthetic construct
  source 1..22: mol_type = other DNA
ccggttctgaaggtgttcttgt

SEQ ID NO: 296 - DNA, 23 bp, synthetic construct
  source 1..23: mol_type = other DNA
tccggttctgaaggtgttcttgt

SEQ ID NO: 297 - DNA, 24 bp, synthetic construct
  source 1..24: mol_type = other DNA
ctccggttctgaaggtgttcttgt

SEQ ID NO: 298 - DNA, 25 bp, synthetic construct
  source 1..25: mol_type = other DNA
cctccggttctgaaggtgttcttgt

SEQ ID NO: 299 - DNA, 26 bp, synthetic construct
  source 1..26: mol_type = other DNA
gcctccggttctgaaggtgttcttgt

SEQ ID NO: 300 - DNA, 27 bp, synthetic construct
  source 1..27: mol_type = other DNA
tgcctccggttctgaaggtgttcttgt

SEQ ID NO: 301 - DNA, 21 bp, synthetic construct
  source 1..21: mol_type = other DNA
ccggttctgaaggtgttcttg

SEQ ID NO: 302 - DNA, 22 bp, synthetic construct
  source 1..22: mol_type = other DNA
tccggttctgaaggtgttcttg

SEQ ID NO: 303 - DNA, 23 bp, synthetic construct
  source 1..23: mol_type = other DNA
ctccggttctgaaggtgttcttg

SEQ ID NO: 304 - DNA, 24 bp, synthetic construct
  source 1..24: mol_type = other DNA
cctccggttctgaaggtgttcttg

SEQ ID NO: 305 - DNA, 25 bp, synthetic construct
  source 1..25: mol_type = other DNA
gcctccggttctgaaggtgttcttg

SEQ ID NO: 306 - DNA, 26 bp, synthetic construct
  source 1..26: mol_type = other DNA
tgcctccggttctgaaggtgttcttg

SEQ ID NO: 307 - DNA, 20 bp, synthetic construct
  source 1..20: mol_type = other DNA
ccggttctgaaggtgttctt

SEQ ID NO: 308 - DNA, 21 bp, synthetic construct
  source 1..21: mol_type = other DNA
tccggttctgaaggtgttctt

SEQ ID NO: 309 - DNA, 22 bp, synthetic construct
  source 1..22: mol_type = other DNA
ctccggttctgaaggtgttctt

SEQ ID NO: 310 - DNA, 23 bp, synthetic construct
  source 1..23: mol_type = other DNA
cctccggttctgaaggtgttctt

SEQ ID NO: 311 - DNA, 24 bp, synthetic construct
  source 1..24: mol_type = other DNA
gcctccggttctgaaggtgttctt

SEQ ID NO: 312 - DNA, 25 bp, synthetic construct
  source 1..25: mol_type = other DNA
tgcctccggttctgaaggtgttctt

SEQ ID NO: 313 - DNA, 19 bp, synthetic construct
  source 1..19: mol_type = other DNA
ccggttctgaaggtgttct

SEQ ID NO: 314 - DNA, 20 bp, synthetic construct
  source 1..20: mol_type = other DNA
tccggttctgaaggtgttct

SEQ ID NO: 315 - DNA, 21 bp, synthetic construct
  source 1..21: mol_type = other DNA
ctccggttctgaaggtgttct

SEQ ID NO: 316 - DNA, 22 bp, synthetic construct
  source 1..22: mol_type = other DNA
cctccggttctgaaggtgttct

SEQ ID NO: 317 - DNA, 23 bp, synthetic construct
  source 1..23: mol_type = other DNA
gcctccggttctgaaggtgttct

SEQ ID NO: 318 - DNA, 24 bp, synthetic construct
  source 1..24: mol_type = other DNA
tgcctccggttctgaaggtgttct

SEQ ID NO: 319 - DNA, 18 bp, synthetic construct
  source 1..18: mol_type = other DNA
ccggttctgaaggtgttc

SEQ ID NO: 320 - DNA, 19 bp, synthetic construct
  source 1..19: mol_type = other DNA
tccggttctgaaggtgttc

SEQ ID NO: 321 - DNA, 20 bp, synthetic construct
  source 1..20: mol_type = other DNA
ctccggttctgaaggtgttc

SEQ ID NO: 322 - DNA, 21 bp, synthetic construct
  source 1..21: mol_type = other DNA
cctccggttctgaaggtgttc

SEQ ID NO: 323 - DNA, 22 bp, synthetic construct
  source 1..22: mol_type = other DNA
gcctccggttctgaaggtgttc

SEQ ID NO: 324 - DNA, 23 bp, synthetic construct
  source 1..23: mol_type = other DNA
tgcctccggttctgaaggtgttc

SEQ ID NO: 325 - DNA, 25 bp, synthetic construct
  source 1..25: mol_type = other DNA
gttgcctccggttctgaaggtgttc

SEQ ID NO: 326 - DNA, 31 bp, synthetic construct
  source 1..31: mol_type = other DNA
cattcaactgttgcctccggttctgaaggtg

SEQ ID NO: 327 - DNA, 30 bp, synthetic construct
  source 1..30: mol_type = other DNA
ctgttgcctccggttctgaaggtgttcttg

SEQ ID NO: 328 - DNA, 30 bp, synthetic construct
  source 1..30: mol_type = other DNA
caactgttgcctccggttctgaaggtgttc

SEQ ID NO: 329 - DNA, 30 bp, synthetic construct
  source 1..30: mol_type = other DNA
ttgcctccggttctgaaggtgttcttgtac

SEQ ID NO: 330 - DNA, 25 bp, synthetic construct
  source 1..25: mol_type = other DNA
ctgaaggtgttcttgtacttcatcc

SEQ ID NO: 331 - DNA, 31 bp, synthetic construct; identical to SEQ ID NO: 326
  source 1..31: mol_type = other DNA

SEQ ID NO: 332 - RNA, 20 nt, synthetic construct
  source 1..20: mol_type = other RNA
tttgtgtctttctgagaaac

SEQ ID NO: 333 - RNA, 20 nt, synthetic construct
  source 1..20: mol_type = other RNA
aaagacttaccttaagatac

SEQ ID NO: 334 - RNA, 20 nt, synthetic construct
  source 1..20: mol_type = other RNA
atctgtcaaatcgcctgcag

SEQ ID NO: 335 - DNA, 30 bp, synthetic construct
  source 1..30: mol_type = other DNA
tgaaaacgccgccatttctcaacagatctg

SEQ ID NO: 336 - DNA, 30 bp, synthetic construct
  source 1..30: mol_type = other DNA
cataatgaaaacgccgccatttctcaacag

SEQ ID NO: 337 - DNA, 30 bp, synthetic construct
  source 1..30: mol_type = other DNA
tgttcagcttctgttagccactgattaaat

SEQ ID NO: 338 - DNA, 19 bp, synthetic construct
  source 1..19: mol_type = other DNA
cgccgccatttctcaacag

SEQ ID NO: 339 - DNA, 20 bp, synthetic construct
  source 1..20: mol_type = other DNA
atctgtcaaatcgcctgcag

SEQ ID NO: 340 - DNA, 15 bp, synthetic construct
  source 1..15: mol_type = other DNA
cagcagcagcagcag

SEQ ID NO: 341 - DNA, 18 bp, synthetic construct
  source 1..18: mol_type = other DNA
cagcagcagcagcagcag

SEQ ID NO: 342 - DNA, 21 bp, synthetic construct
  source 1..21: mol_type = other DNA
cagcagcagcagcagcagcag

SEQ ID NO: 343 - DNA, 24 bp, synthetic construct
  source 1..24: mol_type = other DNA
cagcagcagcagcagcagcagcag

SEQ ID NO: 344 - DNA, 15 bp, synthetic construct
  source 1..15: mol_type = other DNA
agcagcagcagcagc

SEQ ID NO: 345 - DNA, 18 bp, synthetic construct
  source 1..18: mol_type = other DNA
agcagcagcagcagcagc

SEQ ID NO: 346 - DNA, 21 bp, synthetic construct
  source 1..21: mol_type = other DNA
agcagcagcagcagcagcagc

SEQ ID NO: 347 - DNA, 24 bp, synthetic construct
  source 1..24: mol_type = other DNA
agcagcagcagcagcagcagcagc

SEQ ID NO: 348 - DNA, 15 bp, synthetic construct
  source 1..15: mol_type = other DNA
gcagcagcagcagca

SEQ ID NO: 349 - DNA, 18 bp, synthetic construct
  source 1..18: mol_type = other DNA
gcagcagcagcagcagca

SEQ ID NO: 350 - DNA, 21 bp, synthetic construct
  source 1..21: mol_type = other DNA
gcagcagcagcagcagcagca

SEQ ID NO: 351 - DNA, 24 bp, synthetic construct
  source 1..24: mol_type = other DNA
gcagcagcagcagcagcagcagca

SEQ ID NO: 352 - DNA, 16 bp, synthetic construct
  source 1..16: mol_type = other DNA
caggcaggcaggcagg

SEQ ID NO: 353 - DNA, 20 bp, synthetic construct
  source 1..20: mol_type = other DNA
caggcaggcaggcaggcagg

SEQ ID NO: 354 - DNA, 24 bp, synthetic construct
  source 1..24: mol_type = other DNA
caggcaggcaggcaggcaggcagg

SEQ ID NO: 355 - DNA, 28 bp, synthetic construct
  source 1..28: mol_type = other DNA
caggcaggcaggcaggcaggcaggcagg

SEQ ID NO: 356 - DNA, 32 bp, synthetic construct
  source 1..32: mol_type = other DNA
caggcaggcaggcaggcaggcaggcaggcagg

SEQ ID NO: 357 - DNA, 16 bp, synthetic construct
  source 1..16: mol_type = other DNA
aggcaggcaggcaggc

SEQ ID NO: 358 - DNA, 20 bp, synthetic construct
  source 1..20: mol_type = other DNA
aggcaggcaggcaggcaggc

SEQ ID NO: 359 - DNA, 24 bp, synthetic construct
  source 1..24: mol_type = other DNA
aggcaggcaggcaggcaggcaggc

SEQ ID NO: 360 - DNA, 28 bp, synthetic construct
  source 1..28: mol_type = other DNA
aggcaggcaggcaggcaggcaggcaggc

SEQ ID NO: 361 - DNA, 32 bp, synthetic construct
  source 1..32: mol_type = other DNA
aggcaggcaggcaggcaggcaggcaggcaggc

SEQ ID NO: 362 - DNA, 16 bp, synthetic construct
  source 1..16: mol_type = other DNA
ggcaggcaggcaggca

SEQ ID NO: 363 - DNA, 20 bp, synthetic construct
  source 1..20: mol_type = other DNA
ggcaggcaggcaggcaggca

SEQ ID NO: 364 - DNA, 24 bp, synthetic construct
  source 1..24: mol_type = other DNA
ggcaggcaggcaggcaggcaggca

SEQ ID NO: 365 - DNA, 28 bp, synthetic construct
  source 1..28: mol_type = other DNA
ggcaggcaggcaggcaggcaggcaggca

SEQ ID NO: 366 - DNA, 32 bp, synthetic construct
  source 1..32: mol_type = other DNA
ggcaggcaggcaggcaggcaggcaggcaggca

SEQ ID NO: 367 - DNA, 16 bp, synthetic construct
  source 1..16: mol_type = other DNA
gcaggcaggcaggcag

SEQ ID NO: 368 - DNA, 20 bp, synthetic construct
  source 1..20: mol_type = other DNA
gcaggcaggcaggcaggcag

SEQ ID NO: 369 - DNA, 24 bp, synthetic construct
  source 1..24: mol_type = other DNA
gcaggcaggcaggcaggcaggcag

SEQ ID NO: 370 - DNA, 28 bp, synthetic construct
  source 1..28: mol_type = other DNA
gcaggcaggcaggcaggcaggcaggcag

SEQ ID NO: 371 - DNA, 32 bp, synthetic construct
  source 1..32: mol_type = other DNA
gcaggcaggcaggcaggcaggcaggcaggcag

SEQ ID NO: 372 - DNA, 15 bp, synthetic construct
  source 1..15: mol_type = other DNA
ccagcattatgaaag

SEQ ID NO: 373 - DNA, 15 bp, synthetic construct
  source 1..15: mol_type = other DNA
ctagcaacatgaaag

SEQ ID NO: 374 - DNA, 15 bp, synthetic construct
  source 1..15: mol_type = other DNA
acaggccgatgaaag

SEQ ID NO: 375 - DNA, 15 bp, synthetic construct
  source 1..15: mol_type = other DNA
tgagaaccatgaaag

SEQ ID NO: 376 - DNA, 15 bp, synthetic construct
  source 1..15: mol_type = other DNA
cgagttagatgaaag

SEQ ID NO: 377 - DNA, 15 bp, synthetic construct
  source 1..15: mol_type = other DNA
ccaggggaatgaaag

SEQ ID NO: 378 - DNA, 15 bp, synthetic construct
  source 1..15: mol_type = other DNA
ccagaaggatgaaag

SEQ ID NO: 379 - DNA, 15 bp, synthetic construct
  source 1..15: mol_type = other DNA
cgagtctcatgaaag

SEQ ID NO: 380 - DNA, 15 bp, synthetic construct
  source 1..15: mol_type = other DNA
cgagcggtatgaaag

SEQ ID NO: 381 - DNA, 15 bp, synthetic construct
  source 1..15: mol_type = other DNA
ggagcggtatgaaag

SEQ ID NO: 382 - DNA, 15 bp, synthetic construct
  source 1..15: mol_type = other DNA
ccagaggtatgaaag

SEQ ID NO: 383 - DNA, 15 bp, synthetic construct
  source 1..15: mol_type = other DNA
ccagcggtatgaaag

SEQ ID NO: 384 - DNA, 15 bp, synthetic construct
  source 1..15: mol_type = other DNA
ccagcagtatgaaag

SEQ ID NO: 385 - DNA, 15 bp, synthetic construct
  source 1..15: mol_type = other DNA
taagccctatgaaag

SEQ ID NO: 386 - DNA, 15 bp, synthetic construct
  source 1..15: mol_type = other DNA
ctagttttatgaaag

SEQ ID NO: 387 - DNA, 15 bp, synthetic construct
  source 1..15: mol_type = other DNA
ccttaatttagaaag

SEQ ID NO: 388 - DNA, 15 bp, synthetic construct
  source 1..15: mol_type = other DNA
aaagcattatgaaag

SEQ ID NO: 389 - DNA, 15 bp, synthetic construct
  source 1..15: mol_type = other DNA
ttagcattatgaaag

SEQ ID NO: 390 - DNA, 15 bp, synthetic construct
  source 1..15: mol_type = other DNA
cgtgcattatgaaag

SEQ ID NO: 391 - DNA, 15 bp, synthetic construct
  source 1..15: mol_type = other DNA
ctgtcattatgaaag

SEQ ID NO: 392 - DNA, 15 bp, synthetic construct
  source 1..15: mol_type = other DNA
ctttcattatgaaag

SEQ ID NO: 393 - DNA, 15 bp, synthetic construct
  source 1..15: mol_type = other DNA
cattcattatgaaag

SEQ ID NO: 394 - DNA, 15 bp, synthetic construct
  source 1..15: mol_type = other DNA
ccagcattatgatta

SEQ ID NO: 395 - DNA, 15 bp, synthetic construct
  source 1..15: mol_type = other DNA
ccagcattatctaag

SEQ ID NO: 396 - DNA, 15 bp, synthetic construct
  source 1..15: mol_type = other DNA
ccagcattatcccag

SEQ ID NO: 397 - DNA, 15 bp, synthetic construct
  source 1..15: mol_type = other DNA
ccagcattattttag

SEQ ID NO: 398 - DNA, 20 bp, synthetic construct
  source 1..20: mol_type = other DNA
attcactttcataatgctgg

SEQ ID NO: 399 - RNA, 18 nt, synthetic construct
  source 1..18: mol_type = other RNA
tcactttcataatgctgg

SEQ ID NO: 400 - protein, 16 aa, synthetic construct
  SITE 1: note = acetyl group
  MOD_RES 2: note = bAla
  MOD_RES 5: note = bAla
  MOD_RES 12: note = bAla
  MOD_RES 14: note = bAla
  SITE 16: note = amidation of the alpha carboxyl group at E
RARRARFQILYARARE

SEQ ID NO: 401 - protein, 17 aa, synthetic construct
  SITE 1: note = acetyl group
  MOD_RES 2: note = bAla
  MOD_RES 5: note = bAla
  MOD_RES 13: note = bAla
  MOD_RES 15: note = bAla
  SITE 17: note = amidation of the alpha carboxyl group at E
RARRARFQILYRAHAHE

SEQ ID NO: 402 - protein, 14 aa, synthetic construct
  SITE 1: note = acetyl group
  MOD_RES 2: note = bAla
  MOD_RES 5: note = bAla
  MOD_RES 12: note = bAla
  SITE 14: note = amidation of the alpha carboxyl group at E
RARRARFQILYARE

SEQ ID NO: 403 - protein, 13 aa, synthetic construct
  SITE 1: note = acetyl group
  MOD_RES 2: note = bAla
  MOD_RES 5: note = bAla
  SITE 13: note = amidation of the alpha carboxyl group at E
RARRARRFQILYE

SEQ ID NO: 404 - protein, 13 aa, synthetic construct
  SITE 1: note = acetyl group
  MOD_RES 9: note = bAla
  MOD_RES 11: note = bAla
  SITE 13: note = amidation of the alpha carboxyl group at E
RRFQILYRAHAHE

SEQ ID NO: 405 - protein, 16 aa, synthetic construct
  SITE 1: note = acetyl group
  MOD_RES 2: note = bAla
  MOD_RES 5: note = bAla
  MOD_RES 12: note = bAla
  MOD_RES 14: note = bAla
  SITE 16: note = amidation of the alpha carboxyl group at E
  SITE 1: note = D-Arginine
  SITE 3..4: note = D-Arginine
  SITE 6: note = D-Arginine
  SITE 13: note = D-Arginine
  SITE 15: note = D-Arginine
  SITE 7: note = D-Phenylalanine
  SITE 8: note = D-Glutamine
  SITE 9: note = D-Isoleucine
  SITE 10: note = D-Leucine
  SITE 11: note = D-Tyrosine
  SITE 16: note = D-Glutamate
RARRARFQILYARARE

SEQ ID NO: 406 - DNA, 20 bp, synthetic construct
  source 1..20: mol_type = other DNA
caggccattcctctttcagg

SEQ ID NO: 407 - DNA, 21 bp, synthetic construct
  source 1..21: mol_type = other DNA
gaaactttcctcccagttggt

SEQ ID NO: 408 - DNA, 29 bp, synthetic construct
  source 1..29: mol_type = other DNA
  modified_base 1: mod_base = OTHER; note = 5'-hexachloro-fluorescein
  modified_base 29: mod_base = OTHER; note = 3'-IOWA BLACK FQ (registered trademark name)
  modified_base 8..9: mod_base = OTHER; note = ZEN internal quencher (trademark name)
tcaacttcagccatccatttctgtaaggt

SEQ ID NO: 409 - DNA, 30 bp, synthetic construct
  source 1..30: mol_type = other DNA
ctgaatatgaaataatggaggagagactcg

SEQ ID NO: 410 - DNA, 25 bp, synthetic construct
  source 1..25: mol_type = other DNA
cttcagccatccatttctgtaaggt

SEQ ID NO: 411 - DNA, 19 bp, synthetic construct
  source 1..19: mol_type = other DNA
  modified_base 1: mod_base = OTHER; note = 5'-carboxyfluorescein
  modified_base 19: mod_base = OTHER; note = 3'-IOWA BLACK FQ (registered trademark name)
  modified_base 9..10: mod_base = OTHER; note = ZEN internal quencher (trademark name)
atgtgattctgtaatttcc

SEQ ID NO: 412 - protein, 16 aa, synthetic construct
  MOD_RES 2: note = bAla
  MOD_RES 5: note = bAla
  MOD_RES 12: note = bAla
  MOD_RES 14: note = bAla
RARRARFQILYARARE

SEQ ID NO: 413 - protein, 17 aa, synthetic construct
  MOD_RES 2: note = bAla
  MOD_RES 5: note = bAla
  MOD_RES 13: note = bAla
  MOD_RES 15: note = bAla
RARRARFQILYRAHAHE

SEQ ID NO: 414 - protein, 17 aa, synthetic construct
  MOD_RES 2: note = bAla
  MOD_RES 5: note = bAla
  MOD_RES 13: note = bAla
  MOD_RES 15: note = bAla
  MOD_RES 17: note = bAla
RARRARFQILYRAHAHA

SEQ ID NO: 415 - protein, 14 aa, synthetic construct
  MOD_RES 2: note = bAla
  MOD_RES 5: note = bAla
  MOD_RES 12: note = bAla
RARRARFQILYARE

SEQ ID NO: 416 - protein, 13 aa, synthetic construct
  MOD_RES 2: note = bAla
  MOD_RES 5: note = bAla
RARRARRFQILYE

SEQ ID NO: 417 - protein, 13 aa, synthetic construct
  MOD_RES 9: note = bAla
  MOD_RES 11: note = bAla
RRFQILYRAHAHE

SEQ ID NO: 418 - protein, 16 aa, synthetic construct
  MOD_RES 2: note = bAla
  MOD_RES 5: note = bAla
  MOD_RES 12: note = bAla
  MOD_RES 14: note = bAla
  SITE 1: note = D-Arginine
  SITE 3: note = D-Arginine
  SITE 4: note = D-Arginine
  SITE 6: note = D-Arginine
  SITE 13: note = D-Arginine
  SITE 15: note = D-Arginine
  SITE 7: note = D-Phenylalanine
  SITE 8: note = D-Glutamine
  SITE 9: note = D-Isoleucine
  SITE 10: note = D-Leucine
  SITE 11: note = D-Tyrosine
  SITE 16: note = D-Glutamate
RARRARFQILYARARE

SEQ ID NO: 419 - protein, 4 aa, synthetic construct
  MOD_RES 2: note = bAla
RARR

SEQ ID NO: 420 - protein, 7 aa, synthetic construct
  MOD_RES 2: note = bAla
  MOD_RES 5: note = bAla
RARRARR

SEQ ID NO: 421 - protein, 5 aa, synthetic construct
  MOD_RES 3: note = bAla
RRARR

SEQ ID NO: 422 - protein, 9 aa, synthetic construct
  MOD_RES 3: note = bAla
  MOD_RES 6: note = bAla
  MOD_RES 9: note = bAla
HRAHRAHRA

SEQ ID NO: 423 - protein, 6 aa, synthetic construct
  MOD_RES 3: note = bAla
  MOD_RES 6: note = bAla
HRAHRA

SEQ ID NO: 424 - protein, 6 aa, synthetic construct
  MOD_RES 2: note = bAla
  MOD_RES 4: note = bAla
  MOD_RES 6: note = bAla
RARARA

SEQ ID NO: 425 - protein, 4 aa, synthetic construct
  MOD_RES 2: note = bAla
  MOD_RES 4: note = bAla
RARA

SEQ ID NO: 426 - protein, 6 aa, synthetic construct
HRHRHR

SEQ ID NO: 427 - protein, 4 aa, synthetic construct
HRHR

SEQ ID NO: 428 - protein, 6 aa, synthetic construct
RRRRRR

SEQ ID NO: 429 - protein, 6 aa, synthetic construct
  MOD_RES 2: note = bAla
  MOD_RES 5: note = bAla
RARRAR

SEQ ID NO: 430 - protein, 6 aa, synthetic construct
  MOD_RES 2: note = bAla
  MOD_RES 4: note = bAla
  MOD_RES 6: note = bAla
RAHAHA

SEQ ID NO: 431 - protein, 5 aa, synthetic construct
  MOD_RES 2: note = bAla
  MOD_RES 4: note = bAla
RAHAH

SEQ ID NO: 432 - protein, 5 aa, synthetic construct
  MOD_RES 1: note = bAla
  MOD_RES 3: note = bAla
  MOD_RES 5: note = bAla
AHAHA

SEQ ID NO: 433 - protein, 4 aa, synthetic construct
  MOD_RES 1: note = bAla
  MOD_RES 3: note = bAla
AHAH

SEQ ID NO: 434 - protein, 4 aa, synthetic construct
  MOD_RES 2: note = bAla
  MOD_RES 4: note = bAla
HAHA

SEQ ID NO: 435 - protein, 6 aa, synthetic construct
  MOD_RES 2: note = bAla
  MOD_RES 4: note = bAla
RAHAHE

SEQ ID NO: 436 - protein, 5 aa, synthetic construct
  MOD_RES 2: note = bAla
  MOD_RES 4: note = bAla
  MOD_RES 5: note = bAla
RAHAA

SEQ ID NO: 437 - protein, 4 aa, synthetic construct
  MOD_RES 2: note = bAla
  MOD_RES 4: note = bAla
RAHA

SEQ ID NO: 438 - protein, 6 aa, synthetic construct
  MOD_RES 3: note = bAla
  MOD_RES 6: note = bAla
HRARHA

SEQ ID NO: 439 - protein, 8 aa, synthetic construct
  MOD_RES 3: note = bAla
  MOD_RES 6: note = bAla
HRAHRAHR

SEQ ID NO: 440 - protein, 5 aa, synthetic construct
  MOD_RES 3: note = bAla
HRAHR

SEQ ID NO: 441 - protein, 5 aa, synthetic construct
  MOD_RES 2: note = bAla
  MOD_RES 4: note = bAla
RARAR

SEQ ID NO: 442 - protein, 7 aa, synthetic construct
  MOD_RES 7: note = bAla
HRHRHRA

SEQ ID NO: 443 - protein, 5 aa, synthetic construct
  MOD_RES 5: note = bAla
HRHRA

SEQ ID NO: 444 - protein, 5 aa, synthetic construct
  MOD_RES 3: note = bAla
HRARH

SEQ ID NO: 445 - protein, 7 aa, synthetic construct
  MOD_RES 7: note = bAla
RRRRRRA

SEQ ID NO: 446 - protein, 5 aa, synthetic construct
FQILY

SEQ ID NO: 447 - protein, 14 aa, synthetic construct
  MOD_RES 2: note = bAla
  MOD_RES 11: note = bAla
  MOD_RES 13: note = bAla
RARRFQILYRAHAH

SEQ ID NO: 448 - protein, 13 aa, synthetic construct
  MOD_RES 2: note = bAla
  MOD_RES 10: note = bAla
  MOD_RES 12: note = bAla
RARFQILYRAHAH

SEQ ID NO: 449 - protein, 12 aa, synthetic construct
  MOD_RES 2: note = bAla
  MOD_RES 11: note = bAla
  MOD_RES 9: note = bAla
RAFQILYRAHAH

SEQ ID NO: 450 - protein, 11 aa, synthetic construct
  MOD_RES 8: note = bAla
  MOD_RES 10: note = bAla
RFQILYRAHAH

SEQ ID NO: 451 - protein, 10 aa, synthetic construct
  MOD_RES 7: note = bAla
  MOD_RES 9: note = bAla
FQILYRAHAH

SEQ ID NO: 452 - protein, 17 aa, synthetic construct
  MOD_RES 2: note = bAla
  MOD_RES 5: note = bAla
  MOD_RES 13: note = bAla
  MOD_RES 15: note = bAla
  MOD_RES 17: note = bAla
RARRARRFQILYAHAHA

SEQ ID NO: 453 - protein, 15 aa, synthetic construct
  MOD_RES 2: note = bAla
  MOD_RES 5: note = bAla
  MOD_RES 14: note = bAla
RARRARRFQILYHAH

SEQ ID NO: 454 - protein, 14 aa, synthetic construct
  MOD_RES 2: note = bAla
  MOD_RES 5: note = bAla
  MOD_RES 13: note = bAla
RARRARRFQILYAH

SEQ ID NO: 455 - protein, 13 aa, synthetic construct
  MOD_RES 2: note = bAla
  MOD_RES 5: note = bAla
RARRARRFQILYH

SEQ ID NO: 456 - protein, 12 aa, synthetic construct
  MOD_RES 2: note = bAla
  MOD_RES 5: note = bAla
RARRARRFQILY

SEQ ID NO: 457 - protein, 16 aa, synthetic construct
  MOD_RES 1: note = bAla
  MOD_RES 4: note = bAla
  MOD_RES 13: note = bAla
  MOD_RES 15: note = bAla
ARRARRFQILYRAHAH

SEQ ID NO: 458 - protein, 15 aa, synthetic construct
  MOD_RES 3: note = bAla
  MOD_RES 12: note = bAla
  MOD_RES 14: note = bAla
RRARRFQILYRAHAH

SEQ ID NO: 459 - protein, 13 aa, synthetic construct
  MOD_RES 1: note = bAla
  MOD_RES 10: note = bAla
  MOD_RES 12: note = bAla
ARRFQILYRAHAH

SEQ ID NO: 460 - protein, 12 aa, synthetic construct
  MOD_RES 9: note = bAla
  MOD_RES 11: note = bAla
RRFQILYRAHAH

SEQ ID NO: 461 - protein, 16 aa, synthetic construct
  MOD_RES 2: note = bAla
  MOD_RES 5: note = bAla
  MOD_RES 14: note = bAla
  MOD_RES 16: note = bAla
RARRARRFQILYRAHA

SEQ ID NO: 462 - protein, 15 aa, synthetic construct
  MOD_RES 2: note = bAla
  MOD_RES 5: note = bAla
  MOD_RES 14: note = bAla
RARRARRFQILYRAH

SEQ ID NO: 463 - protein, 14 aa, synthetic construct
  MOD_RES 2: note = bAla
  MOD_RES 5: note = bAla
  MOD_RES 14: note = bAla
RARRARRFQILYRA

SEQ ID NO: 464 - protein, 13 aa, synthetic construct
  MOD_RES 2: note = bAla
  MOD_RES 5: note = bAla
RARRARRFQILYR

SEQ ID NO: 465 - protein, 19 aa, synthetic construct
  MOD_RES 3: note = bAla
  MOD_RES 6: note = bAla
  MOD_RES 9: note = bAla
  MOD_RES 17: note = bAla
HRAHRAHRAFQILYHRARH

SEQ ID NO: 466 - protein, 22 aa, synthetic construct
  MOD_RES 3: note = bAla
  MOD_RES 6: note = bAla
  MOD_RES 9: note = bAla
  MOD_RES 17: note = bAla
  MOD_RES 20: note = bAla
HRAHRAHRAFQILYHRAHRAHR

SEQ ID NO: 467 - protein, 16 aa, synthetic construct
  MOD_RES 3: note = bAla
  MOD_RES 6: note = bAla
  MOD_RES 14: note = bAla
HRAHRAFQILYHRAHR

SEQ ID NO: 468 - protein, 10 aa, synthetic construct
  MOD_RES 3: note = bAla
HRAFQILYHR

SEQ ID NO: 469 - protein, 16 aa, synthetic construct
  MOD_RES 2: note = bAla
  MOD_RES 4: note = bAla
  MOD_RES 6: note = bAla
  MOD_RES 13: note = bAla
  MOD_RES 15: note = bAla
RARARAFQILYRARAR

SEQ ID NO: 470 - protein, 12 aa, synthetic construct
  MOD_RES 2: note = bAla
  MOD_RES 4: note = bAla
  MOD_RES 11: note = bAla
RARAFQILYRAR

SEQ ID NO: 471 - protein, 8 aa, synthetic construct
  MOD_RES 2: note = bAla
RAFQILYR

SEQ ID NO: 472 - protein, 17 aa, synthetic construct
HRHRHRFQILYHRHRHR

SEQ ID NO: 473 - protein, 13 aa, synthetic construct
HRHRFQILYHRHR

SEQ ID NO: 474 - protein, 9 aa, synthetic construct
HRFQILYHR

SEQ ID NO: 475 - protein, 11 aa, synthetic construct
RRRRRRFQILY

SEQ ID NO: 476 - protein, 11 aa, synthetic construct
FQILYRRRRRR

SEQ ID NO: 477 - protein, 17 aa, synthetic construct
RRRRRRFQILYRRRRRR

SEQ ID NO: 478 - protein, 13 aa, synthetic construct
  MOD_RES 2: note = bAla
  MOD_RES 5: note = bAla
  MOD_RES 12: note = bAla
RARRARFQILYAR

SEQ ID NO: 479 - protein, 11 aa, synthetic construct
  MOD_RES 2: note = bAla
  MOD_RES 5: note = bAla
RARRARFQILY

SEQ ID NO: 480 - skipped

SEQ ID NO: 481 - protein, 15 aa, synthetic construct
  MOD_RES 2: note = bAla
  MOD_RES 11: note = bAla
  MOD_RES 13: note = bAla
  MOD_RES 15: note = bAla
RARRFQILYRAHAHA

SEQ ID NO: 482 - protein, 14 aa, synthetic construct
  MOD_RES 2: note = bAla
  MOD_RES 10: note = bAla
  MOD_RES 12: note = bAla
  MOD_RES 14: note = bAla
RARFQILYRAHAHA

SEQ ID NO: 483 - protein, 13 aa, synthetic construct
  MOD_RES 2: note = bAla
  MOD_RES 9: note = bAla
  MOD_RES 11: note = bAla
  MOD_RES 13: note = bAla
RAFQILYRAHAHA

SEQ ID NO: 484 - protein, 12 aa, synthetic construct
  MOD_RES 8: note = bAla
  MOD_RES 10: note = bAla
  MOD_RES 12: note = bAla
RFQILYRAHAHA

SEQ ID NO: 485 - protein, 11 aa, synthetic construct
  MOD_RES 7: note = bAla
  MOD_RES 9: note = bAla
  MOD_RES 11: note = bAla
FQILYRAHAHA

SEQ ID NO: 486 - protein, 16 aa, synthetic construct
  MOD_RES 2: note = bAla
  MOD_RES 5: note = bAla
  MOD_RES 14: note = bAla
  MOD_RES 16: note = bAla
RARRARRFQILYHAHA

SEQ ID NO: 487 - protein, 15 aa, synthetic construct
  MOD_RES 2: note = bAla
  MOD_RES 5: note = bAla
  MOD_RES 13: note = bAla
  MOD_RES 15: note = bAla
RARRARRFQILYAHA

SEQ ID NO: 488 - protein, 14 aa, synthetic construct
  MOD_RES 2: note = bAla
  MOD_RES 5: note = bAla
  MOD_RES 14: note = bAla
RARRARRFQILYHA

SEQ ID NO: 489 - protein, 13 aa, synthetic construct
  MOD_RES 2: note = bAla
  MOD_RES 5: note = bAla
  MOD_RES 13: note = bAla
RARRARRFQILYA

SEQ ID NO: 490 - protein, 17 aa, synthetic construct
  MOD_RES 1: note = bAla
  MOD_RES 4: note = bAla
  MOD_RES 13: note = bAla
  MOD_RES 15: note = bAla
  MOD_RES 17: note = bAla
ARRARRFQILYRAHAHA

SEQ ID NO: 491 - protein, 16 aa, synthetic construct
  MOD_RES 3: note = bAla
  MOD_RES 12: note = bAla
  MOD_RES 14: note = bAla
  MOD_RES 16: note = bAla
RRARRFQILYRAHAHA

SEQ ID NO: 492 - protein, 14 aa, synthetic construct
  MOD_RES 1: note = bAla
  MOD_RES 10: note = bAla
  MOD_RES 12: note = bAla
  MOD_RES 14: note = bAla
ARRFQILYRAHAHA

SEQ ID NO: 493 - protein, 13 aa, synthetic construct
  MOD_RES 9: note = bAla
  MOD_RES 11: note = bAla
  MOD_RES 13: note = bAla
RRFQILYRAHAHA

SEQ ID NO: 494 - protein, 17 aa, synthetic construct
  MOD_RES 2: note = bAla
  MOD_RES 5: note = bAla
  MOD_RES 14: note = bAla
  MOD_RES 16: note = bAla
  MOD_RES 17: note = bAla
RARRARRFQILYRAHAA

SEQ ID NO: 495 - protein, 15 aa, synthetic construct
  MOD_RES 2: note = bAla
  MOD_RES 5: note = bAla
  MOD_RES 14: note = bAla
  MOD_RES 15: note = bAla
RARRARRFQILYRAA

SEQ ID NO: 496 - protein, 20 aa, synthetic construct
  MOD_RES 3: note = bAla
  MOD_RES 6: note = bAla
  MOD_RES 9: note = bAla
  MOD_RES 17: note = bAla
  MOD_RES 20: note = bAla
HRAHRAHRAFQILYHRARHA

SEQ ID NO: 497 - protein, 23 aa, synthetic construct
  MOD_RES 3: note = bAla
  MOD_RES 6: note = bAla
  MOD_RES 9: note = bAla
  MOD_RES 17: note = bAla
  MOD_RES 20: note = bAla
  MOD_RES 23: note = bAla
HRAHRAHRAFQILYHRAHRAHRA

SEQ ID NO: 498 - protein, 17 aa, synthetic construct
  MOD_RES 3: note = bAla
  MOD_RES 6: note = bAla
  MOD_RES 14: note = bAla
  MOD_RES 17: note = bAla
HRAHRAFQILYHRAHRA

SEQ ID NO: 499 - protein, 11 aa, synthetic construct
  MOD_RES 3: note = bAla
  MOD_RES 11: note = bAla
HRAFQILYHRA

SEQ ID NO: 500 - protein, 17 aa, synthetic construct
  MOD_RES 2: note = bAla
  MOD_RES 4: note = bAla
  MOD_RES 6: note = bAla
  MOD_RES 13: note = bAla
  MOD_RES 15: note = bAla
  MOD_RES 17: note = bAla
RARARAFQILYRARARA

SEQ ID NO: 501 - protein, 13 aa, synthetic construct
  MOD_RES 2: note = bAla
  MOD_RES 4: note = bAla
  MOD_RES 11: note = bAla
  MOD_RES 13: note = bAla
RARAFQILYRARA

SEQ ID NO: 502 - protein, 9 aa, synthetic construct
  MOD_RES 2: note = bAla
  MOD_RES 9: note = bAla
RAFQILYRA

SEQ ID NO: 503 - protein, 18 aa, synthetic construct
  MOD_RES 18: note = bAla
HRHRHRFQILYHRHRHRA

SEQ ID NO: 504 - protein, 14 aa, synthetic construct
  MOD_RES 14: note = bAla
HRHRFQILYHRHRA

SEQ ID NO: 505 - protein, 10 aa, synthetic construct
  MOD_RES 10: note = bAla
HRFQILYHRA

SEQ ID NO: 506 - protein, 12 aa, synthetic construct
  MOD_RES 12: note = bAla
RRRRRRFQILYA

SEQ ID NO: 507 - protein, 12 aa, synthetic construct
  MOD_RES 12: note = bAla
FQILYRRRRRRA

SEQ ID NO: 508 - protein, 18 aa, synthetic construct
  MOD_RES 18: note = bAla
RRRRRRFQILYRRRRRRA

SEQ ID NO: 509 - protein, 12 aa, synthetic construct
  MOD_RES 2: note = bAla
  MOD_RES 5: note = bAla
RARRARFQILYE

SEQ ID NO: 510 - protein, 15 aa, synthetic construct
  MOD_RES 2: note = bAla
  MOD_RES 5: note = bAla
  MOD_RES 12: note = bAla
  MOD_RES 14: note = bAla
  SITE 1: note = D-Arginine
  SITE 3: note = D-Arginine
  SITE 4: note = D-Arginine
  SITE 6: note = D-Arginine
  SITE 13: note = D-Arginine
  SITE 15: note = D-Arginine
  SITE 7: note = D-Phenylalanine
  SITE 8: note = D-Glutamine
  SITE 9: note = D-Isoleucine
  SITE 10: note = D-Leucine
  SITE 11: note = D-Tyrosine
RARRARFQILYARAR

SEQ ID NO: 511 - protein, 17 aa, synthetic construct
  SITE 1: note = acetyl group
  MOD_RES 2: note = bAla
  MOD_RES 5: note = bAla
  MOD_RES 13: note = bAla
  MOD_RES 15: note = bAla
  MOD_RES 17: note = bAla
  SITE 17: note = hydroxylation of the alpha carboxyl group at A
RARRARFQILYRAHAHA

SEQ ID NO: 512 - protein, 12 aa, synthetic construct
  SITE 1: note = acetyl group
  MOD_RES 2: note = bAla
  MOD_RES 5: note = bAla
  SITE 12: note = amidation of the alpha carboxyl group at E
RARRARFQILYE

SEQ ID NO: 513 - protein, 5 aa, synthetic construct
YQFLI

SEQ ID NO: 514 - protein, 5 aa, synthetic construct
IQFLI

SEQ ID NO: 515 - protein, 5 aa, synthetic construct
YRFLI

SEQ ID NO: 516 - protein, 5 aa, synthetic construct
ILFRY

(19) United States
(12) Patent Application Publication
(10) Pub. No.: US 2026/0234616 A1
(43) Pub. Date: Aug. 13, 2026

(54) CELL-PENETRATING PEPTIDES, CONJUGATES THEREOF, AND METHODS OF THEIR USE

(71) Applicant: PepGen Inc., Boston, MA (US)

(72) Inventors: Ashling HOLLAND, Dublin (IE); Smita GUNNOO, Oxford (GB); Robert HAMMER, Maynard, MA (US); Niels SVENSTRUP, Brookline, MA (US); Caroline GODFREY, Oxford (GB); Sonia BRACEGIRDLE, Christchurch (NZ)

(21) Appl. No.: 19/131,778

(22) PCT Filed: Nov. 21, 2023
(86) PCT No.: PCT/US2023/080778

§ 371 Date: May 21, 2025

Related U.S. Application Data
(60) Provisional application No. 63/426,854, filed on Nov. 21, 2022.

(51) Int. Cl. C12N 15/113; A61K 31/712; A61K 31/7125; A61K 47/64; A61P 21/00

(52) U.S. Cl. CPC C12N 15/113; A61K 31/712; A61K 31/7125; A61K 47/6455; A61P 21/00; C12N 2310/11; C12N 2310/315; C12N 2310/3181; C12N 2310/321; C12N 2310/3233; C12N 2310/3513; C12N 2320/33

(57) ABSTRACT

Disclosed are peptides and conjugates of the peptide covalently bonded or linked via a linker to a therapeutic or diagnostic molecule, the peptide including at least one cationic domain and at least one hydrophobic domain.

### FIELD OF THE INVENTION

The invention relates to cell-penetrating peptides, conjugates thereof, compositions containing them, and methods of their use.

### BACKGROUND

Delivery of exogenous biomolecules, such as therapeutic and diagnostic molecules, through the cell membrane is essential for these biomolecules to have their intended effects within cells. The cell membrane functions as a biochemical barrier, and thus having exogenous biomolecules reach their intended targets can be challenging. Certain approaches for the delivery of biomolecules into cells may result in toxicity, poor specificity, poor stability, immunogenicity, low efficacy, and low delivery efficiency. Accordingly, there is a need for the development of new approaches for safe and efficient delivery of biomolecules into target cells.

Nucleic acid drugs are genomic medicines with the potential to transform human healthcare. Research has indicated that such therapeutics could have applications across a broad range of disease areas including genetic disorders, such as certain neuromuscular and neurologic diseases. Oligonucleotide therapeutics are a nucleic acid-based genetic medicine modality that are designed to target the root cause of many diseases through the modulation of RNA expression and processing. The mechanisms of action of these medicines include interference with gene expression; degradation of toxic RNA species; alteration of gene translation; interference with interactions between RNA and other nucleic acids or proteins; endogenous human adenosine deaminase acting on RNA, or ADAR; site-directed RNA editing; and modulation of the splicing of genes, and each of these approaches can lead to profound biological effects. The application of antisense oligonucleotide-based methods to modulate pre-mRNA splicing in the neuromuscular disease Duchenne muscular dystrophy (DMD) has placed this monogenic disorder at the forefront of advances in precision medicine. Additional examples of genetic diseases that may be amenable to treatment with oligonucleotide therapeutic approaches include facioscapulohumeral muscular dystrophy (FSHD), myotonic dystrophy type 1 (DM1), myotonic dystrophy type 2 (DM2), Charcot-Marie-Tooth type 1a (CMT1a), Charcot-Marie-Tooth type 2a (CMT2a), Fuch's corneal dystrophy (FCD), Friedreich's ataxia (FA), and spinal muscular atrophy (SMA).

However, therapeutic development of these promising antisense oligonucleotide therapeutics has been hampered by insufficient cell-penetrance and poor distribution characteristics—a challenge that is further emphasized by the large volume and dispersed nature of the muscle tissue substrate in DMD and other neuromuscular diseases, and the difficulties in accessing the afflicted central and peripheral nervous system in neurologic diseases.

DMD affects approximately one in 3,500 newborn boys. This severe, X-linked recessive disease results from mutations in the DMD gene that encodes dystrophin protein. The disorder is characterized by progressive muscle degeneration and wasting, along with the emergence of respiratory failure and cardiac complications, ultimately leading to premature death. Most mutations underlying DMD are genomic out-of-frame deletions that induce a premature truncation in the open reading frame that results in the absence of the dystrophin protein.

Exon skipping therapy utilizes splice switching antisense oligonucleotides (SSOs) to target specific regions of the DMD transcript, inducing the exclusion of individual exons, leading to the restoration of aberrant reading frames and resulting in the production of an internally deleted, yet partially functional, dystrophin protein. Despite the undoubted potential of antisense oligonucleotide exon skipping therapy for DMD, the successful application of this approach is currently limited by the relatively poor uptake of oligonucleotides by skeletal muscle, as well as the inadequate targeting of single stranded oligonucleotides to other affected tissues such as the heart. In September 2016 the Food and Drug Administration (FDA) granted accelerated approval for eteplirsen, a modulator of exon 51 splicing. Although this heralded the first US FDA-approved oligonucleotide that modulates splicing, and the first approved drug for DMD, the levels of dystrophin restoration in patients were minimal, with approximately 1% of normal being reported by the sponsor. Comparisons with the allelic disorder Becker muscular dystrophy (BMD) and experiments in the canonical mdx mouse have indicated that homogenous sarcolemmal dystrophin expression of at least 10-15% of wild type is needed to protect muscle against exercise induced damage.

Therefore, there is a need for new approaches for delivering therapeutic and diagnostic molecules into cells. Such approaches can be used, for example, for the delivery of antisense oligonucleotide-based therapeutics for devastating genetic diseases including neuromuscular diseases, such as DMD and others, and neurologic diseases.

### SUMMARY OF THE INVENTION

In general, the invention provides a conjugate of a peptide and a therapeutic or diagnostic molecule covalently bonded or covalently linked via a linker. In some embodiments, the therapeutic or diagnostic molecule can be an oligonucleotide that may be complementary to a target sequence within a transcript, the altered processing or recognition or steric blocking of which leads to beneficial therapeutic effects. In some embodiments, the target sequence is associated with a genetic disease, such as a neuromuscular or neurologic disease (e.g., DMD, BMD, DM1, DM2, CMT1a, CMT2a, FCD, FA, or SMA). For example, the oligonucleotide may be complementary to a target sequence within or proximal to any one of exons 8-55 (e.g., exon 8, exon 23, exon 44, exon 45, exon 50, exon 51, exon 52, exon 53, or exon 55) of a dystrophin, i.e., DMD, transcript (e.g., a human dystrophin transcript). In other examples, the oligonucleotide may be complementary to a r(CUG)^{exp} (e.g., the oligonucleotide may have at least 9 contiguous nucleobases complementary to a CUG repeat sequence; also see below) in the 3′-untranslated region of the DMPK transcript. In other examples, the oligonucleotide may be complementary to a r(CCUG)^{exp} sequence in the CNBP transcript. Alternatively, the oligonucleotide may be complementary to a target sequence within or proximal to intron 7 of a human survival of motor neuron 2 (SMN2) transcript, or to target sequences located within a DUX4 transcript, a PMP22 transcript, a MFN2 transcript, a TCF4 transcript or a FXN transcript.

In one aspect, the conjugate is of a peptide and a therapeutic or diagnostic molecule (e.g., an oligonucleotide) covalently bonded (i.e., directly) or linked via a non-cationic linker (e.g., via a linker including a total of one amino acid or via an aliphatic dicarboxylic acid linker) to the peptide, the peptide including a total of one hydrophobic domain and a total of one cationic domain, the cationic domain including at least 1 cationic amino acid residue, and the hydrophobic domain including at least 3 amino acid residues, provided that the peptide includes a total of 5 to 40 (e.g., 6 to 40 or 7 to 40) amino acid residues and does not include any artificial amino acid residues. In some embodiments, the amino acid residues for each cationic domain are selected from the group consisting of arginine, histidine, and beta-alanine.

In another aspect, the conjugate is of a peptide and a therapeutic or diagnostic molecule (e.g., an oligonucleotide) covalently bonded (i.e., directly) or linked via a non-cationic linker (e.g., via a linker including a total of one amino acid or via an aliphatic dicarboxylic acid linker) to the peptide, the peptide including a total of one hydrophobic domain and a total of one or two cationic domains flanking the hydrophobic domain, each cationic domain including at least one cationic amino acid residue, at least one of the cationic domains including a total of one cationic amino acid residue, and the hydrophobic domain including at least 3 amino acid residues, provided that the peptide includes a total of 5 to 40 (e.g., 6 to 40 or 7 to 40) amino acid residues and does not include any artificial amino acid residues. In some embodiments, the amino acid residues for each cationic domain are selected from the group consisting of arginine, histidine, and beta-alanine. In some embodiments, at least one cationic domain includes three or fewer amino acid residues.

In yet another aspect, the conjugate is of a peptide and a therapeutic or diagnostic molecule (e.g., an oligonucleotide) covalently bonded (i.e., directly) or linked via a non-cationic linker (e.g., via a linker including a total of one amino acid or via an aliphatic dicarboxylic acid linker) to the peptide, the peptide including a total of one hydrophobic domain and a total of one or two cationic domains flanking the hydrophobic domain, each cationic domain including at least 1 cationic amino acid residue, all cationic domains collectively including a total of five or fewer cationic amino acid residue, and the hydrophobic domain including at least 3 amino acid residues, provided that the peptide includes a total of 5 to 40 (e.g., 6 to 40 or 7 to 40) amino acid residues and does not include any artificial amino acid residues. In some embodiments, the amino acid residues for each cationic domain are selected from the group consisting of arginine, histidine, and beta-alanine. In some embodiments, at least one cationic domain includes three or fewer amino acid residues.

In still another aspect, the conjugate is of a peptide and a therapeutic or diagnostic molecule (e.g., an oligonucleotide) covalently bonded (i.e., directly) or linked via a non-cationic linker (e.g., via a linker including a total of one amino acid or via an aliphatic dicarboxylic acid linker) to the peptide, the peptide including a total of one hydrophobic domain and a total of one or two cationic domains flanking the hydrophobic domain, each cationic domain including at least 1 cationic amino acid residue, at least one third of amino acid residues in the N-terminal cationic domain are histidines, and the hydrophobic domain including at least 3 amino acid residues, provided that the peptide includes a total of 5 to 40 (e.g., 6 to 40 or 7 to 40) amino acid residues and does not include any artificial amino acid residues. In some embodiments, the amino acid residues for each cationic domain are selected from the group consisting of arginine, histidine, and beta-alanine. In some embodiments, at least one cationic domain includes three or fewer amino acid residues.

In some embodiments, each cationic domain includes at least 40%, at least 45%, or at least 50% cationic amino acids. In some embodiments, each cationic domain includes a majority of cationic amino acids, preferably at least at least 55%, at least 60%, at least 65% at least 70%, at least 75%, at least 80%, at least 85%, at least 90%, at least 95% cationic amino acids. In some embodiments, each cationic domain includes arginine, histidine, beta-alanine, hydroxyproline and/or serine residues, preferably wherein each cationic domain consists of arginine, histidine, beta-alanine, hydroxyproline and/or serine residues, provided at least one arginine or histidine is present. In some embodiments, each cationic domain is arginine rich and/or histidine rich, preferably each cationic domain includes at least 40%, at least 45%, at least 50%, at least 55%, at least 60%, at least 60%, at least 65%, or least 70% arginine and/or histidine residues. In some embodiments, at least one cationic domain (e.g., all cationic domains) is selected from the group consisting of RBRR (SEQ ID NO: 419), RBR, RB, R, RBRRBRR (SEQ ID NO: 420), RRBRR (SEQ ID NO: 421), BRR, RR, HRBHRBHRB (SEQ ID NO: 422), HRBHRB (SEQ ID NO: 423), HRB, RBRBRB (SEQ ID NO: 424), RBRB (SEQ ID NO: 425), RB, HRHRHR (SEQ ID NO: 426), HRHR (SEQ ID NO: 427), HR, RRRRRR (SEQ ID NO: 428), RBRRBR (SEQ ID NO: 429), RBHBHB (SEQ ID NO: 430), RBHBH (SEQ ID NO: 431), BHBHB (SEQ ID NO: 432), BHBH (SEQ ID NO: 433), HBHB (SEQ ID NO: 434), HBH, BHB, BH, HB, H, RBHBHE (SEQ ID NO: 435), RBHBB (SEQ ID NO: 436), RBHB (SEQ ID NO: 437), RBB, HRBRHB (SEQ ID NO: 438), HRBHRBHR (SEQ ID NO: 439), HRBHR (SEQ ID NO: 440), HRB, RBRBR (SEQ ID NO: 441), HRHRHRB (SEQ ID NO: 442), HRHRB (SEQ ID NO: 443), HRBRH (SEQ ID NO: 444), HRB, RRRRRRB (SEQ ID NO: 445), BRE, and BR, e.g., RBRR (SEQ ID NO: 419), RBR, RB, R, RBRRBRR (SEQ ID NO: 420), RRBRR (SEQ ID NO: 421), BRR, RR, HRBHRBHRB (SEQ ID NO: 422), HRBHRB (SEQ ID NO: 423), HRB, RBRBRB (SEQ ID NO: 424), RBRB (SEQ ID NO: 425), RB, HRHRHR (SEQ ID NO: 426), HRHR (SEQ ID NO: 427), HR, RRRRRR (SEQ ID NO: 428), RBRRBR (SEQ ID NO: 429), RBHBH (SEQ ID NO: 431), BHBH (SEQ ID NO: 433), HBH, BH, H, RBHB (SEQ ID NO: 437), HRBRH (SEQ ID NO: 444), HRBHRBHR (SEQ ID NO: 439), HRBHR (SEQ ID NO: 440), RBRBR (SEQ ID NO: 441), and BR. In some embodiments, at least one cationic domain (e.g., all cationic domains) is selected from the group consisting of RBR, RBRB (SEQ ID NO: 425), RB, R, RBRRBRR (SEQ ID NO: 420), BRR, BR, RR, HRBHRBHRB (SEQ ID NO: 422), HRBHRB (SEQ ID NO: 423), HRBHRBHR (SEQ ID NO: 439), HRBHR (SEQ ID NO: 440), HRB, HRHRHR (SEQ ID NO: 426), HR, RRRRRR (SEQ ID NO: 428), RRRRRRB (SEQ ID NO: 445), RBHBH (SEQ ID NO: 431), BH, BHB, H, HB, RBH, and RBHB (SEQ ID NO: 437), e.g., RBR, R, RBRRBRR (SEQ ID NO: 420), BRR, BR, RR, HRBHRBHR (SEQ ID NO: 439), HRBHR (SEQ ID NO: 440), HRHRHR (SEQ ID NO: 426), HR, RRRRRR (SEQ ID NO: 428), RBHBH (SEQ ID NO: 431), BH, H, and RBH.

In some embodiments, each hydrophobic domain has a length of between 3-6 amino acids, preferably each hydrophobic domain has a length of 5 amino acids. In some embodiments, each hydrophobic domain includes a majority of hydrophobic amino acid residues, preferably each hydrophobic domain includes at least 70%, at least 75%, at least 80%, at least 85%, at least 90%, at least 95%, or 100% hydrophobic amino acids. In some embodiments, each hydrophobic domain includes phenylalanine, leucine, isoleucine, tyrosine, tryptophan, proline, and glutamine residues; preferably wherein each hydrophobic domain consists of phenylalanine, leucine, isoleucine, tyrosine, tryptophan, proline, and/or glutamine residues, provided at least one of phenylalanine, leucine, isoleucine, tyrosine, and tryptophan is present. In some embodiments, the peptide includes one hydrophobic domain. In some embodiments, the hydrophobic domain is FQILY (SEQ ID NO: 446).

In another aspect, the conjugate is of a peptide and a therapeutic or diagnostic molecule covalently bonded (i.e., directly) or linked via a non-cationic linker (e.g., via a linker including a total of one amino acid or via an aliphatic dicarboxylic acid linker) to the peptide, the peptide being selected from the group consisting of: RBRRFQILYRBHBH (SEQ ID NO: 447), RBRFQILYRBHBH (SEQ ID NO: 448), RBFQILYRBHBH (SEQ ID NO: 449), RFQILYRBHBH (SEQ ID NO: 450), FQILYRBHBH (SEQ ID NO: 451), RBRRBRRFQILYBHBHB (SEQ ID NO: 452), RBRRBRRFQILYHBH (SEQ ID NO: 453), RBRRBRRFQILYBH (SEQ ID NO: 454), RBRRBRRFQILYH (SEQ ID NO: 455), RBRRBRRFQILY (SEQ ID NO: 456), BRRBRRFQILYRBHBH (SEQ ID NO: 457), RRBRRFQILYRBHBH (SEQ ID NO: 458), BRRFQILYRBHBH (SEQ ID NO: 459), RRFQILYRBHBH (SEQ ID NO: 460), RBRRBRRFQILYRBHB (SEQ ID NO: 461), RBRRBRRFQILYRBH (SEQ ID NO: 462), RBRRBRRFQILYRB (SEQ ID NO: 463), RBRRBRRFQILYR (SEQ ID NO: 464), HRBHRBHRBFQILYHRBRH (SEQ ID NO: 465), HRBHRBHRBFQILYHRBHRBHR (SEQ ID NO: 466), HRBHRBFQILYHRBHR (SEQ ID NO: 467), HRBFQILYHR (SEQ ID NO: 468), RBRBRBFQILYRBRBR (SEQ ID NO: 469), RBRBFQILYRBR (SEQ ID NO: 470), RBFQILYR (SEQ ID NO: 471), HRHRHRFQILYHRHRHR (SEQ ID NO: 472), HRHRFQILYHRHR (SEQ ID NO: 473), HRFQILYHR (SEQ ID NO: 474), RRRRRRFQILY (SEQ ID NO: 475), FQILYRRRRRR (SEQ ID NO: 476), RRRRRRFQILYRRRRRR (SEQ ID NO: 477), RBRRBRFQILYBR (SEQ ID NO: 478), and RBRRBRFQILY (SEQ ID NO: 479). In another aspect, the conjugate is of a peptide and a therapeutic or diagnostic molecule covalently bonded (i.e., directly) or linked via a non-cationic linker (e.g., via a linker including a total of one amino acid or via an aliphatic dicarboxylic acid linker) to the peptide, the peptide being rBrrBrfqilyBrBr (SEQ ID NO: 510), where lower case letters indicate D-amino acids.

In some embodiments, the peptide of the conjugate is (RBRRBRFQILYBR (SEQ ID NO: 478)). In some embodiments, the peptide of the conjugate is (RBRRBRFQILY (SEQ ID NO: 479). In some embodiments, the peptide of the conjugate is (RBRRBRRFQILY (SEQ ID NO: 456). In some embodiments, the peptide of the conjugate is (RRFQILYRBHBH (SEQ ID NO: 460)).

In another aspect, the conjugate is of a peptide and a therapeutic or diagnostic molecule covalently bonded (i.e., directly) or linked via a non-cationic linker (e.g., via a linker including a total of one amino acid or via an aliphatic dicarboxylic acid linker) to the peptide, the peptide being selected from the group consisting of: RBRRFQILYRBHBHB (SEQ ID NO: 481), RBRFQILYRBHBHB (SEQ ID NO: 482), RBFQILYRBHBHB (SEQ ID NO: 483), RFQILYRBHBHB (SEQ ID NO: 484), FQILYRBHBHB (SEQ ID NO: 485), RBRRBRRFQILYBHBHB (SEQ ID NO: 452), RBRRBRRFQILYHBHB (SEQ ID NO: 486), RBRRBRRFQILYBHB (SEQ ID NO: 487), RBRRBRRFQILYHB (SEQ ID NO: 488), RBRRBRRFQILYB (SEQ ID NO: 489), RBRRBRRFQILYE (SEQ ID NO: 416), RBRRBRRFQILY (SEQ ID NO: 456), BRRBRRFQILYRBHBHB (SEQ ID NO: 490), RRBRRFQILYRBHBHB (SEQ ID NO: 491), BRRFQILYRBHBHB (SEQ ID NO: 492), RRFQILYRBHBHB (SEQ ID NO: 493), RRFQILYRBHBHE (SEQ ID NO: 417), RBRRBRRFQILYRBHBB (SEQ ID NO: 494), RBRRBRRFQILYRBHB (SEQ ID NO: 461), RBRRBRRFQILYRBB (SEQ ID NO: 495), RBRRBRRFQILYRB (SEQ ID NO: 463), HRBHRBHRBFQILYHRBRHB (SEQ ID NO: 496), HRBHRBHRBFQILYHRBHRBHRB (SEQ ID NO: 497), HRBHRBFQILYHRBHRB (SEQ ID NO: 498), HRBFQILYHRB (SEQ ID NO: 499), RBRBRBFQILYRBRBRB (SEQ ID NO: 500), RBRBFQILYRBRB (SEQ ID NO: 501), RBFQILYRB (SEQ ID NO: 502), HRHRHRFQILYHRHRHRB (SEQ ID NO: 503), HRHRFQILYHRHRB (SEQ ID NO: 504), HRFQILYHRB (SEQ ID NO: 505), RRRRRRFQILYB (SEQ ID NO: 506), FQILYRRRRRRB (SEQ ID NO: 507), RRRRRRFQILYRRRRRRB (SEQ ID NO: 508), RBRRBRFQILYBRE (SEQ ID NO: 415), and RBRRBRFQILYE (SEQ ID NO: 509). In another aspect, the conjugate is of a peptide and a therapeutic or diagnostic molecule covalently bonded (i.e., directly) or linked via a non-cationic linker (e.g., via a linker including a total of one amino acid or via an aliphatic dicarboxylic acid linker) to the peptide, the peptide being rBrrBrfqilyBrBre (SEQ ID NO: 405), where lower case letters indicate D-amino acids. In another aspect, the conjugate comprises a peptide as set forth previously in this paragraph, but with the C-terminal amino acid removed or removed and replaced with a linker, e.g., as described herein (e.g., a B or an E linker).

In some embodiments, the peptide of the conjugate is DPep1.9b-del2 (RBRRBRFQILYBRE) (SEQ ID NO: 415). In some embodiments, the peptide of the conjugate is Dpep1.9b-del4 (RBRRBRFQILYE (SEQ ID NO: 509)). In some embodiments, the peptide of the conjugate is peptide E5-E (RBRRBRRFQILYE (SEQ ID NO: 416)). In some embodiments, the peptide of the conjugate is peptide G5-E (RRFQILYRBHBHE (SEQ ID NO: 417)).

In some embodiments, the peptide is bonded to the rest of the conjugate through its N-terminus. In some embodiments, the C-terminus of the peptide is amidated, i.e., terminated with —NH₂. In some embodiments, the peptide is bonded to the rest of the conjugate through its C-terminus. In some embodiments, the peptide is acylated at its N-terminus. In some embodiments, the peptide is covalently bonded (i.e., directly) to the therapeutic or diagnostic molecule (e.g., oligonucleotide). In some embodiments, the peptide is covalently linked to the therapeutic or diagnostic molecule (e.g., oligonucleotide) via a linker including a total of one amino acid. In some embodiments, the linker is selected from the group consisting of glutamic acid (e.g., linked via its gamma carboxyl group), beta-alanine, glycine, delta-aminovaleric acid, and gamma-aminobutyric acid. In some embodiments, the peptide is covalently bonded to the therapeutic or diagnostic molecule (e.g., oligonucleotide) via an aliphatic dicarboxylic acid linker (e.g., succinyl).

In some embodiments, the linker is of the following structure:

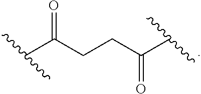 [SMILES: *C(=O)CCC(*)=O]

In some embodiments, the linker is of the following structure:

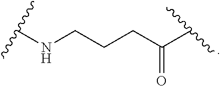 [SMILES: *NCCCC(*)=O]

In some embodiments, the linker is of the following structure:

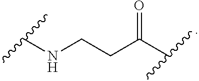 [SMILES: *NCCC(*)=O]

In some embodiments, the linker is of the following structure:

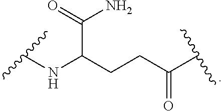 [SMILES: *NC(CCC(*)=O)C(N)=O]

In some embodiments, the linker is of the following structure:

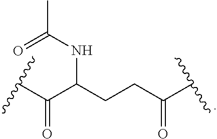 [SMILES: *C(=O)CCC(NC(C)=O)C(*)=O]

In some embodiments, the conjugate is of the following structure:

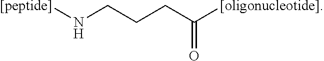

In some embodiments, the conjugate is of the following structure:

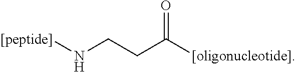

In some embodiments, the conjugate is of the following structure:

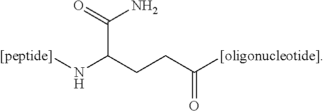

In some embodiments, the conjugate is of the following structure:

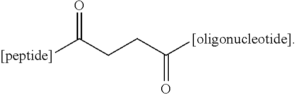

In some embodiments, the conjugate is of the following structure:

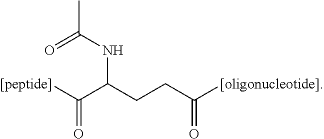

In some embodiments, the therapeutic or diagnostic molecule is an oligonucleotide, which optionally is bonded to the linker or the peptide at its 3′ terminus.

In some embodiments, the oligonucleotide comprises a sequence that is complementary to a target sequence, the targeting of which by the oligonucleotide alters processing or recognition of the target sequence, wherein optionally the targeting can be used to treat, improve, or prevent one or more symptoms of a genetic disorder.

In some embodiments, the genetic disorder is a neuromuscular disorder, which optionally is selected from the group consisting of muscular dystrophy (e.g., DMD, BMD, or FSHD), DM1, DM2, and SMA.

In some embodiments, the target sequence is within a transcript of the DMD, DUX4, DMPK, CNBP, or SMN2 gene.

In some embodiments, the genetic disorder is a neurologic disorder, which optionally is selected from the group consisting of CMT1a, CMT2a, and FA.

In some embodiments, the genetic disorder affects other organ systems of the body, which optionally includes FCD.

In some embodiments, the target sequence is within a transcript of the PMP22, MFN2, TF4, or FXN gene.

In some embodiments, the oligonucleotide includes at least 12 contiguous nucleobases that are complementary to a target exon sequence in a transcript of the DMD gene (e.g., a human dystrophin gene). In some embodiments, the target sequence is disposed within or proximal to any one of exons 8-55 of the gene transcript (e.g., exon 8, 23, 44, 45, 50, 51, 52, 53, or 55) (e.g., a human dystrophin gene).

In some embodiments, the oligonucleotide comprises or consists of a sequence of a Table herein (Table 1, Table 2, Table 3, Table 4, Table 5, or Table 8), wherein optionally one or more (e.g., all) uracils are replaced with thymines or one or more (e.g., all) thymines are replaced with uracils.

In some embodiments, the target sequence includes a splice site for exon 45 or is disposed within 150 nucleobases of a splice site for exon 45.

In some embodiments, the oligonucleotide includes at least 12 contiguous nucleobases from any one sequence in Table 1 and thymine-substituted versions thereof. In some embodiments, the oligonucleotide includes any one sequence in Table 1 or a thymine-substituted version thereof. In some embodiments, the sequence in Table 1 is:

(SEQ ID NO: 276)
5′-GCTGCCCAATGCCATCCTGGAGTTCCTGTAA-3′.

In some embodiments, the sequence in Table 1 is:

(SEQ ID NO: 275)
5′-CAATGCCATCCTGGAGTTCCTG-3′.

In some embodiments, the sequence in Table 1 is selected from the group consisting of:

(SEQ ID NO: 270)
5′-TTGCCGCTGCCCAATGCCATCCTGGAGTTC-3′,
(SEQ ID NO: 271)
5′-CAGTTTGCCGCTGCCCAATGCCATCCTGGA-3′,
(SEQ ID NO: 272)
5′-CCAAUGCCAUCCUGGAGUUCCUGUAA-3′,
(SEQ ID NO: 273)
5′-CCAATGCCATCCTGGAGTTCCTGTA-3′,
and
(SEQ ID NO: 274)
5′-CTGACAACAGTTTGCCGCTGCCCAA-3′.

In some embodiments, the target sequence includes a splice site for exon 51 or is disposed within 150 nucleobases of a splice site for exon 51.

In some embodiments, the oligonucleotide includes at least 12 contiguous nucleobases from any one sequence in Table 2 and thymine-substituted versions thereof.

In some embodiments, the oligonucleotide includes any one sequence in Table 2 or a thymine-substituted version thereof.

In some embodiments, the sequence in Table 2 is:

(SEQ ID NO: 282)
5′-CUCCAACAUCAAGGAAGAUGGCAUUUCUAG-3′.

In some embodiments, the sequence in Table 2 is:

(SEQ ID NO: 287)
5′-CTCCAACATCAAGGAAGATGGCATTTCTAG-3′.

In some embodiments, the target sequence includes a splice site for exon 53 or is disposed within 150 nucleobases of a splice site for exon 53.

In some embodiments, the oligonucleotide includes at least 12 contiguous nucleobases from any one sequence in Table 3.

In some embodiments, the oligonucleotide includes any one sequence in Table 3.

In some embodiments, the sequence in Table 3 is:

(SEQ ID NO: 316)
5′-CCTCCGGTTCTGAAGGTGTTCT-3′.

In some embodiments, the sequence in Table 3 is:

(SEQ ID NO: 325)
5′-GTTGCCTCCGGTTCTGAAGGTGTTC-3′.

In some embodiments, the sequence in Table 3 is selected from the group consisting of:

(SEQ ID NO: 327)
5′-CTGTTGCCTCCGGTTCTGAAGGTGTTCTTG-3′,
(SEQ ID NO: 328)
5′-CAACTGTTGCCTCCGGTTCTGAAGGTGTTC-3′,
(SEQ ID NO: 329)
5′-TTGCCTCCGGTTCTGAAGGTGTTCTTGTAC-3′,
(SEQ ID NO: 330)
5′-CTGAAGGTGTTCTTGTACTTCATCC-3′,
and
(SEQ ID NO: 331)
5′-CATTCAACTGTTGCCTCCGGTTCTGAAGGTG-3′.

In some embodiments, the target sequence comprises a splice site for exon 44 or is disposed within 150 nucleobases of a splice site for exon 44. In some embodiments, the oligonucleotide comprises at least 12 contiguous nucleobases from any one sequence in Table 4. In some embodiments, the oligonucleotide comprises any one sequence in Table 4.

In some embodiments, the sequence in Table 4 is selected from the group consisting of:

(SEQ ID NO: 335)
5′-TGAAAACGCCGCCATTTCTCAACAGATCTG-3′,
(SEQ ID NO: 336)
5′-CATAATGAAAACGCCGCCATTTCTCAACAG-3′,
(SEQ ID NO: 337)
5′-TGTTCAGCTTCTGTTAGCCACTGATTAAAT-3′,
(SEQ ID NO: 338)
5′-CGCCGCCATTTCTCAACAG-3′,
and
(SEQ ID NO: 339)
5′-ATCTGTCAAATCGCCTGCAG-3′.

In some embodiments, the sequence is 5′-GGCCAAACCTCGGCTTACCTGAAAT-3′ (SEQ ID NO: 288).

In some embodiments, the splice site is an acceptor splice site.

In some embodiments, the splice site is a donor splice site.

In some embodiments, the oligonucleotide comprises at least 9 contiguous nucleobases complementary to a CUG repeat sequence. In some embodiments, the oligonucleotide has a sequence selected from the group consisting of 5′-[CAG]ₙ-3′, 5′-[AGC]ₙ-3′, and 5′-[GCA]ₙ-3′, wherein n is an integer from 5 to 8. In some embodiments, the oligonucleotide has a sequence selected from the group consisting of 5′-[CAG]₅-3′ (SEQ ID NO: 340), 5′-[CAG]₆-3′ (SEQ ID NO: 341), 5′-[CAG]₇-3′ (SEQ ID NO: 342), 5′-[CAG]₈-3′ (SEQ ID NO: 343), 5′-[AGC]₅-3′ (SEQ ID NO: 344), 5′-[AGC]₆-3′ (SEQ ID NO: 345), 5′-[AGC]₇-3′ (SEQ ID NO: 346), 5′-[AGC]₈-3′ (SEQ ID NO: 347), 5′-[GCA]₅-3′ (SEQ ID NO: 348), 5′-[GCA]₆-3′ (SEQ ID NO: 349), 5′-[GCA]₇-3′ (SEQ ID NO: 350), and 5′-[GCA]₈-3′ (SEQ ID NO: 351).

In some embodiments, the oligonucleotide comprises at least 8 contiguous nucleobases complementary to a CCUG repeat sequence. In some embodiments, the oligonucleotide has a sequence selected from the group consisting of 5′-[CAGG]ₙ-3′, 5′-[AGGC]ₙ-3′, 5′-[GGCA]ₙ-3′, 5′-[GCAG]ₙ-3′, wherein n is an integer from 4 to 8. In some embodiments, the oligonucleotide has a sequence selected from the group consisting of 5′-[CAGG]₄-3′ (SEQ ID NO: 352), 5′-[CAGG]₅-3′ (SEQ ID NO: 353), 5′-[CAGG]₆-3′ (SEQ ID NO: 354), 5′-[CAGG]₇-3′ (SEQ ID NO: 355), 5′-[CAGG]ₙ-3′ (SEQ ID NO: 356), 5′-[AGGC]₄-3′(SEQ ID NO: 357), 5′-[AGGC]₅-3′ (SEQ ID NO: 358), 5′-[AGGC]₆-3′ (SEQ ID NO: 359), 5′-[AGGC]₇-3′ (SEQ ID NO: 360), 5′-[AGGC]₈-3′ (SEQ ID NO: 361), 5′-[GGCA]₄-3′ (SEQ ID NO: 362), 5′-[GGCA]₅-3′ (SEQ ID NO: 363), 5′-[GGCA]₆-3′ (SEQ ID NO: 364), 5′-[GGCA]₇-3′ SEQ ID NO: 365), 5′-[GGCA]₈-3′ (SEQ ID NO: 366), 5′-[GCAG]₄-3′ (SEQ ID NO: 367), 5′-[GCAG]₅-3′ (SEQ ID NO: 368), 5′-[GCAG]₆-3′ (SEQ ID NO: 369), 5′-[GCAG]₇-3′ (SEQ ID NO: 370), and 5′-[GCAG]₈-3′ (SEQ ID NO: 371).

In some embodiments, the oligonucleotide comprises at least 12 contiguous nucleobases that are complementary to a target sequence in a transcript of the SMN2 gene. In some embodiments, the target sequence is disposed in SMN2 intron 7. In some embodiments, the oligonucleotide comprises any one sequence in Table 5 and thymine-substituted versions thereof.

In some embodiments, the oligonucleotide comprises any one sequence in Table 8 and thymine-substituted versions thereof.

In some embodiments, the oligonucleotide includes the following group as its 5′ terminus:

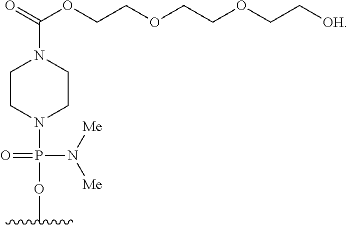 [SMILES: *OP(=O)(N(C)C)N1CCN(C(=O)OCCOCCOCCO)CC1]

In some embodiments, the oligonucleotide includes the following group as its 5′ terminus:

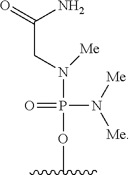 [SMILES: *OP(=O)(N(C)C)N(C)CC(N)=O]

In some embodiments, the oligonucleotide includes hydroxyl as its 5′ terminus.

In another aspect, the invention provides a pharmaceutical composition including a conjugate described herein and a pharmaceutically acceptable excipient.

In yet another aspect, the invention provides a method of treating a subject having a genetic disorder, the method comprising administering to the subject a therapeutically effective amount of a conjugate or a pharmaceutical composition described herein.

In some embodiments, the genetic disorder is a neuromuscular disorder, which optionally is selected from the group consisting of muscular dystrophy (e.g., DMD, BMD, or FSHD), DM1, DM2, and SMA.

In some embodiments, the target sequence is within a transcript of the DMD, DMPK, CNBP, SMN2, or DUX4 gene.

In some embodiments, the genetic disorder is a neurologic disorder, which optionally is selected from the group consisting of CMT1a, CMT2a, and FA.

In some embodiments, the genetic disorder affects other organ systems of the body, which optionally includes FCD.

In some embodiments, the target sequence is within a transcript of the PMP22, MFN2, TF4, or RXN gene.

In some embodiments, the method is for treating a subject having DMD, BMD, SMA, DM1, or DM2, and the method includes administering to the subject a therapeutically effective amount of the conjugate described herein or the pharmaceutical composition described herein, wherein the conjugate is for treating DMD or BMD, the conjugate is for treating DM1, the conjugate is for treating DM2, and the conjugate is for treating SMA.

In some embodiments, the subject has DMD. In some embodiments, the subject has DM1. In some embodiments, the subject has DM2. In some embodiments, the subject has SMA.

Preferably, the oligonucleotide is a morpholino (more preferably, a morpholino with all morpholino internucleoside linkages being —P(O)(Nme₂)O—).

In some embodiments, the oligonucleotide is a phosphorothioate (PS) oligonucleotide.

In some embodiments, the oligonucleotide is a 2′-O-alkyl oligoribonucleotide, e.g., a 2′-O-methyl oligoribonucleotide.

In some embodiments, the oligonucleotide is a 2′-O-alkyl phosphorothioate, e.g., a 2′-O-methyl phosphorothioate.

In some embodiments, the oligonucleotide is a peptide nucleic acid (PNA).

In another aspect, the invention provides a peptide including a total of one hydrophobic domain and a total of one cationic domain, the cationic domain including at least 1 cationic amino acid residue, and the hydrophobic domain including at least 3 amino acid residues, provided that the peptide includes a total of 5 to 40 (e.g., 6 to 40 or 7 to 40) amino acid residues and does not include any artificial amino acid residues. In some embodiments, the amino acid residues for each cationic domain are selected from the group consisting of arginine, histidine, and beta-alanine.

In yet another aspect, the invention provides a peptide including a total of one hydrophobic domain and a total of one or two cationic domains flanking the hydrophobic domain, each cationic domain including at least one cationic amino acid residue, at least one of the cationic domains including a total of one cationic amino acid residue, and the hydrophobic domain including at least 3 amino acid residues, provided that the peptide includes a total of 5 to 40 (e.g., 6 to 40 or 7 to 40) amino acid residues and does not include any artificial amino acid residues. In some embodiments, the amino acid residues for each cationic domain are selected from the group consisting of arginine, histidine, and beta-alanine. In some embodiments, at least one cationic domain includes three or fewer amino acid residues.

In still another aspect, the invention provides a peptide including a total of one hydrophobic domain and a total of one or two cationic domains flanking the hydrophobic domain, each cationic domain including at least 1 cationic amino acid residue, all cationic domains collectively including a total of five or fewer cationic amino acid residue, and the hydrophobic domain including at least 3 amino acid residues, provided that the peptide includes a total of 5 to 40 (e.g., 6 to 40 or 7 to 40) amino acid residues and does not include any artificial amino acid residues. In some embodiments, the amino acid residues for each cationic domain are selected from the group consisting of arginine, histidine, and beta-alanine. In some embodiments, at least one cationic domain includes three or fewer amino acid residues.

In a further aspect, the invention provides a peptide including a total of one hydrophobic domain and a total of one or two cationic domains flanking the hydrophobic domain, each cationic domain including at least 1 cationic amino acid residue, at least one third of amino acid residues in the N-terminal cationic domain are histidines, and the hydrophobic domain including at least 3 amino acid residues, provided that the peptide includes a total of 5 to 40 (e.g., 6 to 40 or 7 to 40) amino acid residues and does not include any artificial amino acid residues. In some embodiments, the amino acid residues for each cationic domain are selected from the group consisting of arginine, histidine, and beta-alanine. In some embodiments, at least one cationic domain includes three or fewer amino acid residues.

In some embodiments, each cationic domain includes at least 40%, at least 45%, or at least 50% cationic amino acids. In some embodiments, each cationic domain includes a majority of cationic amino acids, preferably at least at least 55%, at least 60%, at least 65% at least 70%, at least 75%, at least 80%, at least 85%, at least 90%, at least 95% cationic amino acids. In some embodiments, each cationic domain includes arginine, histidine, beta-alanine, hydroxyproline and/or serine residues, preferably wherein each cationic domain consists of arginine, histidine, beta-alanine, hydroxyproline and/or serine residues, provided at least one arginine or histidine is present. In some embodiments, each cationic domain is arginine rich and/or histidine rich, preferably each cationic domain includes at least 40%, at least 45%, at least 50%, at least 55%, at least 60%, at least 60%, at least 65%, or least 70% arginine and/or histidine residues.

In some embodiments, at least one cationic domain is selected from the group consisting of. RBRR (SEQ ID NO: 419), RBR, RB, R, RBRRBRR (SEQ ID NO: 420), RRBRR (SEQ ID NO: 421), BRR, RR, HRBHRBHRB (SEQ ID NO: 422), HRBHRB (SEQ ID NO: 423), HRB, RBRBRB (SEQ ID NO: 424), RBRB (SEQ ID NO: 425), RB, HRHRHR (SEQ ID NO: 426), HRHR (SEQ ID NO: 427), HR, RRRRRR (SEQ ID NO: 428), RBRRBR SEQ ID NO: 429), RBHBHB (SEQ ID NO: 430), RBHBH (SEQ ID NO: 431), BHBHB (SEQ ID NO: 432), BHBH (SEQ ID NO: 433), HBHB (SEQ ID NO: 438), HBH, BHB, BH, HB, H, RBHBHE (SEQ ID NO: 435), RBHBB (SEQ ID NO: 436), RBHB (SEQ ID NO: 437), RBB, HRBRHB (SEQ ID NO: 438), HRBHRBHR (SEQ ID NO: 439), HRBHR (SEQ ID NO: 440), HRB, RBRBR (SEQ ID NO: 441), HRHRHRB (SEQ ID NO: 442), HRHRB (SEQ ID NO: 443), HRBRH (SEQ ID NO: 444), HRB, RRRRRRB (SEQ ID NO: 445), BRE, and BR, e.g., RBRR (SEQ ID NO: 419), RBR, RB, R, RBRRBRR (SEQ ID NO: 420), RRBRR (SEQ ID NO: 421), BRR, RR, HRBHRBHRB (SEQ ID NO: 422), HRBHRB (SEQ ID NO: 423), HRB, RBRBRB (SEQ ID NO: 424), RBRB (SEQ ID NO: 425), RB, HRHRHR (SEQ ID NO: 426), HRHR (SEQ ID NO: 427), HR, RRRRRR (SEQ ID NO: 428), RBRRBR (SEQ ID NO: 429), RBHBH (SEQ ID NO: 431), BHBH (SEQ ID NO: 433), HBH, BH, H, RBHB (SEQ ID NO: 437), HRBRH (SEQ ID NO: 444), HRBHRBHR (SEQ ID NO: 439), HRBHR (SEQ ID NO: 440), RBRBR (SEQ ID NO: 441), and BR. In some embodiments, at least one cationic domain is selected from the group consisting of RBR, RBRB (SEQ ID NO: 425), RB, R, RBRRBRR (SEQ ID NO: 420), BRR, BR, RR, HRBHRBHRB

(SEQ ID NO: 422), HRBHRB (SEQ ID NO: 423), HRBHRBHR (SEQ ID NO: 439), HRBHR (SEQ ID NO: 440), HRB, HRHRHR (SEQ ID NO: 426), HR, RRRRRR (SEQ ID NO: 428), RRRRRRB (SEQ ID NO: 445), RBHBH (SEQ ID NO: 431), BH, BHB, H, HB, RBH, and RBHB (SEQ ID NO: 437), e.g., RBR, R, RBRRBRR (SEQ ID NO: 420), BRR, BR, RR, HRBHRBHR (SEQ ID NO: 439), HRBHR (SEQ ID NO: 440), HRHRHR (SEQ ID NO: 426), HR, RRRRRR (SEQ ID NO: 428), RBHBH (SEQ ID NO: 431), BH, H, and RBH.

In some embodiments, each hydrophobic domain has a length of between 3-6 amino acids, preferably each hydrophobic domain has a length of 5 amino acids.

In some embodiments, each hydrophobic domain includes a majority of hydrophobic amino acid residues, preferably each hydrophobic domain includes at least 70%, at least 75%, at least 80%, at least 85%, at least 90%, at least 95%, or 100% hydrophobic amino acids.

In some embodiments, each hydrophobic domain includes phenylalanine, leucine, isoleucine, tyrosine, tryptophan, proline, and glutamine residues; preferably wherein each hydrophobic domain consists of phenylalanine, leucine, isoleucine, tyrosine, tryptophan, proline, and/or glutamine residues, provided at least one of phenylalanine, leucine, isoleucine, tyrosine, and tryptophan is present.

In some embodiments, the hydrophobic domain is FQILY (SEQ ID NO: 446).

In yet a further aspect, the invention provides a peptide selected from the group consisting of: RBRRFQILYRBHBH (SEQ ID NO: 447), RBRFQILYRBHBH (SEQ ID NO: 448), RBFQILYRBHBH (SEQ ID NO: 449), RFQILYRBHBH (SEQ ID NO: 450), FQILYRBHBH (SEQ ID NO: 451), RBRRBRRFQILYBHBHB (SEQ ID NO: 452), RBRRBRRFQILYHBH (SEQ ID NO: 453), RBRRBRRFQILYBH (SEQ ID NO: 454), RBRRBRRFQILYH (SEQ ID NO: 455), RBRRBRRFQILY (SEQ ID NO: 456), BRRBRRFQILYRBHBH (SEQ ID NO: 457), RRBRRFQILYRBHBH (SEQ ID NO: 458), BRRFQILYRBHBH (SEQ ID NO: 459), RRFQILYRBHBH (SEQ ID NO: 460), RBRRBRRFQILYRBHB (SEQ ID NO: 461), RBRRBRRFQILYRBH (SEQ ID NO: 462), RBRRBRRFQILYRB (SEQ ID NO: 463), RBRRBRRFQILYR (SEQ ID NO: 464), HRBHRBHRBFQILYHRBRH (SEQ ID NO: 465), HRBHRBHRBFQILYHRBHRBHR (SEQ ID NO: 466), HRBHRBFQILYHRBHR (SEQ ID NO: 467), HRBFQILYHR (SEQ ID NO: 468), RBRBRBFQILYRBRBR (SEQ ID NO: 469), RBRBFQILYRBR (SEQ ID NO: 470), RBFQILYR (SEQ ID NO: 471), HRHRHRFQILYHRHRHR (SEQ ID NO: 472), HRHRFQILYHRHR (SEQ ID NO: 473), HRFQILYHR (SEQ ID NO: 474), RRRRRRFQILY (SEQ ID NO: 475), FQILYRRRRRR (SEQ ID NO: 476), RRRRRRFQILYRRRRRR (SEQ ID NO: 477), RBRRBRFQILYBR (SEQ ID NO: 478), and RBRRBRFQILY (SEQ ID NO: 479). In another aspect, the invention provides a peptide being rBrrBrfqilyBrBr (SEQ ID NO: 510), where lower case letters indicate D-amino acids.

In some embodiments, the peptide is (RBRRBRFQILYBR (SEQ ID NO: 478)). In some embodiments, the peptide is (RBRRBRFQILY (SEQ ID NO: 479)). In some embodiments, the peptide is (RBRRBRRFQILY (SEQ ID NO: 456)). In some embodiments, the peptide is (RRFQILYRBHBH (SEQ ID NO: 460)).

In yet a further aspect, the invention provides a peptide selected from the group consisting of: RBRRFQILYRBHBHB (SEQ ID NO: 481), RBRFQILYRBHBHB (SEQ ID NO: 482), RBFQILYRBHBHB (SEQ ID NO: 483), RFQILYRBHBHB (SEQ ID NO: 484), FQILYRBHBHB (SEQ ID NO: 485), RBRRBRRFQILYBHBHB (SEQ ID NO: 452), RBRRBRRFQILYHBHB (SEQ ID NO: 486), RBRRBRRFQILYBHB (SEQ ID NO: 487), RBRRBRRFQILYHB (SEQ ID NO: 488), RBRRBRRFQILYB (SEQ ID NO: 489), RBRRBRRFQILYE (SEQ ID NO: 416), RBRRBRRFQILY (SEQ ID NO: 456), BRRBRRFQILYRBHBHB (SEQ ID NO: 490), RRBRRFQILYRBHBHB (SEQ ID NO: 491), BRRFQILYRBHBHB (SEQ ID NO: 492), RRFQILYRBHBHB (SEQ ID NO: 493), RRFQILYRBHBHE (SEQ ID NO: 417), RBRRBRRFQILYRBHBB (SEQ ID NO: 494), RBRRBRRFQILYRBHB (SEQ ID NO: 461), RBRRBRRFQILYRBB (SEQ ID NO: 495), RBRRBRRFQILYRB (SEQ ID NO: 463), HRBHRBHRBFQILYHRBRHB (SEQ ID NO: 496), HRBHRBHRBFQILYHRBHRBHRB (SEQ ID NO: 497), HRBHRBFQILYHRBHRB (SEQ ID NO: 498), HRBFQILYHRB (SEQ ID NO: 499), RBRBRBFQILYRBRBRB (SEQ ID NO: 500), RBRBFQILYRBRB (SEQ ID NO: 501), RBFQILYRB (SEQ ID NO: 502), HRHRHRFQILYHRHRHRB (SEQ ID NO: 503), HRHRFQILYHRHRB (SEQ ID NO: 504), HRFQILYHRB (SEQ ID NO: 505), RRRRRRFQILYB (SEQ ID NO: 506), FQILYRRRRRRB (SEQ ID NO: 507), RRRRRRFQILYRRRRRRB (SEQ ID NO: 508), RBRRBRFQILYBRE (SEQ ID NO: 415), and RBRRBRFQILYE (SEQ ID NO: 509). In some embodiments, the peptide is Dpep1.9b-del2 (RBRRBRFQILYBRE) (SEQ ID NO: 415). In some embodiments, the peptide is Dpep1.9b-del4 (RBRRBRFQILYE) (SEQ ID NO: 509). In some embodiments, the peptide is peptide E5-E (RBRRBRRFQILYE) (SEQ ID NO: 416). In some embodiments, the peptide is peptide G5-E (RRFQILYRBHBHE) (SEQ ID NO: 417). In another aspect, the invention provides a peptide being rBrrBrfqilyBrBre (SEQ ID NO: 405), where lower case letters indicate D-amino acids. In another aspect, the peptide is as set forth previously in this paragraph, but with the C-terminal amino acid removed or removed and replaced with a linker, e.g., as described herein (e.g., a B or an E linker).

In another aspect, the invention provides a peptide comprising or consisting of the sequence of SEQ ID NO: 415, 509, 416, 417, 418, 402, 512, 403, 404, 405, 478, 479, 456, or 460. In a further aspect, the invention provides a conjugate comprising such a peptide. In some embodiments, the conjugate comprises a therapeutic or diagnostic molecule (e.g., an oligonucleotide, such as a morpholino). In some embodiments, the sequence of the oligonucleotide is selected from Table 1, 2, 3, 4, 5, or 8.

### Definitions

References to “X” throughout denote any form of the amino acid aminohexanoic acid, such as 6-aminohexanoic acid.

References to “B” throughout denote the amino acid beta-alanine.

Refences to “[Hyp]” throughout denote the amino acid hydroxyproline.

References to “Ac” throughout denote an acetyl group (CH₃—C(O)—).

References to other capital letters throughout denote the relevant amino acid residues in accordance with the accepted alphabetic amino acid code.

The term “alkyl,” as used herein, refers to a straight or branched chain hydrocarbon group containing a total of one to twenty carbon atoms, unless otherwise specified (e.g., (1-6C) alkyl, (1-4C) alkyl, (1-3C) alkyl, or (1-2C) alkyl). Non-limiting examples of alkyls include methyl, ethyl, 1-methylethyl, propyl, 1-methylbutyl, 1-ethylbutyl, etc. References to individual alkyl groups such as “propyl” are specific for the straight chain version only, and references to individual branched chain alkyl groups such as “isopropyl” are specific for the branched chain version only.

The term “alkenyl”, as used herein, refers to an aliphatic group containing having one, two, or three carbon-carbon double bonds and containing a total of two to twenty carbon atoms, unless otherwise specified (e.g., (2-6C) alkenyl, (2-4C) alkenyl, or (2-3C) alkenyl). Non-limiting examples of alkenyl include vinyl, allyl, homoallyl, isoprenyl, etc. Unless otherwise specified, alkenyl may be optionally substituted by one, two, three, four, or five groups selected from the group consisting of carbocyclyl, aryl, heterocyclyl, heteroaryl, oxo, halogen, and hydroxyl.

The term “alkynyl”, as used herein, refers to an aliphatic group containing one, two, or three carbon-carbon triple bonds and containing a total of two to twenty carbon atoms, unless otherwise specified (e.g., (2-6C) alkynyl, (2-4C) alkynyl, or (2-3C) alkynyl). Non-limiting examples of alkynyl include ethynyl, propargyl, homopropargyl, but-2-yn-1-yl, 2-methyl-prop-2-yn-1-yl, etc. Unless otherwise specified, alkynyl may be optionally substituted by one, two, three, four, or five groups selected from the group consisting of carbocyclyl, aryl, heterocyclyl, heteroaryl, oxo, halogen, and hydroxyl.

By “arginine rich,” it is meant that at least 40% of the cationic domain is formed of arginine residues.

The term “artificial amino acid,” as used herein, refers to an abiogenic amino acid (e.g., non-proteinogenic). For example, artificial amino acids may include synthetic amino acids, modified amino acids (e.g., those modified with sugars), non-natural amino acids, man-made amino acids, spacers, and non-peptide bonded spacers. For the avoidance of doubt, aminohexanoic acid (X) is an artificial amino acid in the context of the present invention. For the avoidance of doubt, beta-alanine (B) and hydroxyproline (Hyp) occur in nature and therefore are not artificial amino acids in the context of the present invention but are natural amino acids. Artificial amino acids may include, for example, 6-aminohexanoic acid (X), tetrahydroisoquinoline-3-carboxylic acid (TIC), 1-(amino)cyclohexanecarboxylic acid (Cy), 3-azetidine-carboxylic acid (Az), and 11-aminoundecanoic acid. For the avoidance of doubt, it is noted that the D enantiomer of a naturally occurring L-amino acid is not to be considered as an artificial amino acid.

The term “aryl,” as used herein, refers to a carbocyclic ring system containing one, two, or three rings, at least one of which is aromatic. An unsubstituted aryl contains a total of 6 to 14 carbon atoms. The term aryl includes both monovalent species and divalent species. Examples of aryl groups include, but are not limited to, phenyl, naphthyl, indanyl, and the like. In particular embodiments, an optionally substituted aryl is optionally substituted phenyl.

By “bridged ring systems,” as used herein, are meant ring systems in which two rings share more than two atoms, see for example Advanced Organic Chemistry, by Jerry March, 4th Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane, quinuclidine, etc.

The term “carbonyl,” as used herein, refers to a group of the following structure —C(O)—. Non-limiting examples of carbonyl groups include those found, e.g., in acetone, ethyl acetate, proteinogenic amino acids, acetamide, etc.

References made herein to “cationic” denote an amino acid or domain of amino acids having an overall positive charge at neutral pH, though positively charged and neutral forms may coexist at this pH. For example, at neutral pH, histidine is believed to have neutral and cationic forms in an equilibrium, as pKₐ of protonated histidine side chain is 6.3. Non-limiting examples of cationic amino acids include histidine, lysine, and arginine.

The term “(m-nC)” or “(m-nC) group” used alone or as a prefix, refers to a group having a total of m to n carbon atoms, when unsubstituted.

The term “complementary,” as used herein in reference to a nucleobase sequence, refers to the nucleobase sequence having a pattern of contiguous nucleobases that permits an oligonucleotide having the nucleobase sequence to hybridize to another oligonucleotide or nucleic acid to form a duplex structure under physiological conditions. Complementary sequences include Watson-Crick base pairs formed from natural and/or modified nucleobases. Complementary sequences can also include non-Watson-Crick base pairs, such as wobble base pairs (guanosine-uracil, hypoxanthine-uracil, hypoxanthine-adenine, and hypoxanthine-cytosine) and Hoogsteen base pairs.

The term “cycloalkyl,” as used herein, refers to a saturated carbocyclic ring system containing one or two rings, and containing a total of 3 to 10 carbon atoms, unless otherwise specified. The two-ring cycloalkyls may be arranged as fused ring systems (two bridgehead carbon atoms are directly bonded to one another), bridged ring systems (two bridgehead carbon atoms are linked to one another via a covalent linker containing at least one carbon atom), and spiro-ring (two rings are fused at the same carbon atom) systems. Non-limiting examples of cycloalkyl include cyclopropyl, cyclobutyl, cyclopentyl, cyclohexyl, cycloheptyl, bicyclo[2.2.1]heptyl, etc.

The term “cycloalkenyl,” as used herein, refers to a non-aromatic, unsaturated, carbocyclic ring system containing one or two rings; containing one, two, or three endocyclic double bonds; and containing a total of 3 to 10 carbon atoms, unless otherwise specified. The two-ring cycloalkenyls may be arranged as fused ring systems (two bridgehead carbon atoms are directly bonded to one another), bridged ring systems (two bridgehead carbon atoms are linked to one another via a covalent linker containing at least one carbon atom), and spiro-ring (two rings are fused at the same carbon atom) systems. Non-limiting examples of cycloalkenyl include cyclobutenyl, cyclopentenyl, cyclohexenyl, cycloheptenyl, 3-cyclohexen-1-yl, cyclooctenyl, etc.

“Dystrophin” is a rod-shaped cytoskeletal protein and a vital part of the dystrophin associated glycoprotein complex that connects the cytoskeleton of a muscle fiber to the surrounding extracellular matrix through the cell membrane. Dystrophin contains multiple functional domains. For instance, dystrophin contains an actin binding domain at about amino acids 14-240 and a central rod domain at about amino acids 253-3040. This large central domain is formed by 24 spectrin-like triple-helical elements of about 109 amino acids, which have homology to alpha-actinin and spectrin. The repeats are typically interrupted by four proline-rich non-repeat segments, also referred to as hinge regions. Repeats 15 and 16 are separated by an 18 amino acid stretch that appears to provide a major site for proteolytic cleavage of dystrophin. The sequence identity between most repeats ranges from 10-25%. One repeat contains three alpha-helices: 1, 2 and 3. Alpha-helices 1 and 3 are each formed by 7 helix turns, probably interacting as a coiled-coil through a hydrophobic interface. Alpha-helix 2 has a more complex structure and is formed by segments of four and three helix turns, separated by a glycine or proline residue. Each repeat is encoded by two exons, typically interrupted by an intron between amino acids 47 and 48 in the first part of alpha-helix 2. The other intron is found at different positions in the repeat, usually scattered over helix-3. Dystrophin also contains a cysteine-rich domain at about amino acids 3080-3360), including a cysteine-rich segment (i.e., 15 Cysteines in 280 amino acids) showing homology to the C-terminal domain of the slime mold (Dictyostelium discoideum) alpha-actinin. The carboxy-terminal domain is at about amino acids 3361-3685.

The amino-terminus of dystrophin binds to F-actin and the carboxy-terminus binds to the dystrophin-associated protein complex (DAPC) at the sarcolemma. The DAPC includes the dystroglycans, sarcoglycans, integrins and caveolin, and mutations in any of these components cause autosomally inherited muscular dystrophies. The DAPC is destabilized when dystrophin is absent, which results in diminished levels of the member proteins, and in turn leads to progressive fibre damage and membrane leakage. In various forms of muscular dystrophy, such as Duchenne muscular dystrophy (DMD) and Becker muscular dystrophy (BMD), muscle cells produce no dystrophin at all, or an altered and functionally defective form of dystrophin, respectively, mainly due to mutations in the gene sequence that lead to incorrect splicing. The predominant expression of the defective dystrophin protein, or the complete lack of dystrophin or a dystrophin-like protein, leads to rapid progression of muscle degeneration, as noted above. In this regard, a “defective” dystrophin protein may be characterized by the forms of dystrophin that are produced in certain subjects with DMD or BMD, as known in the art, or by the absence of detectable dystrophin.

An “exon” refers to a defined section of nucleic acid that encodes for a protein, or a nucleic acid sequence that is represented in the mature form of an RNA molecule after a portion of a pre-processed (or precursor) RNA has been removed by splicing. The mature RNA molecule can be a messenger RNA (mRNA) or a functional form of a non-coding RNA, such as rRNA or tRNA. The human dystrophin gene has about 79 exons.

“Exon skipping” refers generally to the process by which an entire exon, or a portion thereof, is removed from a given pre-processed RNA, and is thereby excluded from being present in the mature RNA, such as the mature mRNA that is translated into a protein. Hence, the portion of the protein that is otherwise encoded by the skipped exon is not present in the expressed form of the protein, typically creating an altered, though still functional, form of the protein. In certain embodiments, the exon being skipped is an aberrant exon from the human dystrophin gene, which may contain a mutation or other alteration in its sequence that otherwise causes aberrant splicing. In certain embodiments, the exon being skipped is exon 44, 45, 51, and/or 53 of the human dystrophin gene.

The term “halo” or “halogen,” as used herein, refer to fluoro, chloro, bromo, and iodo.

The term “heteroalkyl,” as used herein, refers to a straight or branched chain hydrocarbon group containing a total of one to thirty carbon atoms, unless otherwise specified, and at least one heteroatom that is oxygen, nitrogen, or sulfur. For example, a heteroalkyl may be (2-20C) heteroalkyl, (2-12C) heteroalkyl, or (2-10C) heteroalkyl. Non-limiting examples of alkyls include, e.g., PEG3.

By “histidine rich,” it is meant that at least 40% of the cationic domain is formed of histidine residues.

The terms “heteroaryl” or “heteroaromatic,” as used interchangeably herein, refer to a ring system containing one, two, or three rings, at least one of which is aromatic and containing one to four (e.g., one, two, or three) heteroatoms selected from the group consisting of nitrogen, oxygen, and sulfur. An unsubstituted heteroaryl group contains a total of one to nine carbon atoms. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example, a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example, a single heteroatom. In some embodiments, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1.2.3.4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1.2.3.4-tetrahydropyrido[2,3-b]pyrazinyl and 3,4-dihydro-2H-pyrido[3,2-b][1,4]oxazinyl. Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups. Examples of six membered heteroaryl groups include but are not limited to pyridyl, pyrazinyl, pyridazinyl, pyrimidinyl and triazinyl. A bicyclic heteroaryl group may be, for example, a group selected from: a benzene ring fused to a 5- or 6-membered ring containing 1, 2 or 3 ring heteroatoms; a pyridine ring fused to a 5- or 6-membered ring containing 1, 2 or 3 ring heteroatoms; a pyrimidine ring fused to a 5- or 6-membered ring containing 1 or 2 ring heteroatoms; a pyrrole ring fused to a 5- or 6-membered ring containing 1, 2 or 3 ring heteroatoms; a pyrazole ring fused to a 5- or 6-membered ring containing 1 or 2 ring heteroatoms; a pyrazine ring fused to a 5- or 6-membered ring containing 1 or 2 ring heteroatoms; an imidazole ring fused to a 5- or 6-membered ring containing 1 or 2 ring heteroatoms; an oxazole ring fused to a 5- or 6-membered ring containing 1 or 2 ring heteroatoms; an isoxazole ring fused to a 5- or 6-membered ring containing 1 or 2 ring heteroatoms; a thiazole ring fused to a 5- or 6-membered ring containing 1 or 2 ring heteroatoms; an isothiazole ring fused to a 5- or 6-membered ring containing 1 or 2 ring heteroatoms; a thiophene ring fused to a 5- or 6-membered ring containing 1, 2 or 3 ring heteroatoms; a furan ring fused to a 5- or 6-membered ring containing 1, 2 or 3 ring heteroatoms; a cyclohexyl ring fused to a 5- or 6-membered heteroaromatic ring containing 1, 2 or 3 ring heteroatoms; and a cyclopentyl ring fused to a 5- or 6-membered heteroaromatic ring containing 1, 2 or 3 ring heteroatoms. Particular examples of bicyclic heteroaryl groups containing a six membered ring fused to a five membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups. Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The terms “heterocyclyl,” as used herein, refer to a ring system containing one, two, or three rings, at least one of which containing one to four (e.g., one, two, or three) heteroatoms selected from the group consisting of nitrogen, oxygen, and sulfur, provided that the ring system does not contain aromatic rings that also include an endocyclic heteroatom. An unsubstituted heterocyclyl group contains a total of two to nine carbon atoms. The term heterocyclyl includes both monovalent species and divalent species. Examples of heterocyclyl groups are monocyclic and bicyclic groups containing from five to twelve ring members, and more usually from five to ten ring members. The heterocyclyl group can be, for example, a 5- or 6-membered monocyclic ring or a 9- or 10-membered bicyclic ring, for example, a bicyclic structure formed from fused five and six membered rings or two fused six membered rings. Each ring may contain up to about four heteroatoms typically selected from nitrogen, sulfur and oxygen. Non-limiting examples of heterocyclyl groups include, e.g., pyrrolidine, piperazine, piperidine, azepane, 1,4-diazepane, tetrahydrofuran, tetrahydropyran, oxepane, 1,4-dioxepane, tetrahydrothiophene, tetrahydrothiopyran, indoline, benzopyrrolidine, 2,3-dihydrobenzofuran, phthalan, isochroman, and 2,3-dihydrobenzothiophene.

The term “increase” or “restore” or “improve” may relate generally to the ability of one or more compounds of the invention to “increase” a relevant physiological or cellular response, which may be decreased in a disease state as described herein, and as measured according to routine techniques in the diagnostic art. Relevant physiological or cellular responses (in vivo or in vitro) will be apparent to persons skilled in the art, and may include, for example, improvements in the symptoms or pathology of a neuromuscular or neurologic disease. An “increase” in a response may be statistically significant as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% increase, including all integers in between.

The term “internucleoside linkage,” as used herein, represents a group or bond that forms a covalent linkage between adjacent nucleosides in an oligonucleotide. An internucleoside linkage is an unmodified internucleoside linkage or a modified internucleoside linkage. An “unmodified internucleoside linkage” is a phosphate (—O—P(O)(OH)—O—) internucleoside linkage (“phosphate phosphodiester”). A “modified internucleoside linkage” is an internucleoside linkage other than a phosphate phosphodiester. The two main classes of modified internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non-limiting examples of non-phosphorus internucleoside linkages include methylenemethylimino (—CH₂—N(CH₃)—O—), thiodiester (—O—C(O)—S—), thionocarbamate (—O—C(O)(NH)—S—), siloxane (—O—Si(H)₂—O—), and N,N′-dimethylhydrazine (—CH₂—N(CH₃)—N(CH₃)—). Phosphorothioate linkages are phosphodiester linkages and phosphotriester linkages in which one of the non-bridging oxygen atoms is replaced with a sulfur atom. In some embodiments, an internucleoside linkage is a group of the following structure:

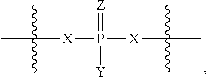

where
Z is O, S, or Se;
Y is —X-L-R¹;
each X is independently —O—, —S—, —N(-L-R¹)—, or L;
each L is independently a covalent bond or a linker (e.g., optionally substituted C₁₋₆₀ aliphatic linker or optionally substituted C₂₋₆₀ heteroaliphatic linker);
each R¹ is independently hydrogen, —S—S—R², —O—CO—R², —S—CO—R², optionally substituted C₁₋₉ heterocyclyl, or a hydrophobic moiety; and
each R² is independently optionally substituted C₁₋₁₀ alkyl, optionally substituted C₂₋₁₀ heteroalkyl, optionally substituted C₆₋₁₀ aryl, optionally substituted C₆₋₁₀ aryl C₁₋₆ alkyl, optionally substituted C₁₋₉ heterocyclyl, or optionally substituted C₁₋₉ heterocyclyl C₁₋₆ alkyl.

When L is a covalent bond, R¹ is hydrogen, Z is oxygen, and all X groups are —O—, the internucleoside group is known as a phosphate phosphodiester. When L is a covalent bond, R¹ is hydrogen, Z is sulfur, and all X groups are —O—, the internucleoside group is known as a phosphorothioate diester. When Z is oxygen, all X groups are —O—, and either (1) L is a linker or (2) R¹ is not a hydrogen, the internucleoside group is known as a phosphotriester. When Z is sulfur, all X groups are —O—, and either (1) L is a linker or (2) R¹ is not a hydrogen, the internucleoside group is known as a phosphorothioate triester. Non-limiting examples of phosphorothioate triester linkages and phosphotriester linkages are described in US 2017/0037399, the disclosure of which is incorporated herein by reference.

An “intron” refers to a nucleic acid region (within a gene) that is not translated into a protein. An intron is a non-coding section that is transcribed into a precursor mRNA (pre-mRNA), and subsequently removed by splicing during formation of the mature RNA.

The term “morpholino,” as used herein in reference to a class of oligonucleotides, represents an oligomer of at least 10 morpholino monomer units interconnected by morpholino internucleoside linkages. A morpholino includes a 5′ group and a 3′ group. For example, a morpholino may be of the following structure:

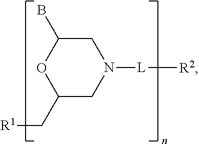

where
n is an integer of at least 10 (e.g., 12 to 30) indicating the number of morpholino subunits and associated groups L;
each B is independently a nucleobase;
R¹ is a 5′ group (R¹ may be referred to herein as a 5′ terminus);
R² is a 3′ group (R² may be referred to herein as a 3′ terminus); and
L is (i) a morpholino internucleoside linkage or, (ii) if L is attached to R², a covalent bond.

A 5′ group in morpholino may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, phosphorodiamidate, a bond to a peptide, a bond to a peptide/linker combination, an endosomal escape moiety, a neutral organic polymer, or a group of the following structure:

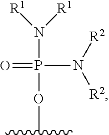

where
each R¹ is independently alkyl optionally substituted with ═O, —OH, and/or —NH₂; heteroalkyl optionally substituted with ═O, —OH, and/or —NH₂; cycloalkyl; heterocyclyl; heteroaryl; or -L-R³; where L is a linker that is alkyl, cycloalkyl, heteroalkyl, heterocyclyl, or heteroaryl, and R³ is a solid support, provided that no more than one R¹ is -L-R³; or two R¹ groups, together with the nitrogen atom to which they are attached, combine to form a heterocyclyl optionally substituted with alkyl optionally substituted with ═O, —OH, and/or —NH₂ or heteroalkyl optionally substituted with ═O, —OH, and/or —NH₂; and
each R² is independently alkyl optionally substituted with ═O, —OH, and/or —NH₂; heteroalkyl optionally substituted with ═O, —OH, and/or —NH₂; cycloalkyl; heterocyclyl; or heteroaryl.

Preferably, a 5′ group is a hydroxyl or a group of the following structure:

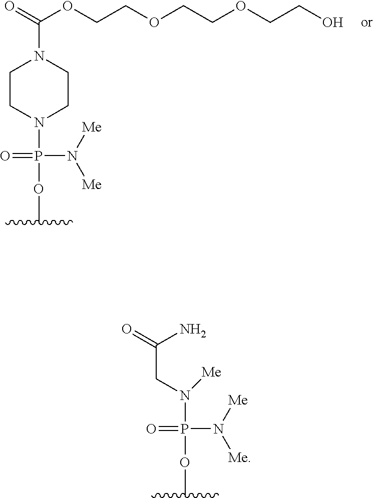 [SMILES: *OP(=O)(N(C)C)N1CCN(C(=O)OCCOCCOCCO)CC1 ; *OP(=O)(N(C)C)N(C)CC(N)=O]

A more preferred 5′ group is of the following structure:

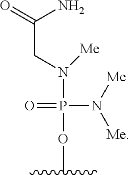 [SMILES: *OP(=O)(N(C)C)N(C)CC(N)=O]

A 3′ group in morpholino may be, e.g., hydrogen, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a bond to a peptide, a bond to a peptide/linker combination, a group for conjugation (e.g., a maleimide, an alkyl or aryl substituted with thiol, an alkynyl, or an alkyl substituted with azide), an endosomal escape moiety, or a neutral organic polymer.

In a conjugate of an oligonucleotide that is a morpholino and a peptide that is covalently bonded or linked to the oligonucleotide, the preferred 3′ group is a bond to a peptide or a bond to a peptide/linker combination.

The term “morpholino internucleoside linkage,” as used herein, represents a divalent group of the following structure:

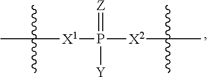

where
Z is O or S;
X¹ is a bond, —CH₂—, or —O—;
X² is a bond, —CH₂—O—, or —O—; and
Y is —NR₂, where each R is independently H or C₁₋₆ alkyl (e.g., methyl), or both R combine together with the nitrogen atom to which they are attached to form a C₂₋₉ heterocyclyl (e.g., N-piperazinyl); provided that one and only one of X¹ and X² is a bond. Preferably, the morpholino internucleoside linkage is —P(O)(Nme₂)O—. The morpholino internucleoside linkage is bonded through its phosphorus atom to the nitrogen atom of the morpholine ring in the morpholino subunit.

The term “morpholino subunit,” as used herein, refers to the following structure:

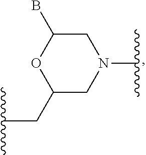

where B is a nucleobase.

The term “nucleobase,” as used herein, represents a nitrogen-containing heterocyclic ring found at the 1′ position of the ribofuranose/2′-deoxyribofuranose of a nucleoside. Nucleobases are unmodified or modified. As used herein, “unmodified” or “natural” nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and O-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e g., 5-substituted pyrimidines; 6-azapyrimidines; N2-, N6-, and/or O6-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example, 7-deazaadenine, 7-deazaguanine, 2-aminopyridine, or 2-pyridone. Further nucleobases include those disclosed in Merigan et al., U.S. Pat. No. 3,687,808, those disclosed in The Concise Encyclopedia of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

The term “nucleoside,” as used herein, represents sugar-nucleobase compounds and groups known in the art, as well as modified or unmodified 2′-deoxyribofuranrpose-nucleobase compounds and groups known in the art. The sugar may be ribofuranose. The sugar may be modified or unmodified. An unmodified ribofuranose-nucleobase is ribofuranose having an anomeric carbon bond to an unmodified nucleobase. Unmodified ribofuranose-nucleobases are adenosine, cytidine, guanosine, and uridine. Unmodified 2′-deoxyribofuranose-nucleobase compounds are 2′-deoxyadenosine, 2′-deoxycytidine, 2′-deoxyguanosine, and thymidine. The modified compounds and groups include one or more modifications selected from the group consisting of nucleobase modifications and sugar modifications described herein. A nucleobase modification is a replacement of an unmodified nucleobase with a modified nucleobase. A sugar modification may be, e.g., a 2′-substitution, locking, carbocyclization, or unlocking. A 2′-substitution is a replacement of 2′-hydroxyl in ribofuranose, e.g., with 2′-fluoro, 2′-methoxy, or 2′-(2-methoxy) ethoxy. Alternatively, a 2′-substitution may be a 2′-(ara) substitution, which corresponds to the following structure:

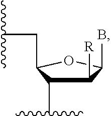

where B is a nucleobase, and R is a 2′-(ara) substituent (e.g., fluoro). 2′-(ara) substituents are known in the art and can be same as other 2′-substituents described herein. In some embodiments, 2′-(ara) substituent is a 2′-(ara)-F substituent (R is fluoro). A locking modification is an incorporation of a bridge between 4′-carbon atom and 2′-carbon atom of ribofuranose. Nucleosides having a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides. A “nucleoside” may also refer to a morpholino subunit.

The term “nucleotide,” as used herein, represents a nucleoside bonded to an internucleoside linkage or a monovalent group of the following structure —X¹—P(X²)(R¹)₂, where X¹ is O, S, or NH, and X² is absent, ═O, or ═S, and each R¹ is independently —OH, —N(R²)₂, or —O—CH₂CH₂CN, where each R² is independently an optionally substituted alkyl, or both R² groups, together with the nitrogen atom to which they are attached, combine to form an optionally substituted heterocyclyl.

The term “oligonucleotide,” as used herein, represents a structure containing 10 or more contiguous nucleosides covalently bound together by internucleoside linkages; a morpholino containing 10 or more morpholino subunits; or a peptide nucleic acid containing 10 or more peptide nucleic acid subunits. Preferably, an oligonucleotide is a morpholino. In some embodiments, the oligonucleotide is a phosphorothioate (PS) oligonucleotide. In some embodiments, the oligonucleotide is a 2′-O-alkyl oligoribonucleotide, e.g., a 2′-O-methyl oligoribonucleotide. In some embodiments, the oligonucleotide is a 2′-O-alkyl phosphorothioate, e.g., a 2′-O-methyl phosphorothioate.

The term “optionally substituted” refers to groups, structures, or molecules that may be substituted or unsubstituted as described for each respective group. The term “wherein a/any CH, CH₂, CH₃ group or heteroatom (i.e., NH) within a R¹ group is optionally substituted” means that (any) one of the hydrogen radicals of the R¹ group is substituted by a relevant stipulated group.

The term “pharmaceutically acceptable,” as used herein, refers to those compounds, materials, compositions, and/or dosage forms, which are suitable for contact with the tissues of an individual (e.g., a human), without excessive toxicity, irritation, allergic response and other problem complications commensurate with a reasonable benefit/risk ratio.

The term “pharmaceutically acceptable salt,” as used herein, means any pharmaceutically acceptable salt of a conjugate, oligonucleotide, or peptide disclosed herein. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “pharmaceutical composition,” as used herein, represents a composition containing an oligonucleotide described herein, formulated with a pharmaceutically acceptable excipient, and manufactured or sold with the approval of a governmental regulatory agency as part of a therapeutic regimen for the treatment of disease in a subject.

The term “reduce” or “inhibit” may relate generally to the ability of one or more compounds of the invention to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include, for example, reductions in the symptoms or pathology of a neuromuscular or neurologic disease. A “decrease” in a response may be statistically significant as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

“Steric blocking” refers to a therapeutic modality whereby a complementary antisense oligonucleotide binds to a target sequence present in an mRNA transcript and prevents the association of this transcript with other molecules, which may include proteins or RNA species. A steric blocking mechanism may be employed in the treatment of DM1 or DM2 or other genetic disorders and may result in the correction of downstream mis-splicing pathologies and their corresponding phenotypic expression, or may provide therapeutic benefit through other mechanisms of action.

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject. Non-limiting examples of diseases, disorders, and conditions include DMD, BMD, FSHD, DM1, DM2, CMT1a, CMT2a, FCD, FA and SMA.

A “sugar” or “sugar moiety,” includes naturally occurring sugars having a furanose ring or a structure that is capable of replacing the furanose ring of a nucleoside. Sugars included in the nucleosides of the invention may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the invention include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-S-2′-substituted ribose), bicyclic sugar moieties (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

“Treatment” and “treating,” as used herein, refer to the medical management of a subject with the intent to improve, ameliorate, or stabilize a disease, disorder, or condition (e.g., DMD, BMD, FSHD, DM1, DM2, CMT1a, FCD, FA or SMA). This term includes active treatment (treatment directed to improve DMD, BMD, FSHD, DM1, DM2, CMT1a, FCD, FA and SMA); palliative treatment (treatment designed for the relief of symptoms of DMD, BMD, FSHD, DM1, DM2, CMT1a, FCD, FA, and SMA); and supportive treatment (treatment employed to supplement another therapy).

Throughout the description and claims of this specification, the words “comprise” and “contain” and variations of them mean “including but not limited to,” and they are not intended to (and do not) exclude other moieties, additives, components, integers, or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references to “conjugates” also refer to salts and solvates thereof. As will be appreciated by one of skill in the art, the conjugates disclosed herein may include multiple ionizable and/or protonatable groups. Accordingly, a conjugate of the invention may be used in a salt form, such as acid addition salt, or in a substantially neutral form.

All references to “oligonucleotides” also refer to salts and solvates thereof.

Unless otherwise specified, all peptides are shown herein in N-terminus to C-terminus direction (left to right). Unless otherwise specified, all oligonucleotides are shown herein in 5′ to 3′ direction (left to right).

Features, integers, characteristics, compounds, chemical moieties, or groups described in conjunction with a particular aspect, embodiment or example of the invention are to be understood to be applicable to any other aspect, embodiment or example described herein unless incompatible therewith. All the features disclosed in this specification (including any accompanying claims, abstract and drawings), and/or all the steps of any method or process so disclosed, may be combined in any combination, except combinations where at least some of such features and/or steps are mutually exclusive.

### BRIEF DESCRIPTION OF THE DRAWINGS

FIG. 1 is a plot showing Dmd exon 23 skipping levels in quadriceps of wild type mice receiving PPMOs. The results are mean±SD, n=3-5.

FIG. 2 is a plot showing Dmd exon 23 skipping levels in the cardiac muscle of wild type mice receiving PPMOs. The results are mean±SD, n=3-5.

FIG. 3 is a scheme of PMO synthesis.

### DETAILED DESCRIPTION

In general, the invention provides new peptides that can advantageously be used for the delivery of therapeutic or diagnostic molecules, such as oligonucleotides. In some embodiments, the peptides are present within conjugates of the peptides and the therapeutic or diagnostic molecules (e.g., oligonucleotides), wherein a peptide is covalently bonded or covalently linked via a linker to a therapeutic or diagnostic molecule. As described further below, the conjugates can be used in the treatment of genetic diseases, e.g., neuromuscular and neurologic diseases, such as DMD, BMD, FSHD, DM1, DM2, CMT1a, CMT2a, FCD, FA, and SMA.

In some embodiments, the conjugate is of peptide and an oligonucleotide covalently bonded (i.e., directly) or linked via a non-cationic linker (e.g., via a linker including a total of one amino acid) or via an aliphatic dicarboxylic acid linker to the peptide, the peptide comprising a total of one hydrophobic domain and a total of one cationic domain, the cationic domain comprising at least 1 cationic amino acid residue, and the hydrophobic domain comprising at least 3 amino acid residues, provided that the peptide comprises a total of 5 to 40 (e.g., 6 to 40 or 7 to 40) amino acid residues and does not comprise any artificial amino acid residues.

In some embodiments, the conjugate is of a peptide and an oligonucleotide covalently bonded (i.e., directly) or linked via a non-cationic linker (e.g., via a linker including a total of one amino acid) or via an aliphatic dicarboxylic acid linker to the peptide, the peptide comprising a total of one hydrophobic domain and a total of one or two cationic domains flanking the hydrophobic domain, each cationic domain comprising at least one cationic amino acid residue, at least one of the cationic domains comprising a total of one cationic amino acid residue, and the hydrophobic domain comprising at least 3 amino acid residues, provided that the peptide comprises a total of 5 to 40 (e.g., 6 to 40 or 7 to 40) amino acid residues and does not comprise any artificial amino acid residues.

In some embodiments, the conjugate is of a peptide and an oligonucleotide covalently bonded (i.e., directly) or linked via a non-cationic linker (e.g., via a linker including a total of one amino acid) or via an aliphatic dicarboxylic acid linker to the peptide, the peptide comprising a total of one hydrophobic domain and a total of one or two cationic domains flanking the hydrophobic domain, each cationic domain comprising at least 1 cationic amino acid residue, all cationic domains collectively comprising a total of five or fewer cationic amino acid residue, and the hydrophobic domain comprising at least 3 amino acid residues, provided that the peptide comprises a total of 5 to 40 (e.g., 6 to 40 or 7 to 40) amino acid residues and does not comprise any artificial amino acid residues.

In some embodiments, the conjugate is of a peptide and an oligonucleotide covalently bonded or linked via a non-cationic linker (e.g., via a linker including a total of one amino acid) or via an aliphatic dicarboxylic acid linker to the peptide, the peptide comprising a total of one hydrophobic domain and a total of one or two cationic domains flanking the hydrophobic domain, each cationic domain comprising at least 1 cationic amino acid residue, at least one third of amino acid residues in the N-terminal cationic domain are histidines, and the hydrophobic domain comprising at least 3 amino acid residues, provided that the peptide comprises a total of 5 to 40 (e.g., 6 to 40 or 7 to 40) amino acid residues and does not comprise any artificial amino acid residues.

In some embodiments, the conjugate is of a peptide and an oligonucleotide covalently bonded (i.e., directly) or linked via a non-cationic linker (e.g., via a linker including a total of one amino acid) or via an aliphatic dicarboxylic acid linker to the peptide, the peptide being selected from the group consisting of: RBRRFQILYRBHBH (SEQ ID NO: 447), RBRFQILYRBHBH (SEQ ID NO: 448), RBFQILYRBHBH (SEQ ID NO: 449), RFQILYRBHBH (SEQ ID NO: 450), FQILYRBHBH (SEQ ID NO: 451), RBRRBRRFQILYBHBHB (SEQ ID NO: 452), RBRRBRRFQILYHBH (SEQ ID NO: 453), RBRRBRRFQILYBH (SEQ ID NO: 454), RBRRBRRFQILYH (SEQ ID NO: 455), RBRRBRRFQILY (SEQ ID NO: 456), BRRBRRFQILYRBHBH (SEQ ID NO: 457), RRBRRFQILYRBHBH (SEQ ID NO: 458), BRRFQILYRBHBH (SEQ ID NO: 459), RRFQILYRBHBH (SEQ ID NO: 460), RBRRBRRFQILYRBHB (SEQ ID NO: 461), RBRRBRRFQILYRBH (SEQ ID NO: 462), RBRRBRRFQILYRB (SEQ ID NO: 463), RBRRBRRFQILYR (SEQ ID NO: 464), HRBHRBHRBFQILYHRBRH (SEQ ID NO: 465), HRBHRBHRBFQILYHRBHRBHR (SEQ ID NO: 466), HRBHRBFQILYHRBHR (SEQ ID NO: 467), HRBFQILYHR (SEQ ID NO: 468), RBRBRBFQILYRBRBR (SEQ ID NO: 469), RBRBFQILYRBR (SEQ ID NO: 470), RBFQILYR (SEQ ID NO: 471), HRHRHRFQILYHRHRHR (SEQ ID NO: 472), HRHRFQILYHRHR (SEQ ID NO: 473), HRFQILYHR (SEQ ID NO: 474), RRRRRRFQILY (SEQ ID NO: 475), FQILYRRRRRR (SEQ ID NO: 476), RRRRRRFQILYRRRRRR (SEQ ID NO: 477), RBRRBRFQILYBR (SEQ ID NO: 478), and RBRRBRFQILY (SEQ ID NO: 479).

In some embodiments, the peptide of the conjugate is (RBRRBRFQILYBR (SEQ ID NO: 478)). In some embodiments, the peptide of the conjugate is (RBRRBRFQILY (SEQ ID NO: 479)). In some embodiments, the peptide of the conjugate is (RBRRBRRFQILY (SEQ ID NO: 456)). In some embodiments, the peptide of the conjugate is (RRFQILYRBHBH (SEQ ID NO: 460)). In some embodiments, the peptide of the conjugate is rBrrBrfqilyBrBr (SEQ ID NO: 510), where lower case letters indicate D-amino acids.

In some embodiments, the conjugate is of a peptide and an oligonucleotide covalently bonded (i.e., directly) or linked via a non-cationic linker (e.g., via a linker including a total of one amino acid) or via an aliphatic dicarboxylic acid linker to the peptide, the peptide being selected from the group consisting of: RBRRFQILYRBHBHB (SEQ ID NO: 481), RBRFQILYRBHBHB (SEQ ID NO: 482), RBFQILYRBHBHB (SEQ ID NO: 483), RFQILYRBHBHB (SEQ ID NO: 484), FQILYRBHBHB, RBRRBRRFQILYBHBHB (SEQ ID NO: 452), RBRRBRRFQILYHBHB (SEQ ID NO: 486), RBRRBRRFQILYBHB (SEQ ID NO: 487), RBRRBRRFQILYHB (SEQ ID NO: 488), RBRRBRRFQILYB (SEQ ID NO: 489), RBRRBRRFQILYE (SEQ ID NO: 416), RBRRBRRFQILY (SEQ ID NO: 456), BRRBRRFQILYRBHBHB (SEQ ID NO: 490), RRBRRFQILYRBHBHB (SEQ ID NO: 491), BRRFQILYRBHBHB (SEQ ID NO: 492), RRFQILYRBHBHB (SEQ ID NO: 493), RRFQILYRBHBHE (SEQ ID NO: 417), RBRRBRRFQILYRBHBB (SEQ ID NO: 494), RBRRBRRFQILYRBHB (SEQ ID NO: 461), RBRRBRRFQILYRBB (SEQ ID NO: 495), RBRRBRRFQILYRB (SEQ ID NO: 463), HRBHRBHRBFQILYHRBRHB (SEQ ID NO: 496), HRBHRBHRBFQILYHRBHRBHRB (SEQ ID NO: 497), HRBHRBFQILYHRBHRB (SEQ ID NO: 498), HRBFQILYHRB (SEQ ID NO: 499), RBRBRBFQILYRBRBRB (SEQ ID NO: 500), RBRBFQILYRBRB (SEQ ID NO: 501), RBFQILYRB (SEQ ID NO: 502), HRHRHRFQILYHRHRHRB (SEQ ID NO: 503), HRHRFQILYHRHRB (SEQ ID NO: 504), HRFQILYHRB (SEQ ID NO: 505), RRRRRRFQILYB (SEQ ID NO: 506), FQILYRRRRRRB (SEQ ID NO: 507), RRRRRRFQILYRRRRRRB (SEQ ID NO: 508), RBRRBRFQILYBRE (SEQ ID NO: 415), and RBRRBRFQILYE (SEQ ID NO: 509). In another aspect, the conjugate comprises a peptide as set forth previously in this paragraph, but with the C-terminal amino acid removed or removed and replaced with a linker, e.g., as described herein (e.g., a B or an E linker).

In some embodiments, the peptide of the conjugate is Dpep1.9b-del2 (RBRRBRFQILYBRE) (SEQ ID NO: 415). In some embodiments, the peptide of the conjugate is Dpep1.9b-del4 (RBRRBRFQILYE) (SEQ ID NO: 509). In some embodiments, the peptide of the conjugate is peptide E5-E (RBRRBRRFQILYE) (SEQ ID NO: 416). In some embodiments, the peptide of the conjugate is peptide G5-E (RRFQILYRBHBHE) (SEQ ID NO: 417). In some embodiments, the peptide of the conjugate is rBrrBrfqilyBrBre (SEQ ID NO: 405), where lower case letters indicate D-amino acids.

The peptides can include at least one positively charged domain and at least one hydrophobic domain. Without wishing to be bound by theory, the peptide may act as a cell-penetrating peptide to enhance the activity of the conjugated oligonucleotide, e.g., by improving intracellular delivery of the conjugated oligonucleotide. Advantageously, as described in the Examples below, the conjugates disclosed herein exhibit reduced toxicity relative to certain alternative peptide structures.

In the case of therapeutic molecules that are oligonucleotides, the oligonucleotide may be, for example, an antisense oligonucleotide that is complementary to a target sequence within or proximal to any one of exons 8-55 (e.g., exon 8, exon 23, exon 44, exon 45, exon 50, exon 51, exon 52, exon 53 or exon 55) of a human dystrophin gene. Alternatively, the oligonucleotide may be complementary to a target sequence within or proximal to intron 7 of a transcript of a human SMN2 gene. Alternatively, the oligonucleotide may be complementary to a CUG repeat sequence (e.g., at least 9 contiguous nucleobases are complementary to the CUG repeat sequence). Alternatively, the oligonucleotide may be complimentary to a CCUG repeat sequence.

In some embodiments, the antisense oligonucleotide sequence is for inducing exon skipping of a single exon of the dystrophin gene for use in the treatment of DMD (e.g., those that have a target sequence in the dystrophin transcript). In some embodiments, the single exon is selected from any exon implicated in DMD, which may be any exon in the dystrophin gene (e.g., a human dystrophin gene), such as for example, exon 8, 23, 44, 45, 50, 51, 52, 53, or 55. In some embodiments, the antisense oligonucleotide sequence is for promoting exon 7 inclusion on splicing of SMN2 pre-mRNA and thus may be for use in the treatment of spinal muscular atrophy (SMA). In some embodiments, the antisense oligonucleotide sequence binds to SMN2 pre-mRNA intron 7 to promote exon 7 inclusion.

In some embodiments, the antisense oligonucleotide sequence is for reducing adverse effects related to the presence of a CTG repeat expansion in the DMPK gene. By targeting r(CUG)^{exp} (e.g., the oligonucleotide may have at least 9 contiguous nucleobases complementary to a CUG repeat sequence) in the 3′-untranslated region of the DMPK transcript, the oligonucleotide can ameliorate the pathologies associated with this gain-of-function repeat expansion in DM1.

In some embodiments, the antisense oligonucleotide sequence is for reducing adverse effects related to the presence of a CCTG repeat expansion in the CNBP gene. By targeting r(CCUG)^{exp} (e.g., the oligonucleotide may have at least 8 contiguous nucleobases complementary to a CCUG repeat sequence) in the first intron region of CNBP transcripts, the oligonucleotide can ameliorate the pathologies associated with this gain-of-function repeat expansion in DM2.

In some embodiments, the oligonucleotide of the conjugate is an oligonucleotide complementary to the pre-mRNA of a gene target. Accordingly, when reference is made herein to targeting a gene, this includes targeting of an RNA transcript of the gene (e.g., a pre-mRNA transcript).

In some embodiments, the oligonucleotide complementary to the pre-mRNA of a gene target gives rise to a steric blocking event that alters the pre-mRNA leading to an altered mRNA and hence a protein of altered sequence. In some embodiments, the gene target is the dystrophin gene. In some embodiments, the steric blocking event may be exon inclusion or exon skipping. In some embodiments, the steric blocking event is exon skipping, e.g., exon skipping of a single exon of the dystrophin gene. Optionally, lysine residues may be added to one or both ends of an oligonucleotide (such as a PMO or PNA) before attachment to the peptide to improve water solubility.

In some embodiments, the oligonucleotide has a molecular weight of less than 15,000 Da, e.g., less than 10,000 Da, e.g., less than 5,000 Da, or e.g., less than 3,000 Da.

In some embodiments, the peptide is covalently linked to the oligonucleotide at the C-terminus.

In some embodiments, the peptide is covalently linked to the oligonucleotide through a linker. The linker may act as a spacer to separate the peptide sequence from the oligonucleotide.

The linker may be selected from any suitable sequence.

In some embodiments, the linker is present between the peptide and the oligonucleotide. In some embodiments, the linker is a separate group to the peptide and the oligonucleotide. Accordingly, the linker may comprise artificial amino acids.

In some embodiments, the conjugate comprises the peptide covalently linked via a linker to an oligonucleotide. In some embodiments, the conjugate comprises the following structure:
[peptide]-[linker]-[oligonucleotide]

In some embodiments, the conjugate consists of the following structure:
[peptide]-[linker]-[oligonucleotide]

In some embodiments, any of the peptides listed herein may be used in the conjugate according to the invention.

Preferably, the oligonucleotide is a morpholino (more preferably, a morpholino with all morpholino internucleoside linkages being —P(O)(Nme₂)O—).

In some embodiments, the oligonucleotide is a phosphorothioate, e.g., as described herein. In some embodiments, the oligonucleotide is a 2′-O-alkyl oligonucleotide, e.g., 2′-O-methyl oligonucleotide and/or 2′-O-alkyl phosphorothioate, such as 2′-O-methyl phosphorothioate.

In some embodiments, the oligonucleotide is a PNA.

Additional information concerning the peptides, conjugates containing the peptides, linkers, molecules to which the peptides may be linked to form the conjugates, pharmaceutical compositions, and methods is provided as follows.

### Peptides

The present invention relates to short cell-penetrating peptides for use in transporting therapeutic cargo molecules in the treatment of medical conditions.

The peptide has a sequence that is a contiguous single molecule, therefore the domains of the peptide are contiguous. In some embodiments, the peptide comprises several domains in a linear arrangement between the N-terminus and the C-terminus. In some embodiments, the domains are selected from cationic domains and hydrophobic domains described above. In some embodiments, the peptide consists of cationic domains and hydrophobic domains wherein the domains are as defined herein.

Each domain has common sequence characteristics as described herein, but the exact sequence of each domain is capable of variation and modification. Thus, a range of sequences is possible for each domain. The combination of each possible domain sequence yields a range of peptide structures, each of which form part of the present invention. Features of the peptide structures are described herein.

In some embodiments, the peptide includes a total of one hydrophobic domain and a total of one cationic domain, the cationic domain comprising at least 1 cationic amino acid residue, and the hydrophobic domain comprising at least 3 amino acid residues, provided that the peptide comprises a total of 5 to 40 (e.g., 6 to 40 or 7 to 40) amino acid residues and does not comprise any artificial amino acid residues.

In some embodiments, the peptide includes a total of one hydrophobic domain and a total of one or two cationic domains flanking the hydrophobic domain, each cationic domain comprising at least one cationic amino acid residue, at least one of the cationic domains comprising a total of one cationic amino acid residue, and the hydrophobic domain comprising at least 3 amino acid residues, provided that the peptide comprises a total of 5 to 40 (e.g., 6 to 40 or 7 to 40) amino acid residues and does not comprise any artificial amino acid residues.

In some embodiments, the peptide includes a total of one hydrophobic domain and a total of one or two cationic domains flanking the hydrophobic domain, each cationic domain comprising at least 1 cationic amino acid residue, all cationic domains collectively comprising a total of five or fewer cationic amino acid residue, and the hydrophobic domain comprising at least 3 amino acid residues, provided that the peptide comprises a total of 5 to 40 (e.g., 6 to 40 or 7 to 40) amino acid residues and does not comprise any artificial amino acid residues.

In some embodiments, the peptide includes a total of one hydrophobic domain and a total of one or two cationic domains flanking the hydrophobic domain, each cationic domain comprising at least 1 cationic amino acid residue, at least one third of amino acid residues in the N-terminal cationic domain are histidines, and the hydrophobic domain comprising at least 3 amino acid residues, provided that the peptide comprises a total of 5 to 40 (e.g., 6 to 40 or 7 to 40) amino acid residues and does not comprise any artificial amino acid residues.

In some embodiments, the peptide is selected from the group consisting of: RBRRFQILYRBHBH (SEQ ID NO: 447), RBRFQILYRBHBH (SEQ ID NO: 448), RBFQILYRBHBH (SEQ ID NO: 449), RFQILYRBHBH (SEQ ID NO: 450), FQILYRBHBH (SEQ ID NO: 451), RBRRBRRFQILYBHBHB (SEQ ID NO: 452), RBRRBRRFQILYHBH (SEQ ID NO: 453), RBRRBRRFQILYBH (SEQ ID NO: 454), RBRRBRRFQILYH (SEQ ID NO: 455), RBRRBRRFQILY (SEQ ID NO: 456), BRRBRRFQILYRBHBH (SEQ ID NO: 457), RRBRRFQILYRBHBH (SEQ ID NO: 458), BRRFQILYRBHBH (SEQ ID NO: 459), RRFQILYRBHBH (SEQ ID NO: 460), RBRRBRRFQILYRBHB (SEQ ID NO: 461), RBRRBRRFQILYRBH (SEQ ID NO: 462), RBRRBRRFQILYRB (SEQ ID NO: 463), RBRRBRRFQILYR (SEQ ID NO: 464), HRBHRBHRBFQILYHRBRH (SEQ ID NO: 465), HRBHRBHRBFQILYHRBHRBHR (SEQ ID NO: 466), HRBHRBFQILYHRBHR (SEQ ID NO: 467), HRBFQILYHR (SEQ ID NO: 468), RBRBRBFQILYRBRBR (SEQ ID NO: 469), RBRBFQILYRBR (SEQ ID NO: 470), RBFQILYR (SEQ ID NO: 471), HRHRHRFQILYHRHRHR (SEQ ID NO: 472), HRHRFQILYHRHR (SEQ ID NO: 473), HRFQILYHR (SEQ ID NO: 474), RRRRRRFQILY (SEQ ID NO: 475), FQILYRRRRRR (SEQ ID NO: 476), RRRRRRFQILYRRRRRR (SEQ ID NO: 477), RBRRBRFQILYBR (SEQ ID NO: 478), and RBRRBRFQILY (SEQ ID NO: 479). In some embodiments, the peptide is rBrrBrfqilyBrBr (SEQ ID NO: 510), where lower case letters indicate D-amino acids.

In some embodiments, the peptide is selected from the group consisting of: RBRRFQILYRBHBHB (SEQ ID NO: 481), RBRFQILYRBHBHB (SEQ ID NO: 482), RBFQILYRBHBHB (SEQ ID NO: 483), RFQILYRBHBHB (SEQ ID NO: 484), FQILYRBHBHB (SEQ ID NO: 485), RBRRBRRFQILYBHBHB (SEQ ID NO: 452), RBRRBRRFQILYHBHB (SEQ ID NO: 486), RBRRBRRFQILYBHB (SEQ ID NO: 487), RBRRBRRFQILYHB (SEQ ID NO: 488), RBRRBRRFQILYB (SEQ ID NO: 489), RBRRBRRFQILYE (SEQ ID NO: 416), RBRRBRRFQILY (SEQ ID NO: 456), BRRBRRFQILYRBHBHB (SEQ ID NO: 490), RRBRRFQILYRBHBHB (SEQ ID NO: 491), BRRFQILYRBHBHB (SEQ ID NO: 492), RRFQILYRBHBHB (SEQ ID NO: 493), RRFQILYRBHBHE (SEQ ID NO: 417), RBRRBRRFQILYRBHBB (SEQ ID NO: 494), RBRRBRRFQILYRBHB (SEQ ID NO: 461), RBRRBRRFQILYRBB (SEQ ID NO: 495), RBRRBRRFQILYRB (SEQ ID NO: 463), HRBHRBHRBFQILYHRBRHB (SEQ ID NO: 496), HRBHRBHRBFQILYHRBHRBHRB (SEQ ID NO: 497), HRBHRBFQILYHRBHRB (SEQ ID NO: 498), HRBFQILYHRB (SEQ ID NO: 499), RBRBRBFQILYRBRBRB (SEQ ID NO: 500), RBRBFQILYRBRB (SEQ ID NO: 501), RBFQILYRB (SEQ ID NO: 502), HRHRHRFQILYHRHRHRB (SEQ ID NO: 503), HRHRFQILYHRHRB (SEQ ID NO: 504), HRFQILYHRB (SEQ ID NO: 505), RRRRRRFQILYB (SEQ ID NO: 506), FQILYRRRRRRB (SEQ ID NO: 507), RRRRRRFQILYRRRRRRB (SEQ ID NO: 508), RBRRBRFQILYBRE (SEQ ID NO: 415), and RBRRBRFQILYE (SEQ ID NO: 509). In some embodiments, the peptide of the conjugate is Dpep1.9b-del2 (RBRRBRFQILYBRE) (SEQ ID NO: 415). In some embodiments, the peptide of the conjugate is Dpep1.9b-del4 (RBRRBRFQILYE) (SEQ ID NO: 509). In some embodiments, the peptide of the conjugate is peptide E5-E (RBRRBRRFQILYE) (SEQ ID NO: 416). In some embodiments, the peptide of the conjugate is peptide G5-E (RRFQILYRBHBHE) (SEQ ID NO: 417). In some embodiments, the peptide of the conjugate is rBrrBrfqilyBrBre (SEQ ID NO: 405), where lower case letters indicate D-amino acids. In another aspect, the peptide is as set forth previously in this paragraph, but with the C-terminal amino acid removed or removed and replaced with a linker, e.g., as described herein (e.g., a B or an E linker).

In some embodiments, the peptide does not contain aminohexanoic acid residues. In some embodiments, the peptide does not contain any form of aminohexanoic acid residues. In some embodiments, the peptide does not contain 6-aminohexanoic acid residues.

In some embodiments, the peptide contains only natural amino acid residues and therefore consists of natural amino acid residues.

In some embodiments, artificial amino acids such as 6-aminohexanoic acid that are typically used in cell-penetrating peptides are replaced by natural amino acids. In some embodiments, the artificial amino acids such as 6-aminohexanoic acid that are typically used in cell-penetrating peptides are replaced by amino acids selected from beta-alanine, serine, proline, arginine, and histidine or hydroxyproline.

In some embodiments, aminohexanoic acid is replaced by beta-alanine. In some embodiments, 6-aminohexanoic acid is replaced by beta-alanine

In some embodiments, aminohexanoic acid is replaced by histidine. In some embodiments, 6-aminohexanoic acid is replaced by histidine.

In some embodiments, aminohexanoic acid is replaced by hydroxyproline. In some embodiments, 6-aminohexanoic acid is replaced by hydroxyproline.

In some embodiments, the artificial amino acids such as 6-aminohexanoic acid that are typically used in cell-penetrating peptides may be replaced by a combination of any of beta-alanine, serine, proline, arginine, and histidine or hydroxyproline, e.g., a combination of any of beta-alanine, histidine, and hydroxyproline.

In some embodiments, there is provided a peptide having a total length of 40 amino acid residues or less, the peptide comprising: two or more cationic domains each comprising at least 4 amino acid residues; and one or more hydrophobic domains each comprising at least 3 amino acid residues; wherein at least one cationic domain comprises histidine residues. In some embodiments, wherein at least one cationic domain is histidine rich.

In some embodiments, what is meant by histidine rich is defined herein in relation to the cationic domains.

In some embodiments, the peptides comprise L-amino acids, e.g., all chiral amino acids are L-amino acids. In some embodiments, the peptides comprise D-amino acids, e.g., all chiral amino acids are D-amino acids. In some embodiments, the peptides comprise L- and D-amino acids.

In some embodiments, the peptides comprise at least 1, 2, 3, 4, 5, 6, 7, 8, 9, 10, 11, 12, 13, 14, 16, 17, 18, 19, 20, 21, 22, 23, 24, or 25 D-amino acid(s). In some embodiments, in a peptide including D-amino acids as noted above, the remainder of the amino acids in such a peptide, if any, are L-amino acids or achiral amino acids.

In some embodiments, further groups may be present such as a linker, terminal modification and/or oligonucleotide.

In some embodiments, the peptide is N-terminally modified.

In some embodiments, the peptide is N-acetylated, N-methylated, N-trifluoroacetylated, N-trifluoromethylsulfonylated, or N-methylsulfonylated. In some embodiments, the peptide is N-acetylated.

Optionally, the N-terminus of the peptide may be unmodified.

In some embodiments, the peptide is C-terminal modified.

In some embodiments, the peptide comprises a C-terminal modification selected from: carboxy-, thioacid-, aminooxy-, hydrazino-, thioester-, azide, strained alkyne, strained alkene, aldehyde-, thiol or haloacetyl-group.

Advantageously, the C-terminal modification provides a means for linkage of the peptide to the oligonucleotide.

Accordingly, the C-terminal modification may comprise the linker and vice versa. In some embodiments, the C-terminal modification may consist of the linker or vice versa. Suitable linkers are described herein.

In some embodiments, the peptide comprises a C-terminal carboxyl group.

In some embodiments, the C-terminal carboxyl group is provided by a glycine or beta-alanine residue.

In some embodiments, the C terminal carboxyl group is provided by a beta-alanine residue. In some embodiments, the C terminal beta-alanine residue is a linker.

The peptide of the present invention may have a total length of 40 amino acid residues or less. The peptide may therefore be regarded as an oligopeptide.

In some embodiments, the peptide has a total length of 3-30 amino acid residues, e.g., of 5-25 amino acid residues, of 10-25 amino acid residues, of 13-23 amino acid residues, of 15-20 amino acid residues.

In some embodiments, the peptide has a total length of at least 12, at least 13, at least 14, at least 15, at least 16, at least 17 amino acid residues.

In some embodiments, the peptide is capable of penetrating cells. The peptide may therefore be regarded as a cell-penetrating peptide.

In some embodiments, the peptide is for attachment to an oligonucleotide. In some embodiments, the peptide is for transporting an oligonucleotide into a target cell. In some embodiments, the peptide is for delivering an oligonucleotide into a target cell. The peptide may therefore be regarded as a carrier peptide.

In some embodiments, the peptide is capable of penetrating into cells and tissues, e.g., into the nucleus of cells. In some embodiments, into muscle tissues.

### Cationic Domain

In the peptides described herein, each cationic domain includes at least 1 cationic amino acid residue. In some embodiments, each cationic domain includes at least one cationic amino acid residue, and at least one of the cationic domains includes a total of one cationic amino acid residue. In some embodiments, all cationic domains collectively include a total of seven or fewer (e.g., five or fewer) cationic amino acid residues.

In some embodiments, the peptide includes a total of one or two cationic domains flanking the hydrophobic domain, each cationic domain comprising at least 1 cationic amino acid residue.

In some embodiments, the peptide comprises up to 4 cationic domains, e.g., up to 3 cationic domains.

In some embodiments, the peptide comprises 2 cationic domains, e.g., a total of two cationic domains.

In some embodiments, the peptide comprises two or more cationic domains each having a length of at least 4 amino acid residues.

In some embodiments, each cationic domain has a length of between 4 to 12 amino acid residues, e.g., a length of between 4 to 7 amino acid residues.

In some embodiments, each cationic domain has a length of 4, 5, 6, or 7 amino acid residues.

In some embodiments, each cationic domain is of similar length, e.g., each cationic domain is the same length.

In some embodiments, each cationic domain comprises cationic amino acids and may also contain polar and or nonpolar amino acids.

Non-polar amino acids may be selected from: alanine, beta-alanine, proline, glycine, cysteine, valine, leucine, isoleucine, methionine, tryptophan, and phenylalanine.

Polar amino acids may be selected from: serine, asparagine, hydroxyproline, histidine, arginine, threonine, tyrosine, and glutamine. In some embodiments, the selected polar amino acids do not have a negative charge.

Cationic amino acids may be selected from: arginine, histidine, and lysine.

In some embodiments, each cationic domain does not comprise anionic or negatively charged amino acid residues. In some embodiments, each cationic domain comprises arginine, histidine, beta-alanine, hydroxyproline and/or serine residues.

In some embodiments, each cationic domain consists of arginine, histidine, beta-alanine, hydroxyproline and/or serine residues.

In some embodiments, each cationic domain comprises at least 40%, at least 45%, or at least 50% cationic amino acids.

In some embodiments, each cationic domain comprises a majority of cationic amino acids. In some embodiments, each cationic domain comprises at least 55%, at least 60%, at least 65% at least 70%, at least 75%, at least 80%, at least 85%, at least 90%, or at least 95% cationic amino acids.

In some embodiments, each cationic domain comprises an isoelectric point (pI) of at least 7.5, at least 8.0, at least 8.5, at least 9.0, at least 9.5, at least 10.0, at least 10.5, at least 11.0, at least 11.5, at least 12.0.

In some embodiments, each cationic domain comprises an isoelectric point (pI) of at least 10.0.

In some embodiments, each cationic domain comprises an isoelectric point (pI) of between 10.0 and 13.0

In some embodiments, each cationic domain comprises an isoelectric point (pI) of between 10.4 and 12.5.

In some embodiments, the isoelectric point of a cationic domain is calculated at physiological pH by any suitable means available in the art. In some embodiments, by using the I PC (www.isoelectric.org) a web-based algorithm developed by Lukasz Kozlowski, Biol Direct. 2016; 11:55. DOI: 10.1186/s 13062-016-0159-9.

In some embodiments, each cationic domain comprises at least 1 cationic amino acid, e.g., 1-5 cationic amino acids. In some embodiments, each cationic domain comprises at least 2 cationic amino acids, e.g., 2-5 cationic amino acids.

In some embodiments, each cationic domain is arginine rich and/or histidine rich. In some embodiments, a cationic domain may contain both histidine and arginine.

In some embodiments, each cationic domain comprises a majority of arginine and/or histidine residues.

In some embodiments, each cationic domain comprises at least 40%, at least 45%, at least 50%, at least 55%, at least 60%, at least 60%, at least 65%, least 70% arginine and/or histidine residues. In some embodiments, a cationic domain may comprise at least 40%, at least 45%, at least 50%, at least 55%, at least 60%, at least 60%, at least 65%, least 70% arginine residues.

In some embodiments, a cationic domain may comprise at least 40%, at least 45%, at least 50%, at least 55%, at least 60%, at least 60%, at least 65%, least 70% histidine residues.

In some embodiments, a cationic domain may comprise a total of between 1-5 histidine and 1-5 arginine residues. In some embodiments, a cationic domain may comprise between 1-5 arginine residues. In some embodiments, a cationic domain may comprise between 1-5 histidine residues. In some embodiments, a cationic domain may comprise a total of between 2-5 histidine and 3-5 arginine residues. In some embodiments, a cationic domain may comprise between 3-5 arginine residues. In some embodiments, a cationic domain may comprise between 2-5 histidine residues.

In some embodiments, each cationic domain does not comprise any beta-alanine residues. In some embodiments, each cationic domain comprises one or more beta-alanine residues. In some embodiments, each cationic domain may comprise a total of between 2-5 beta-alanine residues, e.g., a total of 2 or 3 beta-alanine residues.

In some embodiments, a cationic domain may comprise one or more hydroxyproline residues or serine residues.

In some embodiments, a cationic domain may comprise between 1-2 hydroxyproline residues. In some embodiments, a cationic domain may comprise between 1-2 serine residues.

In some embodiments, all of the cationic amino acids in a given cationic domain may be histidine, alternatively, e.g., all of the cationic amino acids in a given cationic domain may be arginine.

In some embodiments, the peptide may comprise at least one histidine rich cationic domain. In some embodiments, the peptide may comprise at least one arginine rich cationic domain.

In some embodiments, the peptide may comprise at least one arginine rich cationic domain and at least one histidine rich cationic domain.

In some embodiments, the peptide comprises two arginine rich cationic domains.

In some embodiments, the peptide comprises two histidine rich cationic domains.

In some embodiments, the peptide comprises two arginine and histidine rich cationic domains.

In some embodiments, the peptide comprises one arginine rich cationic domain and one histidine rich cationic domain. In some embodiments, each cationic domain comprises no more than 3 contiguous arginine residues, e.g., no more than 2 contiguous arginine residues.

In some embodiments, each cationic domain comprises no contiguous histidine residues.

In some embodiments, each cationic domain comprises arginine, histidine and/or beta-alanine residues. In some embodiments, each cationic domain comprises a majority of arginine, histidine and/or beta-alanine residues. In some embodiments, at least 70%, at least 75%, at least 80%, at least 85%, at least 90%, at least 95%, 100% of the amino acid residues in each cationic domain are arginine, histidine and/or beta-alanine residues. In some embodiments, each cationic domain consists of arginine, histidine and/or beta-alanine residues.

In some embodiments, the peptide comprises a first cationic domain comprising arginine and beta-alanine residues and a second cationic domain comprising arginine and beta-alanine residues.

In some embodiments, the peptide comprises a first cationic domain comprising arginine and beta-alanine resides, and a second cationic domain comprising histidine, beta-alanine, and optionally arginine residues.

In some embodiments, the peptide comprises a first cationic domain comprising arginine and beta-alanine resides, and a second cationic domain comprising histidine and beta-alanine residues.

In some embodiments, the peptide comprises a first cationic domain consisting of arginine and beta-alanine residues and a second cationic domain consisting of arginine and beta-alanine residues.

In some embodiments, the peptide comprises a first cationic domain consisting of arginine and beta-alanine residues and a second cationic domain consisting of arginine, histidine and beta-alanine residues.

In some embodiments, the peptide comprises at least two cationic domains, e.g., these cationic domains form the arms of the peptide. In some embodiments, the cationic domains are located at the N and C termini of the peptide. In some embodiments, therefore, the cationic domains may be known as the cationic arm domains.

In some embodiments, the peptide comprises two cationic domains, wherein one is located at the N-terminus of the peptide and one is located at the C-terminus of the peptide. In some embodiments, no further amino acids or domains are present at the N-terminus and C-terminus of the peptide, with the exception of other groups such as a terminal modification, linker and/or oligonucleotide. For the avoidance of doubt, such other groups may be present in addition to ‘the peptide’ described and claimed herein. In some embodiments, therefore each cationic domain forms the terminus of the peptide. In some embodiments, this does not preclude the presence of a further linker group as described herein.

In some embodiments, the peptide may comprise up to 4 cationic domains. In some embodiments, the peptide comprises two cationic domains.

In some embodiments, the peptide comprises two cationic domains that are both arginine rich.

In some embodiments, the peptide comprises one cationic domain that is arginine rich.

In some embodiments, the peptide comprises two cationic domains that are both arginine and histidine rich.

In some embodiments, the peptide comprises one cationic domain that is arginine rich and one cationic domain that is histidine rich.

In some embodiments, at least one cationic domain (e.g., all cationic domains) is selected from the group consisting of RBRR (SEQ ID NO: 419), RBR, RB, R, RBRRBRR (SEQ ID NO: 420), RRBRR (SEQ ID NO: 425), BRR, RR, HRBHRBHRB (SEQ ID NO: 422), HRBHRB (SEQ ID NO: 423), HRB, RBRBRB (SEQ ID NO: 424), RBRB (SEQ ID NO: 425), RB, HRHRHR (SEQ ID NO: 426), HRHR (SEQ ID NO: 427), HR, RRRRRR (SEQ ID NO: 428), RBRRBR (SEQ ID NO: 429), RBHBHB (SEQ ID NO: 430), RBHBH (SEQ ID NO: 431), BHBHB (SEQ ID NO: 432), BHBH (SEQ ID NO: 433), HBHB (SEQ ID NO: 434), HBH, BHB, BH, HB, H, RBHBHE (SEQ ID NO: 435), RBHBB (SEQ ID NO: 436), RBHB (SEQ ID NO: 437), RBB, HRBRHB (SEQ ID NO: 438), HRBHRBHR (SEQ ID NO: 439), HRBHR (SEQ ID NO: 440), HRB, RBRBR (SEQ ID NO: 441), HRHRHRB (SEQ ID NO: 442), HRHRB (SEQ ID NO: 443), HRBRH (SEQ ID NO: 444), HRB, RRRRRRB (SEQ ID NO: 445), BRE, and BR, e.g., RBRR (SEQ ID NO: 419), RBR, RB, R, RBRRBRR (SEQ ID NO: 420), RRBRR (SEQ ID NO: 421), BRR, RR, HRBHRBHRB (SEQ ID NO: 422), HRBHRB (SEQ ID NO: 423), HRB, RBRBRB (SEQ ID NO: 424), RBRB (SEQ ID NO: 425), RB, HRHRHR (SEQ ID NO: 426), HRHR (SEQ ID NO: 427), HR, RRRRRR (SEQ ID NO: 428), RBRRBR (SEQ ID NO: 429), RBHBH (SEQ ID NO: 431), BHBH (SEQ ID NO: 433), HBH, BH, H, RBHB (SEQ ID NO: 437), HRBRH (SEQ ID NO: 444), HRBHRBHR (SEQ ID NO: 439), HRBHR (SEQ ID NO: 440), RBRBR (SEQ ID NO: 441), and BR. In some embodiments, at least one cationic domain (e.g., all cationic domains) is selected from the group consisting of RBR, RBRB (SEQ ID NO: 425), RB, R, RBRRBRR (SEQ ID NO: 420), BRR, BR, RR, HRBHRBHRB (SEQ ID NO: 422), HRBHRB (SEQ ID NO: 423), HRBHRBHR (SEQ ID NO: 439), HRBHR (SEQ ID NO: 440), HRB, HRHRHR (SEQ ID NO: 426), HR, RRRRRR (SEQ ID NO: 428), RRRRRRB (SEQ ID NO: 445), RBHBH (SEQ ID NO: 431), BH, BHB, H, HB, RBH, and RBHB (SEQ ID NO: 437), e.g., RBR, R, RBRRBRR (SEQ ID NO: 420), BRR, BR, RR, HRBHRBHR (SEQ ID NO: 439), HRBHR (SEQ ID NO: 440), HRHRHR (SEQ ID NO: 426), HR, RRRRRR (SEQ ID NO: 428), RBHBH (SEQ ID NO: 431), BH, H, and RBH.

In some embodiments, the cationic domains comprise amino acid units selected from the following: R, H, B, RR, HH, BB, RH, HR, RB, BR, HB, BH, RBR, RBB, BRR, BBR, BRB, RBH, RHB, HRB, BRH, HRR, RRH, HRH, HBB, BBH, RHR, BHB, HBH, or any combination thereof.

In some embodiments, a cationic domain may also include serine, proline and/or hydroxyproline residues. In some embodiments, the cationic domains may further comprise amino acid units selected from the following: RP, PR, RPR, RRP, PRR, PRP, Hyp; R[Hyp]R, RR[Hyp], [Hyp]RR, [Hyp]R[Hyp], [Hyp][Hyp]R, R[Hyp][Hyp], SB, BS, or any combination thereof, or any combination with the above listed amino acid units.

In some embodiments, each cationic domain in the peptide may be identical or different. In some embodiments, each cationic domain in the peptide is different.

In some embodiments, each cationic domain may further comprise an N or C terminal modification. In some embodiments, the cationic domain at the C terminus comprises a C-terminal modification. In some embodiments, the cationic domain at the N terminus comprises a N-terminal modification. In some embodiments, the cationic domain at the C terminus comprises a linker group. In some embodiments, the cationic domain at the C terminus comprises a C-terminal beta-alanine. In some embodiments, the cationic domain at the N terminus is N-acetylated.

### Hydrophobic Domain

In some embodiments, the peptide comprises up to 3 hydrophobic domains, up to 2 hydrophobic domains. In some embodiments, the peptide comprises 1 hydrophobic domain, e.g., a total of 1 hydrophobic domains.

The peptide may include one or more hydrophobic domains each having a length of at least 3 amino acid residues.

In some embodiments, each hydrophobic domain has a length of between 3-6 amino acids. In some embodiments, each hydrophobic domain has a length of 5 amino acids.

In some embodiments, each hydrophobic domain may comprise nonpolar, polar, and hydrophobic amino acid residues.

Hydrophobic amino acid residues may be selected from: alanine, valine, leucine, isoleucine, phenylalanine, tyrosine, methionine, and tryptophan.

Non-polar amino acid residues may be selected from: proline, glycine, cysteine, alanine, valine, leucine, isoleucine, tryptophan, phenylalanine, methionine.

Polar amino acid residues may be selected from: serine, asparagine, hydroxyproline, histidine, arginine, threonine, tyrosine, glutamine.

In some embodiments, the hydrophobic domains do not comprise hydrophilic amino acid residues.

In some embodiments, each hydrophobic domain comprises a majority of hydrophobic amino acid residues. In some embodiments, each hydrophobic domain comprises at least 70%, at least 75%, at least 80%, at least 85%, at least 90%, at least 95%, 100% hydrophobic amino acids. In some embodiments, each hydrophobic domain consists of hydrophobic amino acid residues.

In some embodiments, each hydrophobic domain comprises a hydrophobicity of at least 0.3, at least 0.4, at least 0.5, at least 0.6, at least 0.7, at least 0.8, at least 0.8, at least 1.0, at least 1.1, at least 1.2, at least 1.3.

In some embodiments, each hydrophobic domain comprises a hydrophobicity of at least 0.3, at least 0.35, at least 0.4, at least 0.45.

In some embodiments, each hydrophobic domain comprises a hydrophobicity of at least 1.2, at least 1.25, at least 1.3, at least 1.35.

In some embodiments, each hydrophobic domain comprises a hydrophobicity of between 0.4 and 1.4

In some embodiments, each hydrophobic domain comprises of a hydrophobicity of between 0.45 and 0.48.

In some embodiments, each hydrophobic domain comprises a hydrophobicity of between 1.27 and 1.39

In some embodiments, hydrophobicity is as measured by White and Wimley: W. C. Wimley and S. H. White, “Experimentally determined hydrophobicity scale for proteins at membrane interfaces” Nature Struct Biol 3:842 (1996).

In some embodiments, each hydrophobic domain comprises at least 3, at least 4 hydrophobic amino acid residues.

In some embodiments, each hydrophobic domain comprises phenylalanine, leucine, Isoleucine, tyrosine, tryptophan, proline, and glutamine residues. In some embodiments, each hydrophobic domain consists of phenylalanine, leucine, isoleucine, tyrosine, tryptophan, proline, and/or glutamine residues.

In some embodiments, each hydrophobic domain consists of phenylalanine, leucine, isoleucine, tyrosine and/or glutamine residues.

In some embodiments, each hydrophobic domain consists of tryptophan and/or proline residues.

In some embodiments, the peptide comprises one hydrophobic domain. In some embodiments, the hydrophobic domain is located in the center of the peptide. In some embodiments, therefore, the hydrophobic domain may be known as a core hydrophobic domain. In some embodiments, the hydrophobic core domain is flanked on either side by an arm domain. In some embodiments, the arm domains may comprise one or more cationic domains and one or more further hydrophobic domains. In some embodiments, each arm domain comprises a cationic domain.

In some embodiments, the peptide comprises two arm domains flanking a hydrophobic core domain, wherein each arm domain comprises a cationic domain.

In some embodiments, the peptide consists of two cationic arm domains flanking a hydrophobic core domain.

In some embodiments, the or each hydrophobic domain comprises FQILY (SEQ ID NO: 446).

In some embodiments, the or each hydrophobic domain consists of FQILY (SEQ ID NO: 446).

In some embodiments, the hydrophobic domain comprises or consists of YQFLI (SEQ ID NO: 513), IQFLI (SEQ ID NO: 514), YRFLI (SEQ ID NO: 515), or ILFRY (SEQ ID NO: 516).

In some embodiments, each hydrophobic domain in the peptide may have the same sequence or a different sequence.

In some embodiments, a hydrophobic domain separates any two cationic domains. In some embodiments, each hydrophobic domain is flanked by cationic domains on either side thereof.

In some embodiments, no cationic domain is contiguous with another cationic domain.

In some embodiments, the peptide comprises one hydrophobic domain flanked by two cationic domains in the following arrangement:
[cationic domain]-[hydrophobic domain]-[cationic domain].

In some embodiments, the peptide consists of two cationic domains and one hydrophobic domain.

In some embodiments, the peptide consists of one hydrophobic core domain flanked by two cationic arm domains.

In some embodiments, the peptide consists of one hydrophobic core domain comprising FQILY (SEQ ID NO: 446).

In some embodiments, the peptide consists of one hydrophobic core domain that is FQILY (SEQ ID NO: 446).

### Conjugates

The peptides of the invention may be covalently linked to a therapeutic or diagnostic molecule to provide a conjugate.

The therapeutic molecule may be any molecule for treatment of a disease. The therapeutic molecule may thus optionally be selected from: a nucleic acid, peptide nucleic acid, antisense oligonucleotide (such as PNA, PMO), mRNA, gRNA (for example in the use of CRISPR/Cas9 technology), short interfering RNA, micro RNA, antagomiR, peptide, cyclic peptide, protein, pharmaceutical, drug, or nanoparticle.

In some embodiments, the therapeutic molecule is an antisense oligonucleotide (see, e.g., below and elsewhere herein). Suitably the antisense oligonucleotide comprises a phosphorodiamidate morpholino oligonucleotide (PMO). Alternatively, the oligonucleotide may be a modified PMO or any other charge-neutral oligonucleotide such as a peptide nucleic acid (PNA), a chemically modified PNA such as a gamma-PNA (Bahal, Nat. Comm. 2016 13304), oligonucleotide phosphoramidate (where the non-ridging oxygen of the phosphate is substituted by an amine or alkylamine such as those described in WO2016/028187A1), or any other partially or fully charge-neutralized oligonucleotide. In other embodiments, the antisense oligonucleotide is a are phosphorothioates, e.g., as described herein. In some embodiments, the oligonucleotides are 2′-O-alkyl oligonucleotides, e.g., 2′-O-methyl oligonucleotides and/or 2′-O-alkyl phosphorothioates, such as 2′-O-methyl phosphorothioates. In some embodiments, the oligonucleotides are PNAS.

The therapeutic antisense oligonucleotide sequence may be selected, for example, from any that are known in the art. Examples of therapeutic antisense oligonucleotides that can be included in the conjugates of the invention are provided herein.

In some embodiments, the peptides of the conjugates are covalently linked directly to a diagnostic or therapeutic molecule (e.g., an oligonucleotide).

In some embodiments, the peptides of the conjugates are linked to a diagnostic or therapeutic molecule via a linker.

The peptide of the invention may be covalently linked to an imaging molecule in order to provide a conjugate.

In some embodiments, the imaging molecule may be any molecule that enables visualization of the conjugate. In some embodiments, the imaging molecule may indicate the location of the conjugate. In some embodiments, the location of the conjugate is in vitro or in vivo. In some embodiments, there is provided a method of monitoring the location of a conjugate comprising an imaging molecule comprising: administering the conjugate to a subject and imaging the subject to locate the conjugate.

Examples of imaging molecules include detection molecules, contrast molecules, or enhancing molecules. Suitable imaging molecules may be selected from radionuclides; fluorophores; nanoparticles (such as a nanoshell); nanocages; chromogenic agents (for example an enzyme), radioisotopes, dyes, radiopaque materials, fluorescent compounds, and combinations thereof.

In some embodiments, imaging molecules are visualized using imaging techniques, these may be cellular imaging techniques or medical imaging techniques. Suitable cellular imaging techniques include image cytometry, fluorescent microscopy, phase contrast microscopy, SEM, TEM, for example. Suitable medical imaging techniques include X-ray, fluoroscopy, MRI, scintigraphy, SPECT, PET, CT, CAT, FNRI, for example.

In some cases, the imaging molecule may be regarded as a diagnostic molecule. In some embodiments, a diagnostic molecule enables the diagnosis of a disease using the conjugate. In some embodiments, diagnosis of a disease may be achieved through determining the location of the conjugate using an imaging molecule. In some embodiments, there is provided a method of diagnosis of a disease comprising administering an effective amount of a conjugate comprising an imaging molecule to a subject and monitoring the location of the conjugate.

In some embodiments, further details such as the linkage of a conjugate comprising an imaging molecule are the same as those described above in relation to a conjugate comprising an oligonucleotide.

In some embodiments, the peptide of the invention may be covalently linked to an oligonucleotide and an imaging molecule to provide a conjugate.

In some embodiments, the conjugate is capable of penetrating into cells and tissues, e.g., into the nucleus of cells, e.g., into muscle tissues.

Additional information concerning linkers that can be included in the conjugates of the invention is provided below.

### Linkers

In some embodiments, the peptides of the conjugates are linked to a diagnostic or therapeutic molecule via a linker.

Suitable linkers include, for example, a C-terminal cysteine residue that permits formation of a disulfide, thioether or thiol-maleimide linkage, a C-terminal aldehyde to form an oxime, a click reaction or formation of a morpholino linkage with a basic amino acid on the peptide or a carboxylic acid moiety on the peptide covalently conjugated to an amino group to form a carboxamide linkage.

In some embodiments, the linker is between 1-5 amino acids in length. In some embodiments, the linker may comprise any linker that is known in the art. In some embodiments, the linker is selected from any of the following sequences: G, BC, XC, C, GGC, BBC, BXC, XBC, X, XX, B, BB, BX and XB, wherein X is 6-aminohexanoic acid.

In some embodiments, the linker may be a polymer, such as for example PEG.

In some embodiments, the linker is beta-alanine.

In some embodiments, the peptide is conjugated to a therapeutic or diagnostic molecule, e.g., an oligonucleotide, through a carboxamide linkage.

The linker of the conjugate may form part of the therapeutic or diagnostic molecule (e.g., oligonucleotide) to which the peptide is attached. Alternatively, the attachment of the therapeutic or diagnostic molecule (e.g., oligonucleotide) may be directly bonded to the C-terminus of the peptide (e.g., via an amide bond). In some embodiments, in such embodiments, no linker is required.

Alternatively, the peptide may be chemically conjugated to the therapeutic or diagnostic molecule (e.g., oligonucleotide). Chemical linkage may be via a disulfide, alkenyl, alkynyl, aryl, ether, thioether, triazole, amide, carboxamide, urea, thiourea, semicarbazide, carbazide, hydrazine, oxime, phosphate, phosphoramidate, thiophosphate, boranophosphate, iminophosphates, or thiol-maleimide linkage, for example.

Optionally, cysteine may be added at the terminus of a therapeutic or diagnostic molecule (e.g., an oligonucleotide) to allow for disulfide bond formation to the peptide, or the terminus may undergo bromoacetylation for thioether conjugation to the peptide.

The linker may be a non-cationic linker, e.g., a linker including a total of 1-5 amino acids (e.g., a linker including a total of one amino acid) or an aliphatic dicarboxylic acid linker. The peptide may be attached to the linker at the side chain (e.g., to the side chain of an amino acid having a functional group amenable to covalent attachment, such as glutamic acid side chain, lysine side chain, or aspartic acid side chain).

In some embodiments, the linker is glutamic acid (e.g., via the gamma carboxyl group), beta-alanine, glycine, delta-aminovaleric acid, or gamma-aminobutyric acid.

The linker may be at the N terminus or the C terminus of the peptide.

Examples of linkers that can be used in the conjugates of the invention are noted above. Additional examples of linkers that can be used are described in WO 2020/115494 and WO 2020/030927, the contents of each of which are incorporated herein by reference.

In some embodiments, the linker is of the following structure:

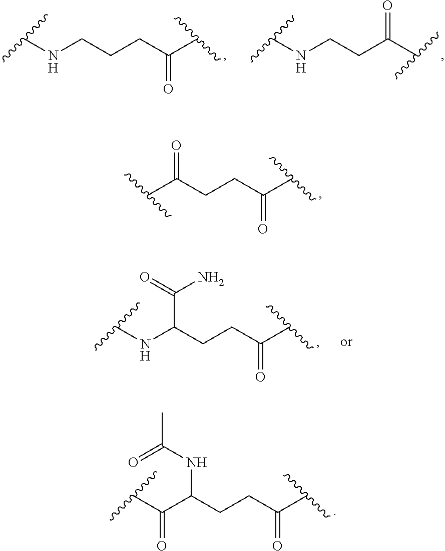 [SMILES: *NCCCC(*)=O ; *NCCC(*)=O ; *C(=O)CCC(*)=O ; *NC(CCC(*)=O)C(N)=O ; *C(=O)CCC(NC(C)=O)C(*)=O]

In some embodiments, the linker is of the following structure:

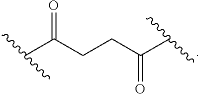 [SMILES: *C(=O)CCC(*)=O]

In some embodiments, the linker is of the following structure:

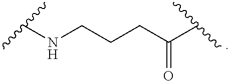 [SMILES: *NCCCC(*)=O]

In some embodiments, the linker is of the following structure:

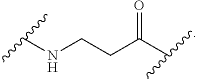 [SMILES: *NCCC(*)=O]

In some embodiments, the linker is of the following structure:

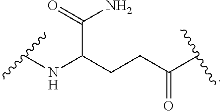 [SMILES: *NC(CCC(*)=O)C(N)=O]

In some embodiments, the linker is of the following structure:

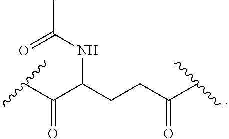 [SMILES: *C(=O)CCC(NC(C)=O)C(*)=O]

In some embodiments, the conjugate is of the following structure:

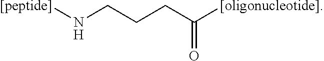

In some embodiments, the conjugate is of the following structure:

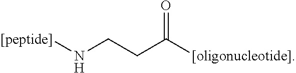

In some embodiments, the conjugate is of the following structure:

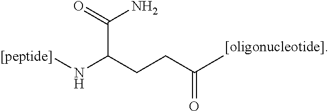

In some embodiments, the conjugate is of the following structure:

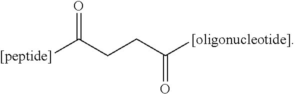

In some embodiments, the conjugate is of the following structure:

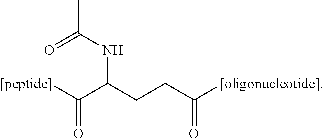

### Oligonucleotides

Oligonucleotides in the conjugates described herein include those that can be used in therapeutic approaches involving, for example, promotion of exon skipping, restoration of cryptic splicing, alteration of the levels of alternatively spliced mRNA, blocking of intermolecular interactions, or countering RNA toxicity. In some examples, the oligonucleotides promote exon skipping in DMD pre-mRNA or SMN2 pre-mRNA, and thus can be used in therapeutic approaches to muscular dystrophy (e.g., DMD or BMD) or SMA. In other examples, the oligonucleotides form heteroduplexes with the 3′-UTR of the DMPK transcript, and thus can be used in therapeutic approaches to myotonic dystrophy type 1 (e.g., DM1). Examples of oligonucleotides targeting DMD are provided below in Tables 1 (exon 45), 2 (exon 51), 3 (exon 53), and 4 (exon 44). Examples of oligonucleotides targeting the 3′-UTR of DMPK transcripts are also provided. Additionally, examples of oligonucleotides targeting the first intron of CNBP transcripts are provided. Further, examples of oligonucleotides targeting SMN2 transcripts are provided in Table 5.

In some embodiments, oligonucleotides used in the conjugates disclosed herein may be those complementary to a target site within a dystrophin transcript. Without wishing to be bound by theory, it is believed that an oligonucleotide hybridizing to certain target areas within a human dystrophin transcript may induce the skipping of any one of exons 8-55 (e.g., exon 8, exon 23, exon 44, exon 45, exon 50, exon 51, exon 52, exon 53, or exon 55) during the dystrophin pre-mRNA splicing, thereby ameliorating Duchenne muscular dystrophy. Non-limiting examples of nucleobase sequences that may be used in the oligonucleotides of the invention can be found in U.S. Pat. Nos. 8,084,601; 8,324,371; 8,461,325; 8,552,172; 9,018,368; 9,079,934; 9,243,251; 9,243,252; 9,447,417; 9,650,632; 9,970,010; 10,385,092; 10,781,450.

An oligonucleotide may include a nucleobase sequence complementary to a human dystrophin transcript and, e.g., capable of inducing exon 45 skipping. Non-limiting examples of such sequences are listed in Table 1. For example, an oligonucleotide may include, e.g., at least 12 (e.g., at least 15, at least 16, at least 17, at least 18, at least 19, or at least 20) contiguous nucleobases from any one of sequences listed in Table 1. In certain preferred embodiments, an oligonucleotide includes at least 12 (e.g., at least 15, at least 16, at least 17, at least 18, at least 19, or at least 20) contiguous nucleobases from 5′-CAAUGCCAUCCUGGAGUUCCUG-3′ (SEQ ID NO: 265) or its thymine-substitution analogue, 5′-CAATGCCATCCTGGAGTTCCTG-3′ (SEQ ID NO: 275). In certain preferred embodiments, an oligonucleotide includes a nucleobase sequence selected from the group consisting of 5′-CAAUGCCAUCCUGGAGUUCCUG-3′ (SEQ ID NO: 265) or its thymine-substitution analogue, 5′-CAATGCCATCCTGGAGTTCCTG-3′ (SEQ ID NO: 275).

TABLE 1

| # | SEQ ID NO: | Sequence |
|---|---|---|
| 1 | 253 | 5′-CCAAUGCCAUCCUGGAGUUCCUGUAAGAUA-3′ |
| 2 | 254 | 5′-GCUGCCCAAUGCCAUCCUGGAGUUCCUGUA-3′ |
| 3 | 255 | 5′-CAAUGCCAUCCUGGAGUUCCUGUAAGA-3′ |
| 4 | 256 | 5′-GCUGCCCAAUGCCAUCCUGGAGUUCCUGUAAGAUACCAA-3′ |
| 5 | 257 | 5′-GCCCAAUGCCAUCCUGGAGUUCCUGUAAGA-3′ |
| 6 | 258 | 5′-UGCCAUCCUGGAGUUCCUGUAAGAUACC-3′ |
| 7 | 259 | 5′-UGCCAUCCUGGAGUUCCUGUAAGAU-3′ |
| 8 | 260 | 5′-CAAUGCCAUCCUGGAGUUCCUGUAAGAU-3′ |
| 9 | 261 | 5′-GCCCAAUGCCAUCCUGGAGUUCCUGUAAGAU-3′ |
| 10 | 262 | 5′-UUGCCGCUGCCCAAUGCCAUCCUGGAGUUC-3′ |
| 11 | 263 | 5′-GCCCAAUGCCAUCCUGGAGUUCCUGUAA-3′ |
| 12 | 264 | 5′-GCCGCUGCCCAAUGCCAUCCUGGAGUUCCU-3′ |
| 13 | 265 | 5′-CAAUGCCAUCCUGGAGUUCCUG-3′ |
| 14 | 266 | 5′-GCCCAAUGCCAUCCUGGAGUUCCUG-3′ |
| 15 | 267 | 5′-GCUGCCCAAUGCCAUCCUGGAGUUCCUG-3′ |
| 16 | 268 | 5′-GCUGCCCAAUGCCAUCCUGGAGUUCCUGUAA-3′ |
| 17 | 269 | 5′-CAAUGCCAUCCUGGAGUUCCUGUAAGAUACC-3′ |
| 18 | 270 | 5′-TTGCCGCTGCCCAATGCCATCCTGGAGTTC-3′ |
| 19 | 271 | 5′-CAGTTTGCCGCTGCCCAATGCCATCCTGGA-3′ |
| 20 | 272 | 5′-CCAAUGCCAUCCUGGAGUUCCUGUAA-3′ |
| 21 | 273 | 5′-CCAATGCCATCCTGGAGTTCCTGTA-3′ |
| 22 | 274 | 5′-CTGACAACAGTTTGCCGCTGCCCAA-3′ |
| 23 | 275 | 5′-CAATGCCATCCTGGAGTTCCTG-3′ |
| 24 | 276 | 5′-GCTGCCCAATGCCATCCTGGAGTTCCTGTAA-3′ |

In some embodiments, one or more uracils (e.g., all uracils) in an oligonucleotide sequence shown in Table 1 are replaced with thymines. For example, an oligonucleotide sequence may be, e.g., 5′-CAAUGCCAUCCUGGAGUUCCUG-3′ (SEQ ID NO: 265). Alternatively, the oligonucleotide sequence may be, e.g., 5′-CAATGCCATCCTGGAGTTCCTG-3′ (SEQ ID NO: 275). In some embodiments, one or more thymines (e.g., all thymines) in an oligonucleotide sequence shown in Table 1 are replaced with uracils.

An oligonucleotide may include a nucleobase sequence complementary to a human dystrophin transcript and, e.g., capable of inducing exon 51 skipping. Non-limiting examples of such sequences are listed in Table 2. For example, an oligonucleotide may include, e.g., at least 12 (e.g., at least 15, at least 16, at least 17, at least 18, at least 19, or at least 20) contiguous nucleobases from any one of sequences listed in Table 2. In certain preferred embodiments, an oligonucleotide includes at least 12 (e.g., at least 15, at least 16, at least 17, at least 18, at least 19, or at least 20) contiguous nucleobases from 5′-CUCCAACAUCAAGGAAGAUGGCAUUUCUAG-3′ (SEQ ID NO: 282) or its thymine-substitution analogue, 5′-CTCCAACATCAAGGAAGATGGCATTTCTAG-3′ (SEQ ID NO: 287). In certain preferred embodiments, an oligonucleotide includes a nucleobase sequence selected from the group consisting of 5′-CUCCAACAUCAAGGAAGAUGGCAUUUCUAG-3′ (SEQ ID NO: 282) or its thymine-substitution analogue, 5′-CTCCAACATCAAGGAAGATGGCATTTCTAG-3′ (SEQ ID NO: 287).

TABLE 2

| # | SEQ ID NO: | Sequence |
|---|---|---|
| 1 | 277 | 5′-ACCAGAGUAACAGUCUGAGUAGGAGC-3′ |
| 2 | 278 | 5′-CUCAUACCUUCUGCUUGAUGAUC-3′ |
| 3 | 279 | 5′-UUCUGUCCAAGCCCGGUUGAAAUC-3′ |
| 4 | 280 | 5′-ACAUCAAGGAAGAUGGCAUUUCUAGUUUGG-3′ |
| 5 | 281 | 5′-ACAUCAAGGAAGAUGGCAUUUCUAG-3′ |
| 6 | 282 | 5′-CUCCAACAUCAAGGAAGAUGGCAUUUCUAG-3′ |
| 7 | 283 | 5′-AUCAUUUUUUCUCAUACCUUCUGCUAG-3′ |
| 8 | 284 | 5′-AUCAUUUUUUCUCAUACCUUCUGCUAGGAGCUAAAAAG-3′ |
| 9 | 285 | 5′-CACCCACCAUCACCCUCUGUG-3′ |
| 10 | 286 | 5′-AUCAUCUCGUUGAUAUCCUCAA-3′ |
| 11 | 287 | 5′-CTCCAACATCAAGGAAGATGGCATTTCTAG-3′ |

In some embodiments, one or more uracils (e.g., all uracils) in an oligonucleotide sequence shown in Table 2 are replaced with thymines. For example, an oligonucleotide sequence may be, e.g., 5′-CUCCAACAUCAAGGAAGAUGGCAUUUCUAG-3′ (SEQ ID NO: 282). Alternatively, the oligonucleotide sequence may be, e.g., 5′-CTCCAACATCAAGGAAGATGGCATTTCTAG-3′ (SEQ ID NO: 287). In some embodiments, the sequence may be 5′-GGCCAAACCTCGGCTTACCTGAAAT-3′ (SEQ ID NO: 288). In some embodiments, one or more thymines (e.g., all thymines) in an oligonucleotide sequence shown in Table 2 are replaced with uracils.

An oligonucleotide may include a nucleobase sequence complementary to a human dystrophin transcript and, e.g., capable of inducing exon 53 skipping. Non-limiting examples of such sequences are listed in Table 3. For example, an oligonucleotide may include, e.g., at least 12 (e.g., at least 15, at least 16, at least 17, at least 18, at least 19, or at least 20) contiguous nucleobases from any one of sequences listed in Table 3. In certain preferred embodiments, an oligonucleotide includes at least 12 (e.g., at least 15, at least 16, at least 17, at least 18, at least 19, or at least 20) contiguous nucleobases from 5′-CCTCCGGTTCTGAAGGTGTTCT-3′ (SEQ ID NO: 316) or 5′-GTTGCCTCCGGTTCTGAAGGTGTTC-3′ (SEQ ID NO: 325). In certain preferred embodiments, an oligonucleotide includes a nucleobase sequence selected from the group consisting of 5′-CCTCCGGTTCTGAAGGTGTTCT-3′ (SEQ ID NO: 316) and 5′-GTTGCCTCCGGTTCTGAAGGTGTTC-3′ (SEQ ID NO: 325).

TABLE 3

| | SEQ | |
|---|---|---|
| | ID | |
| # | NO: | Sequence |
| 1 | 289 | 5′-CCGGTTCTGAAGGTGTTCTTGTA-3′ |
| 2 | 290 | 5′-TCCGGTTCTGAAGGTGTTCTTGTA-3′ |
| 3 | 291 | 5′-CTCCGGTTCTGAAGGTGTTCTTGTA-3′ |
| 4 | 292 | 5′-CCTCCGGTTCTGAAGGTGTTCTTGTA-3′ |
| 5 | 293 | 5′-GCCTCCGGTTCTGAAGGTGTTCTTGTA-3′ |
| 6 | 294 | 5′-TGCCTCCGGTTCTGAAGGTGTTCTTGTA-3′ |
| 7 | 295 | 5′-CCGGTTCTGAAGGTGTTCTTGT-3′ |
| 8 | 296 | 5′-TCCGGTTCTGAAGGTGTTCTTGT-3′ |
| 9 | 297 | 5′-CTCCGGTTCTGAAGGTGTTCTTGT-3′ |
| 10 | 298 | 5′-CCTCCGGTTCTGAAGGTGTTCTTGT-3′ |
| 11 | 299 | 5′-GCCTCCGGTTCTGAAGGTGTTCTTGT-3′ |
| 12 | 300 | 5′-TGCCTCCGGTTCTGAAGGTGTTCTTGT-3′ |
| 13 | 301 | 5′-CCGGTTCTGAAGGTGTTCTTG-3′ |
| 14 | 302 | 5′-TCCGGTTCTGAAGGTGTTCTTG-3′ |
| 15 | 303 | 5′-CTCCGGTTCTGAAGGTGTTCTTG-3′ |
| 16 | 304 | 5′-CCTCCGGTTCTGAAGGTGTTCTTG-3′ |
| 17 | 305 | 5′-GCCTCCGGTTCTGAAGGTGTTCTTG-3′ |
| 18 | 306 | 5′-TGCCTCCGGTTCTGAAGGTGTTCTTG-3′ |
| 19 | 307 | 5′-CCGGTTCTGAAGGTGTTCTT-3′ |
| 20 | 308 | 5′-TCCGGTTCTGAAGGTGTTCTT-3′ |
| 21 | 309 | 5′-CTCCGGTTCTGAAGGTGTTCTT-3′ |
| 22 | 310 | 5′-CCTCCGGTTCTGAAGGTGTTCTT-3′ |
| 23 | 311 | 5′-GCCTCCGGTTCTGAAGGTGTTCTT-3′ |
| 24 | 312 | 5′-TGCCTCCGGTTCTGAAGGTGTTCTT-3′ |
| 25 | 313 | 5′-CCGGTTCTGAAGGTGTTCT-3′ |
| 26 | 314 | 5′-TCCGGTTCTGAAGGTGTTCT-3′ |
| 27 | 315 | 5′-CTCCGGTTCTGAAGGTGTTCT-3′ |
| 28 | 316 | 5′-CCTCCGGTTCTGAAGGTGTTCT-3′ |
| 29 | 317 | 5′-GCCTCCGGTTCTGAAGGTGTTCT-3′ |
| 30 | 318 | 5′-TGCCTCCGGTTCTGAAGGTGTTCT-3′ |
| 31 | 319 | 5′-CCGGTTCTGAAGGTGTTC-3′ |
| 32 | 320 | 5′-TCCGGTTCTGAAGGTGTTC-3′ |
| 33 | 321 | 5′-CTCCGGTTCTGAAGGTGTTC-3′ |
| 34 | 322 | 5′-CCTCCGGTTCTGAAGGTGTTC-3′ |
| 35 | 323 | 5′-GCCTCCGGTTCTGAAGGTGTTC-3′ |
| 36 | 324 | 5′-TGCCTCCGGTTCTGAAGGTGTTC-3′ |
| 37 | 325 | 5′-GTTGCCTCCGGTTCTGAAGGTGTTC-3′ |
| 38 | 326 | 5′-CAUUCAACUGUUGCCUCCGGUUCUGAAGGUG-3′ |
| 39 | 327 | 5′-CTGTTGCCTCCGGTTCTGAAGGTGTTCTTG-3′ |
| 40 | 328 | 5′-CAACTGTTGCCTCCGGTTCTGAAGGTGTTC-3′ |
| 41 | 329 | 5′-TTGCCTCCGGTTCTGAAGGTGTTCTTGTAC-3′ |
| 42 | 330 | 5′-CTGAAGGTGTTCTTGTACTTCATCC-3′ |
| 43 | 331 | 5′-CATTCAACTGTTGCCTCCGGTTCTGAAGGTG-3′ |

In some embodiments, one or more thymines (e.g., all thymines) in an oligonucleotide sequence shown in Table 3 are replaced with uracils. In some embodiments, one or more uracils (e.g., all uracils) in an oligonucleotide sequence shown in Table 3 are replaced with thymines.

An oligonucleotide may include a nucleobase sequence complementary to a human dystrophin transcript and, e.g., capable of inducing exon 44 skipping. Non-limiting examples of such sequences are listed in Table 4. For example, an oligonucleotide may include, e.g., at least 12 (e.g., at least 15, at least 16, at least 17, at least 18, at least 19, or at least 20) contiguous nucleobases from any one of sequences listed in Table 4.

TABLE 4

| | SEQ | |
|---|---|---|
| # | ID NO: | Sequence |
| 1 | 332 | 5′-UUUGUGUCUUUCUGAGAAAC-3′ |
| 2 | 333 | 5′-AAAGACUUACCUUAAGAUAC-3′ |
| 3 | 334 | 5′-AUCUGUCAAAUCGCCUGCAG-3′ |
| 4 | 335 | 5′-TGAAAACGCCGCCATTTCTCAACAGATCTG-3′ |
| 5 | 336 | 5′-CATAATGAAAACGCCGCCATTTCTCAACAG-3′ |
| 6 | 337 | 5′-TGTTCAGCTTCTGTTAGCCACTGATTAAAT-3′ |
| 7 | 338 | 5′-CGCCGCCATTTCTCAACAG-3′ |
| 8 | 339 | 5′-ATCTGTCAAATCGCCTGCAG-3′ |

In some embodiments, one or more uracils (e.g., all uracils) in an oligonucleotide sequence shown in Table 4 are replaced with thymines. In some embodiments, one or more thymines (e.g., all thymines) in an oligonucleotide sequence shown in Table 4 are replaced with uracils.

Alternatively, the oligonucleotide may be complementary to a r(CUG)^{exp} (e.g., the oligonucleotide may have at least 9 contiguous nucleobases complementary to a CUG repeat sequence). An oligonucleotide thus may include a nucleobase sequence selected from the group consisting of 5′-[CAG]ₙ-3′, 5′-[AGC]ₙ-3′, and 5′-[GCA]ₙ-3′, where n is an integer from 5 to 8. Non-limiting examples of the nucleobase sequences that may be included in the oligonucleotides described herein are 5′-[CAG]₅-3′ (SEQ ID NO: 340), 5′-[CAG]₆-3′ (SEQ ID NO: 341), 5′-[CAG]₇-3′ (SEQ ID NO: 342), 5′-[CAG]₈-3′ (SEQ ID NO: 343), 5′-[AGC]₅-3′ (SEQ ID NO: 344), 5′-[AGC]₆-3′ (SEQ ID NO: 345), 5′-[AGC]₇-3′ (SEQ ID NO: 346), 5′-[AGC]₈-3′ (SEQ ID NO: 347), 5′-[GCA]₅-3′ (SEQ ID NO: 348), 5′-[GCA]₆-3′ (SEQ ID NO: 349), 5′-[GCA]₇-3′ (SEQ ID NO: 350), and 5′-[GCA]₈-3′ (SEQ ID NO: 351).

Alternatively, the oligonucleotide may be complementary to a r(CCUG)^{exp} (e.g., the oligonucleotide may have at least 8 contiguous nucleobases complementary to a CCUG repeat sequence). An oligonucleotide thus may have a nucleobase sequence selected from the group consisting of 5′-[CAGG]ₙ-3′, 5′-[AGGC]ₙ-3′, 5′-[GGCA]ₙ-3′, 5′-[GCAG]ₙ-3′, wherein n is an integer from 4 to 8. Non-limiting examples of the nucleobase sequences that may be included in the oligonucleotides described herein are 5′-[CAGG]₄-3′ (SEQ ID NO: 352), 5′-[CAGG]₅-3′ (SEQ ID NO: 353), 5′-[CAGG]₆-3′ (SEQ ID NO: 354), 5′-[CAGG]₇-3′ (SEQ ID NO: 355), 5′-[CAGG]₈-3′ (SEQ ID NO: 356), 5′-[AGGC]₄-3′ (SEQ ID NO: 357), 5′-[AGGC]₅-3′ (SEQ ID NO: 358), 5′-[AGGC]₆-3′ (SEQ ID NO: 359), 5′-[AGGC]₇-3′ (SEQ ID NO: 360), 5′-[AGGC]₈-3′ (SEQ ID NO: 361), 5′-[GGCA]₄-3′ (SEQ ID NO: 362), 5′-[GGCA]₅-3′ (SEQ ID NO: 363), 5′-[GGCA]₆-3′ (SEQ ID NO: 364), 5′-[GGCA]₇-3′ (SEQ ID NO: 365), 5′-[GGCA]₆-3′ (SEQ ID NO: 366), 5′-[GCAG]₄-3′ (SEQ ID NO: 367), 5′-[GCAG]₅-3′ (SEQ ID NO: 368), 5′-[GCAG]₆-3′ (SEQ ID NO: 369), 5′-[GCAG]₇-3′ (SEQ ID NO: 370), and 5′-[GCAG]₈-3′ (SEQ ID NO: 371).

Alternatively, the oligonucleotide may be complementary to a target sequence within or proximal to intron 7 of a human SMN2 gene. An oligonucleotide thus may include a nucleobase sequence complementary to a human SMN2 gene and, e.g., capable of inducing exon 7 inclusion. Non-limiting examples of such sequences may be those at least 80% (e.g., at least 85%, at least 90%, or at least 95%) complementary to the sequences listed in Table 5. In certain embodiments, an oligonucleotide includes a nucleobase sequence that is 5′-UCACUUUCAUAAUGCUGG-3′ (SEQ ID NO: 401) or 5′-ATTCACTTTCATAATGCTGG-3′ (SEQ ID NO: 400).

TABLE 5

| # | SEQ ID NO: | Sequence |
|---|---|---|
| 1 | 372 | 5′-CCAGCAUUAUGAAAG-3′ |
| 2 | 373 | 5′-CUAGCAACAUGAAAG-3′ |
| 3 | 374 | 5′-ACAGGCCGAUGAAAG-3′ |
| 4 | 375 | 5′-UGAGAACCAUGAAAG-3′ |
| 5 | 376 | 5′-CGAGUUAGAUGAAAG-3′ |
| 6 | 377 | 5′-CCAGGGGAAUGAAAG-3′ |
| 7 | 378 | 5′-CCAGAAGGAUGAAAG-3′ |
| 8 | 379 | 5′-CGAGUCUCAUGAAAG-3′ |
| 9 | 380 | 5′-CGAGCGGUAUGAAAG-3′ |
| 10 | 381 | 5′-GGAGCGGUAUGAAAG-3′ |
| 11 | 382 | 5′-CCAGAGGUAUGAAAG-3′ |
| 12 | 383 | 5′-CCAGCGGUAUGAAAG-3′ |
| 13 | 384 | 5′-CCAGCAGUAUGAAAG-3′ |
| 14 | 378 | 5′-CCAGAAGGAUGAAAG-3′ |
| 15 | 385 | 5′-UAAGCCCUAUGAAAG-3′ |
| 16 | 386 | 5′-CUAGUUUUAUGAAAG-3′ |
| 17 | 387 | 5′-CCUUAAUUUAGAAAG-3′ |
| 18 | 388 | 5′-AAAGCAUUAUGAAAG-3′ |
| 19 | 389 | 5′-UUAGCAUUAUGAAAG-3′ |
| 20 | 390 | 5′-CGUGCAUUAUGAAAG-3′ |
| 21 | 391 | 5′-CUGUCAUUAUGAAAG-3′ |
| 22 | 392 | 5′-CUUUCAUUAUGAAAG-3′ |
| 23 | 393 | 5′-CAUUCAUUAUGAAAG-3′ |
| 24 | 394 | 5′-CCAGCAUUAUGAUUA-3′ |
| 25 | 395 | 5′-CCAGCAUUAUCUAAG-3′ |
| 26 | 396 | 5′-CCAGCAUUAUCCCAG-3′ |
| 27 | 397 | 5′-CCAGCAUUAUUUUAG-3′ |
| 28 | 398 | 5′-ATTCACTTTCATAATGCTGG-3 |
| 29 | 399 | 5′-UCACUUUCAUAAUGCUGG-3′ |

Within the above sequences, underlined nucleotides indicate mutations in the ISS-N1 wild-type sequence. All variant forms above retained the inhibitory function of ISS-N1. In some embodiments, one or more uracils (e.g., all uracils) in an oligonucleotide sequence shown in Table 5 are replaced with thymines. In some embodiments, one or more thymines (e.g., all thymines) in an oligonucleotide shown in Table 5 are replaced with uracils.

In some embodiments, an oligonucleotide includes a nucleobase sequence disclosed in Table 2 of WO 2020/028832, the disclosure of which is incorporate herein by reference in its entirety. In some embodiments, one or more uracils (e.g., all uracils) in an oligonucleotide sequence shown in Table 2 of WO 2020/028832 are replaced with thymines.

In some embodiments, an oligonucleotide includes a nucleobase sequence disclosed in Table 8 (see below). In some embodiments, one or more uracils (e.g., all uracils) in an oligonucleotide sequence shown in Table 8 are replaced with thymines. In some embodiments, all of the U's of all of the oligonucleotides shown in Table 8 are replaced with T's.

TABLE 8

| SEQ ID | | |
|---|---|---|
| NO. | Exon | Sequence |
| 1 | 8 | cuuccuggau ggcuucaau |
| 2 | 8 | guacauuaag auggacuuc |
| 3 | 8 | uaucuggaua ggugguauca |
| | | agaucuguaa |
| 4 | 8 | auguaacuga aaauguucuu cuuua |
| 5 | 8 | uggauaggug guaucaacau |
| | | cuguaagcac |
| 6 | 8 | gauagguggu aucaacaucu gu |
| 7 | 8 | uaucuggaua ggugguauca |
| | | acaucuguaa |
| 8 | 8 | aaacuuggaa gagugaugug augua |
| 9 | 8 | gcucacuugu ugaggcaaaa cuuggaa |
| 10 | 8 | gccuuggcaa cauuuccacu uccug |
| 11 | 8 | uacacacuuu accuguugag aauag |
| 12 | 8 | gauagguggu aucaacaucu guaa |
| 13 | 8 | gauagguggu aucaacaucu g |
| 14 | 8 | gauagguggu aucaacaucu guaag |
| 15 | 8 | ggugguauca acaucuguaa |
| 16 | 8 | guaucaacau cuguaagcac |
| 17 | 23 | cggcuaauuu cagagggcgc |
| | | uuucuungac n = a, u, g, or c |
| 18 | 23 | acaguggugc ugagauagua uaggcc |
| 19 | 23 | uaggccacuu uguugcucuu gc |
| 20 | 23 | uucagagggc gcuuucuuc |
| 21 | 35 | ucuucaggug caccuucugu |
| | | uucucaaucu |
| 22 | 35 | ucugugauac ucuucaggug |
| | | caccuucugu |
| 23 | 35 | ucuucugcuc gggaggugac a |
| 24 | 35 | ccaguuacua uucagaagac |
| 25 | 35 | ucuucaggug caccuucugu |
| 26 | 43 | ugcugcuguc uucuugcu |
| 27 | 43 | uuguuaacuu uuucccauu |
| 28 | 43 | uguuaacuuu uucccauugg |
| 29 | 43 | cauuuuguua acuuuuuccc |
| 30 | 43 | cuguagcuuc acccuuucc |
| 31 | 43 | gagagcuucc uguagcuuca cccuuu |
| 32 | 43 | uccuguagcu ucacccuuuc cacaggcg |
| 33 | 43 | uguguuaccu acccuugucg |
| 34 | 43 | uagacuaucu uuuauauucu guaauau |
| 35 | 43 | gagagcuucc uguagcuuca cccuuucca |
| 36 | 43 | uuccuguagc uucacccuuu |
| | | ccacaggcgu u |
| 37 | 43 | agcuuccugu agcuucaccc uuu |
| 38 | 43 | ggagagagcu uccuguagcu ucacccuuu |
| 39 | 43 | gagagcuucc uguagcuuca ccc |
| 40 | 43 | uauguguuac cuacccuugu cgguc |
| 41 | 43 | ggagagagcu uccuguagcu |
| 42 | 43 | ucacccuuuc cacaggcguu gca |
| 43 | 43 | gcugggagag agcuuccugu agcuucac |
| 44 | 43 | uguuaccuac ccuugucggu ccuuguac |
| 45 | 43 | cugcugucuu cuugcuauga auaauguc |
| 46 | 43 | ggcguugcac uuugcaaugc ugcugucu |
| 47 | 43 | uuggaaauca agcugggaga gagcuucc |
| 48 | 43 | cuacccuugu cgguccuugu acauuuug |
| 49 | 43 | gucaauccga ccugagcuuu guuguaga |
| 50 | 43 | cuugcuauga auaaugucaa uccgacc |
| 51 | 43 | uauauguguu accuacccuu gucggucc |
| 52 | 44 | uuugugucuu ucugagaaac |
| 53 | 44 | aaagacuuac cuuaagauac |
| 54 | 44 | aucugucaaa ucgccugcag |
| 55 | 44 | cgccgccauu ucucaacag |
| 56 | 44 | uuuguauuua gcauguuccc |
| 57 | 44 | ccgccauuuc ucaacag |
| 58 | 44 | uucucaggaa uuugugucuu u |
| 59 | 44 | gacaacucuu u |
| 60 | 44 | ucagcuucug uuagccacug |
| 61 | 44 | uguucagcuu cuguuagcca cuga |
| 62 | 44 | cuguucagcu ucuguuagcc acugauu |
| 63 | 44 | uucucaacag aucugucaaa ucgccugcag |
| 64 | 44 | gccacugauu aaauaucuuu auauc |
| 65 | 44 | ucuguuagcc acugauuaaa uaucuuuaua |
| 66 | 44 | gagaaacugu ucagcuucug uuagccacug |
| | | a |
| 67 | 44 | ucuuucugag aaacuguuca gcuucuguua |
| | | g |
| 68 | 44 | cagaucuguc aaaucgccug caggua |
| 69 | 44 | caacagaucu gucaaaucgc cugcag |
| 70 | 44 | aaacuguuca gcuucuguua gccacugauu |
| | | aaa |
| 71 | 44 | gaaacuguuc agcuucuguu agccacugau |
| | | u |
| 72 | 44 | aaacuguuca gcuucuguua gccacuga |
| 73 | 44 | ugagaaacug uucagcuucu guuagcca |
| 74 | 44 | uucugagaaa cuguucagcu ucuguuagcc |
| | | ac |
| 75 | 44 | uucugagaaa cuguucagcu ucuguu |
| 76 | 44 | gaucugucaa aucgccugca gguaa |
| 77 | 44 | auaaugaaaa cgccgccauu ucuca |
| 78 | 44 | aaacuguuca gcuucuguua gccac |
| 79 | 44 | uugugucuuu cugagaaacu guuca |
| 80 | 44 | ccaauucuca ggaauuugug ucuuu |
| 81 | 44 | aucgccugca gguaaaagca uaugg |
| 82 | 44 | ugaaaacgcc gccauuucuc aacagaucug |
| 83 | 44 | cauaaugaaa acgccgccau uucucaacag |
| 84 | 44 | uguucagcuu cuguuagcca cugauuaaau |
| 85 | 44 | cagaucuguc aaaucgccug cagg |
| 86 | 44 | caacagaucu gucaaaucgc cugcagg |
| 87 | 44 | cucaacagau cugucaaauc gccugcagg |
| 88 | 44 | gaucugucaa aucgccugca ggu |
| 89 | 44 | gaucugucaa aucgccugca gg |
| 90 | 44 | gaucugucaa aucgccugca g |
| 91 | 44 | cagaucuguc aaaucgccug caggu |
| 92 | 44 | cagaucuguc aaaucgccug cag |
| 93 | 44 | gugucuuucu gagaaacugu ucagc |
| 94 | 44 | gagaaacugu ucagcuucug uuagccac |
| 95 | 44 | gaaacuguuc agcuucuguu agccacug |
| 96 | 44 | cuguucagcu ucuguuagcc acug |
| 97 | 44 | aucugucaaa ucgccugcag guaaaag |
| 98 | 44 | gaucugucaa aucgccugca gguaaaagc |
| 99 | 45 | gcugaauuau uucuucccc |
| 100 | 45 | uuuuucuguc ugacagcug |
| 101 | 45 | ucuguuuuug aggauugc |
| 102 | 45 | ccaccgcaga uucaggc |
| 103 | 45 | gcccaaugcc auccugg |
| 104 | 45 | uuugcagacc uccugcc |
| 105 | 45 | caguuugccg cugccca |
| 106 | 45 | guugcauuca auguucugac |
| 107 | 45 | auuuuuccug uagaauacug g |
| 108 | 45 | gcugcccaau gcgauccugg |
| | | aguuccugua agau |
| 109 | 45 | gcugcccaau gccauccugg aguuccug |
| 110 | 45 | gcugcccaau gccauccugg |
| | | aguuccugua a |
| 111 | 45 | caaugccauc cuggaguucc |
| | | uguaagauac c |
| 112 | 45 | gcugcccaau gccauccugg |
| | | aguuccugua ag |
| 113 | 45 | ccaaugccau ccuggaguuc |
| | | cuguaagaua |
| 114 | 45 | uugccgcugc ccaaugccau |
| | | ccuggaguuc cuguaagau |
| 115 | 45 | gcugcccaau gccauccugg |
| | | aguuccugua agau |
| 116 | 45 | caaugccauc cuggaguucc uguaaga |
| 117 | 45 | caguuugccg cugcccaaug ccaucc |
| 118 | 45 | cuuccccagu ugcauucaau guuc |
| 119 | 45 | cuggcaucug uuuuugagga uug |
| 120 | 45 | uuagaucugu cgcccuaccu |
| 121 | 45 | gcugcccaau gccauccugg |
| | | aguuccugua agauaccaa |
| 122 | 45 | gcccaaugcc auccuggagu |
| | | uccuguaaga uacc |
| 123 | 45 | cauccuggag uuccuguaag auacc |
| 124 | 45 | ugccauccug gaguuccugu aagauacc |
| 125 | 45 | ugccauccug gaguuccugu aagau |
| 126 | 45 | caaugccauc cuggaguucc uguaagau |
| 127 | 45 | gcccaaugcc auccuggagu |
| | | uccuguaaga u |
| 128 | 45 | gcccaaugcc auccuggagu uccuguaa |
| 129 | 45 | gccgcugccc aaugacaucc |
| | | uggaguuccu guaa |
| 130 | 45 | gccauccugg aguuccugua agaua |
| 131 | 45 | ccaaugccau ccuggaguuc cugua |
| 132 | 45 | cugacaacag uuugccgcug cccaa |
| 133 | 45 | uuugaggauu gcugaauuau uucuu |
| 134 | 45 | caguuugccg cugcccaaug |
| | | ccauccugga |
| 135 | 45 | uugccgcugc ccaaugccau |
| | | ccuggaguuc |
| 136 | 45 | uuugccgcug cccaaugcca uccug |
| 137 | 45 | ccaaugccau ccuggaguuc cu |
| 138 | 45 | cccaaugcca uccuggaguu ccuguaaga |
| 139 | 45 | ccgcugccca augccauccu ggaguucc |
| 140 | 45 | cccaaugcca uccuggaguu |
| | | ccuguaagau |
| 141 | 45 | ccgcugccca augccauccu |
| | | ggaguuccug |
| 142 | 45 | ugcccaaugc cauccuggag |
| | | uuccuguaag |
| 143 | 45 | cccaaugcca uccuggaguu ccuguaag |
| 144 | 45 | ugcccaaugc cauccuggag uuccugua |
| 145 | 45 | caaugccauc cuggaguucc ug |
| 146 | 55 | agccucucgc ucacucaccc ugcaaagga |
| 147 | 50 | ccacucagag cucagaucuu cuaacuucc |
| 148 | 50 | cuuccacuca gagcucagau cuucuaa |
| 149 | 50 | gggauccagu auacuuacag gcucc |
| 150 | 50 | cucagagcuc agaucuu |
| 151 | 50 | ggcugcuuug cccuc |
| 152 | 50 | cucagaucuu cuaacuuccu cuuuaac |
| 153 | 50 | cucagagcuc agaucuucua acuuccucu |
| 154 | 50 | cgccuuccac ucagagcuca gaucuuc |
| 155 | 50 | ucagcucuug aaguaaacgg uuuaccg |
| 156 | 50 | uuugcccuca gcucuugaag uaaacgg |
| 157 | 50 | ggcugcuuug cccucagcuc uugaagu |
| 158 | 50 | caggagcuag gucaggcugc uuugcc |
| 159 | 50 | uccaauagug gucaguccag gagcu |
| 160 | 50 | aaagagaaug ggauccagua uacuuac |
| 161 | 50 | aaauagcuag agccaaagag aauggga |
| 162 | 50 | ggcugcuuug cccucagcuc uugaaguaaa |
| | | cgg |
| 163 | 50 | aggcugcuuu gcccucagcu cuugaaguaa |
| 164 | 50 | gucaggcugc uuugcccuca gcucuugaag |
| 165 | 50 | aggucaggcu gcuuugcccu cagcucuuga |
| 166 | 50 | cagagcucag aucuucuaac uuccu |
| 167 | 50 | cuuacaggcu ccaauagugg ucagu |
| 168 | 50 | augggaucca guauacuuac aggcu |
| 170 | 50 | aacuuccucu uuaacagaaa agcauac |
| 171 | 51 | cucauaccuu cugcuugaug auc |
| 172 | 51 | ucaaggaaga uggcauuucu |
| 173 | 51 | gaaagccagu cgguaaguuc |
| 174 | 51 | cacccaccau caccc |
| 175 | 51 | ccucugugau uuuauaacuu gau |
| 176 | 51 | ugauauccuc aaggucaccc |
| 177 | 51 | gguaccucca acaucaagga agauggcauu |
| 178 | 51 | auuucuaguu uggagauggc aguuuc |
| 179 | 51 | caucaaggaa gauggcauuu cuaguu |
| 180 | 51 | gagcagguac cuccaacauc aaggaa |
| 181 | 51 | cuccaacauc aaggaagaug gcauuucuag |
| 182 | 51 | accagaguaa cagucugagu aggag |
| 183 | 51 | caccagagua acagucugag uagga |
| 184 | 51 | ucaccagagu aacagucuga guagg |
| 185 | 51 | gucaccagag uaacagucug aguag |
| 186 | 51 | accagaguaa cagucugagu aggagc |
| 187 | 51 | uucuguccaa gcccgguuga aauc |
| 188 | 51 | acaucaagga agauggcauu ucuaguuugg |
| 189 | 51 | acaucaagga agauggcauu ucuag |
| 190 | 51 | aucauuuuuu cucauaccuu cugcu |
| 191 | 51 | cacccaccau cacccucugu g |
| 192 | 51 | aucaucucgu ugauauccuc aa |
| 193 | 51 | cuccaacauc aaggaagaug gcauuucu |
| 194 | 51 | caucaaggaa gauggcauuu cuagu |
| 195 | 52 | uugcuggucu uguuuuuc |
| 196 | 52 | ccguaaugau uguucu |
| 197 | 52 | gcuggucuug uuuuucaa |
| 198 | 52 | uggucuuguu uuucaaauuu |
| 199 | 52 | gucuuguuuu ucaaauuuug |
| 200 | 52 | cuuguuuuuc aaauuuuggg |
| 201 | 52 | uguuuuucaa auuuugggc |
| 202 | 52 | uccaacuggg gacgccucug |
| | | uuccaaaucc ugc |
| 203 | 52 | uccugcauug uugccuguaa g |
| 204 | 52 | uccaacuggg gacgccucug |
| | | uuccaaaucc |
| 205 | 52 | acuggggacg ccucuguucc a |
| 206 | 52 | ccguaaugau uguucuagcc |
| 207 | 52 | uguuaaaaaa cuuacuucga |
| 208 | 53 | cuguugccuc cgguucug |
| 209 | 53 | uuggcucugg ccuguccu |
| 210 | 53 | uucaacuguu gccuccgguu |
| | | cugaaggugu ucu |
| 211 | 53 | uacuucaucc cacugauucu gaauu |
| 212 | 53 | cugaaggugu ucuuguacuu caucc |
| 213 | 53 | cuguugccuc cgguucugaa ggugu |
| 214 | 53 | cuguugccuc cgguucugaa |
| | | gguguucuug |
| 215 | 53 | caacuguugc cuccgguucu |
| | | gaagguguuc |
| 216 | 53 | uugccuccgg uucugaaggu |
| | | guucuuguac |
| 217 | 53 | guugccuccg guucugaagg uguuc |
| 218 | 53 | cuccgguucu gaagguguuc uug |
| 219 | 53 | cuccgguucu gaagguguuc uu |
| 220 | 53 | cuccgguucu gaagguguuc u |
| 221 | 53 | cuccgguucu gaagguguuc |
| 222 | 53 | cuccgguucu gaagguguu |
| 223 | 53 | cauucaacug uugccuccgg uucug |
| 224 | 53 | cuguugccuc cgguucugaa ggug |
| 225 | 53 | cauucaacug uugccuccgg |
| | | uucugaaggu g |
| 226 | 53 | uacuaaccuu gguuucugug a |
| 227 | 53 | uguauaggga cccuccuucc augacuc |
| 228 | 53 | cuaaccuugg uuucugugau uuucu |
| 229 | 53 | gguaucuuug auacuaaccu ugguuuc |
| 230 | 53 | auucuuucaa cuagaauaaa ag |
| 231 | 53 | gauucugaau ucuuucaacu agaau |
| 232 | 53 | aucccacuga uucugaauuc |
| 233 | 53 | aaccgagacc ggacaggauu cu |
| 234 | 55 | cuguugcagu aaucuaugag |
| 235 | 55 | ugccauuguu ucaucagcuc uuu |
| 236 | 55 | ugcaguaauc uaugaguuuc |
| 237 | 55 | uccuguagga cauuggcagu |
| 238 | 55 | gagucuucua ggagccuu |
| 239 | 23 | ggccaaaccu cggcuuaccu gaaau |
| 240 | 23 | ggccaaaccu cggcuuaccu |
| 241 | 43 | aaucagcugg gagagagcuu ccuguagcu |
| 242 | 43 | ucguucuucu gucgucguaa cguuuc |
| 243 | 44 | caccgauugu cuucga |
| 244 | 44 | cccuuguacg auuuaug |
| 245 | 44 | ucuguguuua aggacucu |
| 246 | 45 | gccgcugccc aaugccaucc uggaguuccu g |
| 247 | 45 | auuagaucug ucgcccuacc ucuuuuuuc |
| 248 | 45 | ugucgcccua ccucuuuuuu cugucug |
| 249 | 45 | gcccaaugcc auccuggagu uccug |
| 250 | 50 | gagccucucg cucacucacc cugcaaagga |
| 251 | 51 | aucauuuuuu cucauaccuu cugcuaggag |
| | | cuaaaa |
| 252 | | ggaagcuaag gaagaagcug agcagg |

In some embodiments, the oligonucleotides are morpholinos, e.g., as described herein. In some embodiments, the oligonucleotides are phosphorothioates, e.g., as described herein. In some embodiments, the oligonucleotides are 2′-O-alkyl oligonucleotides, e.g., 2′-O-methyl oligonucleotides and/or 2′-O-alkyl phosphorothioates, such as 2′-O-methyl phosphorothioates. In some embodiments, the oligonucleotides are PNAs.

### Pharmaceutical Compositions

The conjugate of the invention may be formulated into a pharmaceutical composition.

In some embodiments, the pharmaceutical composition comprises a conjugate of the invention or a pharmaceutically acceptable salt thereof.

In some embodiments, the pharmaceutical composition may further comprise a pharmaceutically acceptable diluent, adjuvant, or carrier.

Suitable pharmaceutically acceptable diluents, adjuvants and carriers are well known in the art.

It should be understood that the pharmaceutical compositions of the present disclosure can further include additional known therapeutic agents, drugs, modifications of compounds into prodrugs, and the like for alleviating, mediating, preventing, and treating the diseases, disorders, and conditions described herein under medical use.

In some embodiments, the pharmaceutical composition is for use as a medicament, e.g., for use as a medicament in the same manner as described herein for the conjugate. All features described herein in relation to medical treatment using the conjugate apply to the pharmaceutical composition.

Accordingly, in a further aspect of the invention there is provided a pharmaceutical composition according to the fourth aspect for use as a medicament. In a further aspect, there is provided a method of treating a subject for a disease condition comprising administering an effective amount of a pharmaceutical composition disclosed herein.

### Medical Use

The conjugate of the invention may be used as a medicament for the treatment of a disease.

The medicament may be in the form of a pharmaceutical composition as defined above.

A method of treatment of a patient or subject in need of treatment for a disease condition is also provided, the method comprising the step of administering a therapeutically effective amount of the conjugate to the patient or subject. In some embodiments, the medical treatment requires delivery of a therapeutic molecule (e.g., an oligonucleotide) into a cell, e.g., into the nucleus of the cell.

Diseases to be treated may include any disease where improved penetration of the cell and/or nuclear membrane by a therapeutic molecule (e.g., an oligonucleotide) may lead to an improved therapeutic effect.

In some embodiments, the disease is a genetic disease.

In some embodiments, the conjugate is for use in the treatment of diseases of the neuromuscular or neurologic system.

Conjugates (e.g., those targeting dystrophin gene) comprising peptides of the invention are suitable for the treatment of Duchenne muscular dystrophy (DMD) or Becker muscular dystrophy (BMD). Conjugates (e.g., those targeting r(CUG)^{exp}) comprising peptides of the invention are suitable for the treatment of myotonic dystrophy type 1 (DM1). Conjugates (e.g., those targeting r(CCUG)^{exp}) comprising peptides of the invention are suitable for the treatment of myotonic dystrophy type 2 (DM2). Conjugates (e.g., those targeting SMN2, e.g., SMN2 intron 7) comprising peptides of the invention are suitable for the treatment of spinal muscular atrophy. Conjugates (e.g., those targeting DUX4) comprising peptides of the invention are suitable for the treatment of facioscapulohumeral muscular dystrophy (FSHD). Conjugates (e.g., those targeting PMP22) comprising peptides of the invention are suitable for the treatment of Charcot-Marie-Tooth type 1a (CMT1a). Conjugates (e.g., those targeting MFN2) comprising peptides of the invention are suitable for the treatment of Charcot-Marie-Tooth type 2a (CMT2a). Conjugates (e.g., those targeting TCF4) comprising peptides of the invention are suitable for the treatment of Fuch's corneal dystrophy (FCD). Conjugates (e.g., those targeting FXN) comprising peptides of the invention are suitable for the treatment of Friederichs's ataxia (FA).

In some embodiments, the conjugate is for use in the treatment of diseases where protein production can be restored or prevented by the modulation of splicing processes (e.g., by the promotion of exon skipping). In such embodiments, the conjugate may comprise an oligonucleotide capable of inducing splicing to restore the open reading frame of a mutated transcript and/or preventing or correcting a splicing defect and/or increasing the production of correctly spliced mRNA molecules. In some embodiments, the conjugate may comprise an oligonucleotide that reduces the levels of or ameliorates the adverse effects associated with the presence of a toxic RNA. The efficacy of treatment may be assessed using any suitable methods. In some embodiments, the efficacy of treatment may be assessed by evaluation of observation of symptoms associated with a genetic disease, e.g., a neuromuscular or neurologic disease (e.g., DMD, BMD, FSHD, DM1, DM2, CMT1a, CMT2a, FCD, FA, or SMA). In some embodiments, the evaluation is by assessment of, e.g., muscle atrophy or muscle weakness, through measures of a subject's self-reported outcomes, e.g., mobility, self-care, usual activities, pain/discomfort, and anxiety/depression, or by quality-of-life indicators, e.g., lifespan. In some embodiments, the evaluation is by assessment of functional outcomes. In some embodiments, the evaluation is by assessment of molecular markers of disease. In some embodiments, the evaluation is by assessment of levels of the relevant RNA transcripts and/or proteins. In some embodiments, the evaluation is by assessment of imaging parameters.

In some embodiments, the conjugate is for use in the treatment of a genetic disease, such as a neuromuscular or neuromuscular disease (e.g., DMD, BMD, FSHD, DM1, DM2, CMT1a, CMT2a, FCD, FA, or SMA).

In some embodiments, there is provided a conjugate according to the second aspect for use in the treatment of a genetic disease, such as a neuromuscular disease (e.g., DMD, BMD, FSHD, DM1, DM2, CMT1a, CMT2a, FCD, FA, or SMA). In some embodiments, in the case of DMD or BMD, the oligonucleotide of the conjugate is operable to increase expression of the dystrophin protein. In some embodiments, in such an embodiment, the oligonucleotide of the conjugate is operable to increase the expression of functional dystrophin protein.

In some embodiments, the conjugate increases dystrophin expression by 10%, 15%, 20%, 25%, 30%, 35%, 40%, 45%, 50%, 55%, 60%, 65%, 70%, 75%, 80%, 85%, 90%, 95%, or 100%. In some embodiments, the conjugate increases dystrophin expression by up to 50%. In some embodiments, the conjugate restores dystrophin protein expression by 10%, 15%, 20%, 25%, 30%, 35%, 40%, 45%, 50%, 55%, 60%, 65%, 70%, 75%, 80%, 85%, 90%, 95%, or 100%. In some embodiments, the conjugate restores dystrophin protein expression by up to 50%.

In some embodiments, the conjugate restores dystrophin protein function by 10%, 15%, 20%, 25%, 30%, 35%, 40%, 45%, 50%, 55%, 60%, 65%, 70%, 75%, 80%, 85%, 90%, 95%, or 100%. In some embodiments, the conjugate restores dystrophin protein function by up to 50%.

In some embodiments, the oligonucleotide of the conjugate is operable to do so by causing skipping of one or more exons during dystrophin transcription.

In some embodiments, the oligonucleotide of the conjugate causes 10%, 15%, 20%, 25%, 30%, 35%, 40%, 45%, 50%, 55%, 60%, 65%, 70%, 75%, 80%, 85%, 90%, 95%, or 100% skipping of one or more exons of the dystrophin gene. In some embodiments, the oligonucleotide of the conjugate causes up to 50% skipping of one or more exons of the dystrophin gene.

In some embodiments, the patient or subject to be treated may be any animal or human. In some embodiments, the patient or subject may be a non-human mammal. In some embodiments, the patient or subject may be male or female. In some embodiments, the subject is male.

In some embodiments, the patient or subject to be treated may be any age. In some embodiments, the patient or subject to be treated is aged between 0-40 years, e.g., 0-30, e.g., 0-25, e.g., 0-20 years of age.

In some embodiments, the conjugate is for administration to a subject systemically or locally, for example, by intramedullary, intrathecal, intraventricular, intravitreal, enteral, parenteral, intravenous, intra-arterial, intramuscular, intratumoral, subcutaneous oral or nasal routes.

In some embodiments, the conjugate is for administration to a subject intravenously.

In some embodiments, the conjugate is for administration to a subject intravenously by injection.

In some embodiments, the conjugate is for administration to a subject in a “therapeutically effective amount”, by which it is meant that the amount is sufficient to show benefit to the individual. The actual amount administered, and rate and time-course of administration, will depend on the nature and severity of the disease being treated. Decisions on dosage are within the responsibility of general practitioners and other medical doctors. Examples of the techniques and protocols can be found in Remington's Pharmaceutical Sciences, 20^{th} Edition, 2000, pub. Lippincott, Williams & Wilkins. Exemplary doses may be 1-50 mg/kg, 10-40 mg/kg, 10-35 mg/kg, 10-30 mg/kg, 10-25 mg/kg, 10-20 mg/kg, 15-40 mg/kg, 15-35 mg/kg, 15-30 mg/kg, 15-25 mg/kg, 15-20 mg/kg, 20-40 mg/kg, 20-35 mg/kg, 20-30 mg/kg, 20-25 mg/kg, 25-40 mg/kg, 25-35 mg/kg, and 25-30 mg/kg. In some embodiments, the dose is about 20 mg/kg, 25 mg/kg, or 30 mg/kg.

Advantageously, the dosage of the conjugates of the present invention may be lower, e.g., an order or magnitude lower, than the dosage required to see any effect from the therapeutic molecule (e.g., oligonucleotide) alone.

In some embodiments, treatment according to the present invention results in improvements in one or more of the following when compared to prior conjugates using currently available peptide carriers: clinical observations, at least one clinical chemistry marker (e.g., one or more of urea, creatinine, alanine transferase, aspartate transferase, alkaline phosphatase, TRIG, magnesium, potassium, calcium, sodium, chloride, KIM-1), hematology, coagulation, urinalysis (e.g., one or more of magnesium, potassium, creatinine, KIM-1), nephrotoxicity, complement activation, hepatotoxicity, histopathology, and other established markers of toxicity used in the field.

In some embodiments, after administration of the conjugates of the present invention, one or more markers of toxicity are significantly improved compared to prior conjugates using currently available peptide carriers.

In some embodiments, the level of at least one clinical chemistry marker (see, e.g., above) is improved after administration of the conjugates of the present invention when compared to prior conjugates using currently available peptide carriers.

In some embodiments, the levels of each clinical chemistry marker listed above are improved after administration of the conjugates of the present invention when compared to prior conjugates using currently available peptide carriers.

In some embodiments, the levels of the or each marker/s is significantly improved when compared to prior conjugates using currently available peptide carriers.

In some embodiments, the levels of the or each marker/s is improved by up to 5%, 10%, 15%, 20%, 25%, 30%, 35%, 40%, 45%, 50% after administration of the conjugates of the present invention when compared to prior conjugates using currently available peptide carriers.

Advantageously, the toxicity of the peptides and therefore the resulting conjugates is significantly reduced compared to prior cell-penetrating peptides and conjugates.

### Peptide Preparation

Peptides of the invention may be produced by any standard protein synthesis method (e.g., solid-phase peptide synthesis), for example, chemical synthesis or semi-chemical synthesis. Accordingly, the present invention also relates to the nucleotide sequences comprising or consisting of the DNA coding for the peptides, expression systems e.g., vectors comprising said sequences accompanied by the necessary sequences for expression and control of expression, and host cells and host organisms transformed by said expression systems.

Accordingly, a nucleic acid encoding a peptide according to the present invention is also provided.

In some embodiments, the nucleic acids may be provided in isolated or purified form.

In general, preparation techniques for peptides, oligonucleotides, and their conjugations are known in the art, see, for example, U.S. Pat. No. 9,302,014, US 2021/0299263, US 2021/0299264, and WO 2020/115494.

The following examples are meant to illustrate the invention. They are not meant to limit the invention in any way.

### EXAMPLES

### Example 1. Synthesis

### Peptide Synthesis

The peptides in Table 6 were made according to the general procedures below.

TABLE 6
Peptides Synthesized on Rink Amide Resin

| Name | Sequence | SEQ ID Nos: |
|---|---|---|
| Dpep1.9b | Ac-RBRRBRFQILYBRBRE-NH₂ | SEQ ID NO: 400 |
| Dpep3.8b | Ac-RBRRBRFQILYRBHBHE-NH₂ | SEQ ID NO: 401 |
| Dpep1.9b-del2 | Ac-RBRRBRFQILYBRE-NH₂ | SEQ ID NO: 402 |
| Dpep1.9b-del4 | Ac-RBRRBRFQILYE-NH₂ | SEQ ID NO: 512 |
| E5-E | Ac-RBRRBRRFQILYE-NH₂ | SEQ ID NO: 403 |
| G5-E | Ac-RRFQILYRBHBHE-NH₂ | SEQ ID NO: 404 |
| Dpep1.9b-enant* | Ac-rBrrBrfqilyBrBre-NH₂ | SEQ ID NO: 405 |

E and e refer to a gamma Glu linker. NH₂ at the C terminus refers to amidation of the alpha carboxyl group of E or e.
*All chiral amino acids are D-amino acids

### Solid-Phase Assembly of Resin-Bound Protected Peptides on Rink-Amide Resin.

All steps were performed at 20° C. and under inert atmosphere.
1. Resin preparation: the Fmoc-Rink Amide MBHA Resin (1.00 eq., substitution: 0.53 mmol/g) in DMF was agitated 2 hrs at 20° C.
2. Deprotection: Resin was treated with 20% piperidine for 30 min. The resin was washed with DMF (5×).
3. Coupling: The C-terminal residue was added as a solution of Fmoc-Glu(OtBu)-OH or Fmoc-D-Glu(OtBu)-OH (3.00 eq.); HBTU (2.85 eq.) and DIEA (6.00 eq.) in DMF (0) was added to the resin which was agitated 50 min. [Fmoc-AA-OH]ᵢₙᵢₜᵢₐₗ≈0.2-0.3M. The resin was then washed with DMF (5×)
4. Repeat above step 2 to 3 times for the coupling of the remaining amino acids.
5. The N-terminus Acetyl (Ac) group was added by treatment with the solution (Ac₂O/DIEA/DMF=10/5/85) for 30 min.

The peptides in Table 7 were made according to the general procedures below.

TABLE 7
Peptides Synthesized on 2-Chlorotrityl resin

Dpep3.8-Ac-RBRRBRFQILYRBHBHB-OH (SEQ ID NO: 511)
Polyarginine control (PolyR)

### Solid-Phase Assembly of Resin-Bound Protected Peptides on 2-Chlorotrityl Resin

All steps were performed at 20° C. and under inert atmosphere.
1. Resin preparation: To the chloro-2-chlorotrityl resin (1.00 eq., Sub: 1.08 mmol/g) in DCM was added Fmoc-β-Ala-OH or Fmoc-Gly-OH (0.71 eq.) and DIEA (2.84 eq.). The resin was agitated for 3 hrs. Next, MeOH (1 mL/mmol of resin) was added and agitated with the resin for 30 min. The resin was washed with DMF (5×) and filtered.
2. Deprotection: Resin was treated with 20% piperidine for 30 min. The resin was washed with DMF (5×).
3. Coupling: The second residue [Fmoc-His(Trt)-OH or Fmoc-Arg(Pbf)-OH] was added as a solution (3.00 eq.), HBTU (2.85 eq.) and DIEA (6.00 eq.) in DMF) was added to the resin and agitated 50 min ([Fmoc-AA-OH]ᵢₙᵢₜᵢₐₗ≈0.2-0.3M). The resin was then washed with DMF (5×).
4. Repeat above step 2 to 3 for the coupling of remaining amino acids.
5. The N-terminus Acetyl (Ac) group was added by treatment with a solution of (Ac₂O/DIEA/DMF=10/5/85) for 30 min.

### Peptide Cleavage and Purification:

1. Add cleavage solution (92.5% TFA/2.5% TIS/2.5% H₂O/2.5% 3-Mercaptopropionic acid) to the flask containing the side chain protected peptide and stir for 2.0 hours.
2. Precipitated peptide was washed with cold isopropyl ether.
3. Filter and collect the crude peptide as a cake.
4. Wash the filter cake with Isopropyl alcohol two more times.
5. Dry the crude peptide under vacuum 2 hours.
6. The crude peptide was purified by prep-HPLC (condition: A: 0.075% TFA in H₂O, B: ACN) and lyophilized to get the solid. The peptide was then converted to HCl salt by prep-HPLC (condition: A: 0.05% HCl in H₂O, B: ACN) to give the final product as a white solid. Final products yields >30% and final purities ≥95±2%.

### Synthesis of PMO23

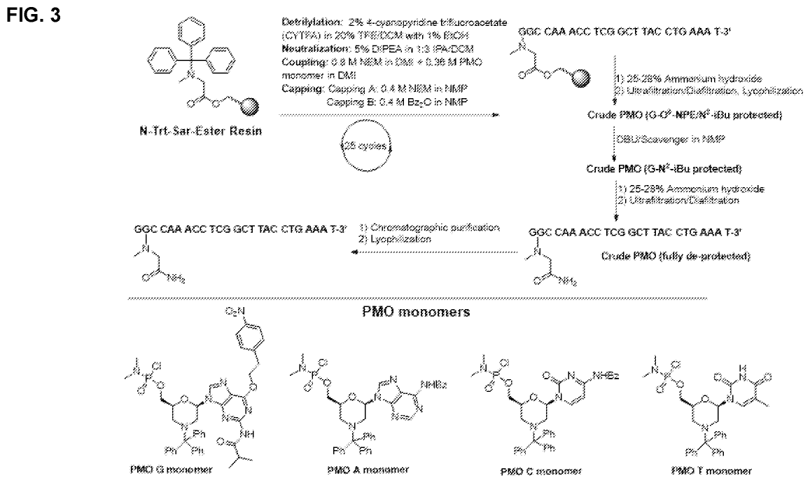

The PMO23 was synthesized according to published protocols (Ghosh ChemRxiv, 2020, 1-1; Scheme) and using commercially-available phosphorochloridate monomers (FIG. 3). 4-Nitrophenylethyl (NPE) protection was used for O⁶ of guanosine to reduce side reactions (Pon 1986). NPE was removed according to literature methods (Garcia et al., Antisense and Nucleic Acid Drug Development 11 (6): 369-378, 2001; Avino et al., Nucleosides, Nucleotides & Nucleic Acids 13 (10): 2059-2069, 1994). In short, the synthesis starts with a N-Trt-Sarcosine attached to a polystyrene via an acid-stabile ester linkage. The N-Trt group is removed with mild acid and then the resin is neutralized. Coupling of the 5′-phosphonochloramidate is accomplished with NEM as based in DMI solvent. Unreacted amino groups are capped with benzoic anhydride. The deprotection, coupling, capping cycles are repeated until the full length PMO oligomer was achieved. The oligonucleotide and the C and A protecting groups are cleaved with aqueous ammonia. The crude PMO oligomer containing the NPE protection is deblocked with DBU in the presence of thymine to prevent back alkylation of the bases with NPE (Avino 1994). Final cleavage with ammonia to remove the guanine-N2 isobutyryl groups. The crude PMO oligomer is then purified by ion reversed phase chromatography to provide a white powder after lyophilization. The final purity was 93% with an overall yield of 39%.

### Peptide PMO23 Conjugation.

| Material | | | | |
|---|---|---|---|---|
| type | Structure/Sequence | Purity | Yield/% | SEQ ID NO: |
| PMO23 | 5-GGC CAA ACC TCG GCT TAC | | N/A | SEQ ID NO: 288 |
| | CTG AAA T-3 | | | |
| Dpep1.9b | Ac-RBRRBRFQILYBRBRE-NH₂ | | N/A | SEQ ID NO: 400 |
| PPMO | Ac-RBRRBRFQILYBRBRE(PMO23)-NH₂ | 99% | 66.4% | SEQ ID NO: 400 |
| Polyarginine | | | N/A | |
| control (PolyR) | | | | |
| PolyR PPMO | | 99% | 53.2% | |

Peptide conjugation to the 3′-morpholine ring of the PMOf23 was accomplished by in situ activation of the peptide free gamma carboxylate.

### EXAMPLES

PMO23 (1.0 eq.) and Dpep1.9b (2.0 equiv) were dissolved in DMSO and coupling was achieved by adding EDCl (2.0 eq), HOBt (4.0 eq.), and DIPEA (1.0 equiv.). After 4 hours the reaction was quenched with purified water and the crude solution was purified by cation exchange chromatography and desalted by ultrafiltration/diafiltration. The final product was recovered as a white powder after lyophilization with a purity of 99% and yield of 66%.

A similar procedure for the conjugation of poly-arginine and PMO23 provided a white powder after lyophilization with a purity of 99% and yield of 53%.

### Example 2. In Vitro Efficacy

### Summary

This method describes the in vitro evaluation of exon skipping and cell viability in C2C12 myoblasts following incubation with peptides conjugated to a murine Dmd-specific PMO. Peptides are conjugated to the murine Dmd-specific PMO, PMO23, with the sequence; 5′-GGCCAAACCTCGGCTTACCTGAAAT-3′ (SEQ ID NO: 288). In this protocol, efficacy of PPMO conjugates is assessed in vitro using C2C12 myoblasts, an immortalized cell line derived from Mus musculus. The C2C12 cell line has been developed as a subclone of murine myoblast cell line originally isolated by Yaffe and Saxel (Nature, 270:725-727, 1977). C2C12 myoblasts undergo rapid differentiation to form contractile myotubes, producing characteristic muscle proteins. C2C12 myoblasts have been shown to express high levels of dystrophin (DMD) and are widely used in studies assessing ASO-mediated restoration of dystrophin levels (Xu et al., Mol. Ther., 24:564-569, 2016; Lehto et al., Nuc. Acids Res., 42:3207-3217, 2014).

Exon skipping is assessed through qPCR analysis, amplifying exons 22/23-24 for skipped/unskipped transcripts respectively. Through these quantitative readouts of skipped and unskipped transcript levels, % exon skipping in each sample can be calculated. This mDMDex23 qPCR assay, alongside the use of C2C12 myoblasts for in vitro work, enables high-throughput screening of PPMO efficacy.

### Protocol

### Myoblast Expansion

C2C12 myoblasts can be subcultured. To ensure differentiation potential is retained in C2C12 cells, under normal growth conditions confluency should not exceed 75%. The steps for C2C12 subculture are detailed below:
1. Remove growth medium and briefly rinse cell monolayer with warmed PBS.
2. Detach the cells with TrypLE. DMEM is used to neutralize TrypLE Express.
3. Count the cells and adjust cell concentration to 5.0×10³ cells per cm² culture vessel surface area.
4. Aliquot sufficient medium into tissue culture vessels and dispense the appropriate volume of cell suspension into each to complete seeding and culture the cells at 37° C., 5% CO₂.

### Myoblast-Myotube Differentiation

C2C12 myoblasts, upon reaching >90% confluency, will spontaneously begin to differentiate and form myotubes. To ensure high levels of differentiation, a specific differentiation medium is used in place of growth medium. This differentiation medium consists of DMEM supplemented with 2% horse serum and 1% antibiotic/antimycotic.
1. Cells for differentiation should be at >80% confluency, preferably being seeded 1-2 days in advance.
2. Remove growth medium and briefly rinse with pre-warmed PBS.
3. Add 1 mL of pre-warmed differentiation medium and return plate to incubator.
4. Assess daily for myotube formation and replace medium daily with fresh differentiation medium.
5. High levels of myotube formation may be observed after 7 days.

### In Vitro PPMO Treatment

Treatment of C2C12 myotubes will take place 6-8 days after differentiation begins, when high levels of differentiation are observed. Myotubes will be exposed to gymnotic delivery of PPMOs at a range of concentrations (typically between 0.1 and 20 μM) for a minimum of 48 hours.

### Cell Harvesting and RNA Extraction

Following treatment with PPMO, C2C12 myotubes will be harvested and RNA extracted, to quantify levels of DMD exon 23 skipping via qPCR. This RNA extraction will be performed using the Maxwell RSC 48 and Promega simplyRNA Tissue Kit as per the manufacturer's guidelines.

### RNA Normalization

1. Quantify extracted RNA to determine the RNA quality (260/280 ratio >1.7) and quantity (>50 ng/μL) in all samples.
   cDNA Synthesis

cDNA synthesis from normalized RNA samples will be performed using the High-Capacity cDNA Reverse Transcription Kit from ThermoFisher Scientific (4368813).
1. Prepare a master mix (RT Buffer, dNTP Mix, RT Random Primers, Multiscribe Reverse Transcriptase and Nuclease-free water) for all samples.
3. Pipette 10 μL of master mix into each reaction tube and add 10 μL of normalized RNA (50 ng/μL).
4. RNA samples are reverse transcribed as specified below.

Thermocycler conditions required for reverse transcription using the High-Capacity cDNA Reverse Transcription Kit.

| Step | Temp (° C.) | Time |
|---|---|---|
| 1 | 25 | 10 mins |
| 2 | 37 | 120 mins |
| 3 | 85 | 5 mins |
| 4 | 4 | ∞ |

qPCR mDMDex23

Gene expression can be measured by the quantitation of cDNA relative to a calibrator sample (standard, i.e., gBlocks).
1. Sequences correspond to WT cDNA (Mouse Dmd gene Ref NM007868), with the unskipped sequence running from exon20 to exon26 and the skipped from exon 20 to 26, but without exon23 (A23).
2. The calibration curve was prepared with 4 points for each calibration curve, each with a 10-fold dilution series performed in triplicate. The top point for unskipped standard is 797100 copies, and 965300 copies for skipped.
3. Prepare Primers/Probes. Primers and probes are received from IDT in IDTE pH 8.0 solution at a concentration of 100 μM. Primers are used at a final concentration of 0.5 μM, and the probes at 0.25 μM. Primer and probe sequences can be found below.

| mDMD ex23 | | |
|---|---|---|
| unskipped | Sequence 5′-3′ | SEQ ID NO: |
| Forward primer | CAGGCCATTCCTCTTTCAGG | SEQ ID NO: 406 |
| Reverse primer | GAAACTTTCCTCCCAGTTGGT | SEQ ID NO: 407 |
| Probe | /5HEX/TCAACTTCA/ZEN/GCCATCCATTTCTGTAAGGT/ | SEQ ID NO: 408 |
| | 3IABkFQ/ | |
| mDMD ex23 | | |
| skipped | Sequence 5′-3′ | SEQ ID NO: |
| Forward primer | CTGAATATGAAATAATGGAGGAGAGACTCG | SEQ ID NO: 409 |
| Reverse primer | CTTCAGCCATCCATTTCTGTAAGGT | SEQ ID NO: 410 |
| Probe | /5FAM/ATGTGATTC/ZEN/TGTAATTTCC/3IABkFQ/ | SEQ ID NO: 411 |

4. Prepare qPCR Reaction Mix. Prepare the reaction mix for each standard and sample. Standards may be run in triplicate and samples in duplicate.
5. The qPCR mDMDex23 assay was performed using the TaqMan Fast Advanced Master Mix (4444557). TaqMan® Fast Advanced Master Mix contains AmpliTaq® Fast DNA Polymerase, Uracil-N glycosylase (UNG), dNTPs (with dUTP), ROX™ dye (passive reference), and optimized buffer.
6. Dilute cDNA 1:5 (20 UL cDNA and 80 μL nuclease free water) and add 5 UL diluted cDNA per well.
7. Prepare the TaqMan master mix (TaqMan Fast Advanced Master Mix, primerspProbes, cDNA template and nuclease-free water) and transfer the appropriate volume of each reaction mixture to each well of an optical plate. Cover the plate with Optical Adhesive Film.
8. Run the qPCR on a QuantStudio6 (Thermo) under the conditions outlined below.

Temperature cycling conditions in the qPCR mDMDex23 assay.

| Step | Temp/° C. | Time | Cycles |
|---|---|---|---|
| 1 | 50 | 2 mins | 1 |
| 2 | 95 | 10 mins | 1 |
| 3 | 95 | 15 sec | 45 |
| 4 | 60 | 1 min | |

9. Post-Run Analysis: When using Applied Biosystems Real-Time PCR instruments, the Sequence Detection System (SDS) software may be used to either automatically calculate or manually set the baseline and threshold for the amplification curves. The threshold is set above the background and within the exponential growth phase of the amplification curve. Extract the raw data and analyze. Levels of respective transcripts are determined by calibration to standard curves prepared using known transcript quantities, and skipping percentages derived by ([skipped]/[skipped+unskipped])×100.

### Example 2. In Vivo Efficacy

A mouse study may be performed to assess the efficacy of the conjugates described herein. The conjugates may be evaluated for efficacy (e.g., heart and quadriceps exon skipping) and/or for toxicity (e.g., clinical signs, clinical chemistry, gross pathology, histopathology (liver and kidney), and weight profiles). The peptides used in the conjugates to be assessed as described in this example are as shown in the table below.

| Name | Sequence | SEQ ID NO: |
|---|---|---|
| Dpep1.9b | RBRRBRFQILYBRBRE | SEQ ID NO: 412 |
| Dpep3.8b | RBRRBRFQILYRBHBHE | SEQ ID NO: 413 |
| Dpep3.8 | RBRRBRFQILYRBHBHB | SEQ ID NO: 414 |
| Dpep1.9b-del2 | RBRRBRFQILYBRE | SEQ ID NO: 415 |
| Dpep1.9b-del4 | RBRRBRFQILYE | SEQ ID NO: 509 |
| E5-E | RBRRBRRFQILYE | SEQ ID NO: 416 |
| G5-E | RRFQILYRBHBHE | SEQ ID NO: 417 |
| Dpep1.9b-enant | rBrrBrfqilyBrBre* | SEQ ID NO: 418 |

E and e refer to a gamma Glu linker. The alpha carboxyl group of E or e was amidated.
*All chiral amino acid residues are D-amino acids.

The study may be performed as shown in the following table.

| | | Age | | Dose | | n |
|---|---|---|---|---|---|---|
| Group | Species | (weeks) | Agent | (mg/kg) | Administrations | (m) |
| 1 | CD1 mice | 6-8 | 0.9% saline | 0 | 1 | 5 |
| 2 | CD1 mice | 6-8 | Dpep1.9b-PMO23 | 30 | 1 | 5 |
| 3 | CD1 mice | 6-8 | Dpep1.9b-PMO23 | 60 | 1 | 5 |
| 4 | CD1 mice | 6-8 | PolyR-PMO23 | 30 | 1 | 3 |
| 5 | CD1 mice | 6-8 | PolyR-PMO23 | 60 | 1 | 3 |
| 6 | CD1 mice | 6-8 | Dpep3.8b-PMO23 | 30 | 1 | 5 |
| 7 | CD1 mice | 6-8 | Dpep3.8b-PMO23 | 60 | 1 | 5 |
| 8 | CD1 mice | 6-8 | Dpep3.8-PMO23 | 30 | 1 | 5 |
| 9 | CD1 mice | 6-8 | Dpep3.8-PMO23 | 60 | 1 | 5 |
| 10 | CD1 mice | 6-8 | Dpep1.9b-del2-PMO23 | 30 | 1 | 5 |
| 11 | CD1 mice | 6-8 | Dpep1.9b-del2-PMO23 | 60 | 1 | 5 |
| 12 | CD1 mice | 6-8 | Dpep1.9b-del4-PMO23 | 30 | 1 | 5 |
| 13 | CD1 mice | 6-8 | Dpep1.9b-del4-PMO23 | 60 | 1 | 5 |
| 14 | CD1 mice | 6-8 | E5-E-PMO23 | 30 | 1 | 5 |
| 15 | CD1 mice | 6-8 | E5-E-PMO23 | 60 | 1 | 5 |
| 16 | CD1 mice | 6-8 | G5-E-PMO23 | 30 | 1 | 5 |
| 17 | CD1 mice | 6-8 | G5-E-PMO23 | 60 | 1 | 5 |
| 18 | CD1 mice | 6-8 | Dpep1.9b-enant-PMO23 | 30 | 1 | 5 |
| 19 | CD1 mice | 6-8 | Dpep1.9b-enant-PMO23 | 60 | 1 | 5 |

The conjugates may be administered to mice intravenously as a bolus into a tail vein (over ca. 20 seconds). Serum and tissue samples may be collected 7 days post-administration at necropsy.

The safety and tolerability analysis may involve clinical observations, clinical chemistry (e.g., liver and kidney function markers), gross pathology, histopathology (e.g., liver and kidney), and/or body/tissue weight profile.

Activity analysis may be based on observations made (e.g., exon skipping) in skeletal muscle (e.g., quadriceps) and heart tissues.

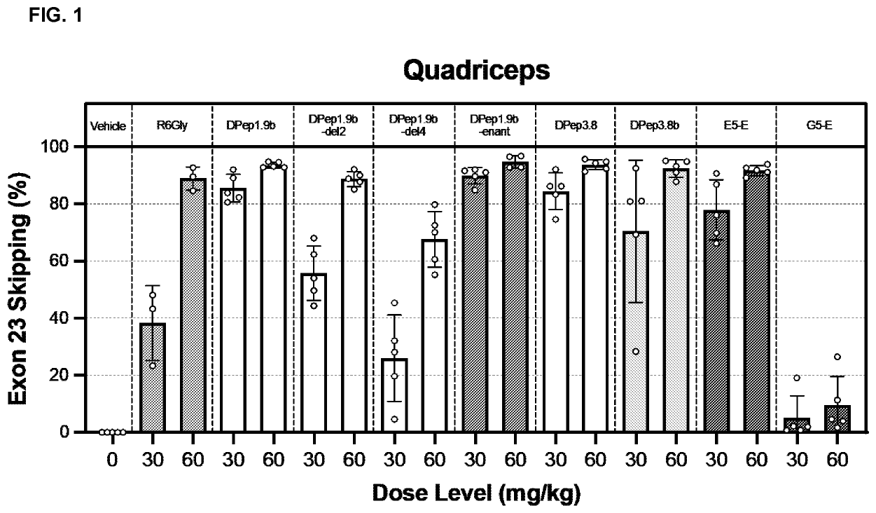

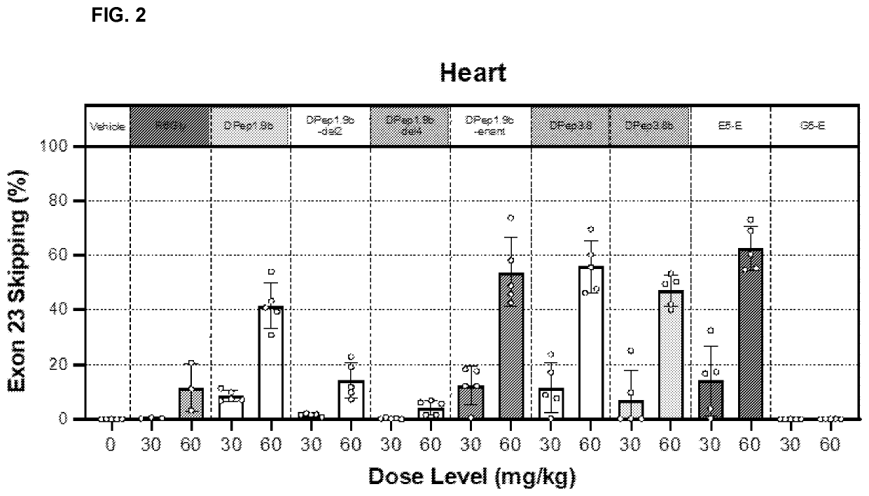

The results of exon 23 skipping in quadriceps and heart of mice receiving PPMOs are shown in FIGS. 1 and 2, respectively. Candidate PPMOs were administered to mice by bolus IV tail vein injection (over ~20 seconds) at 30 or 60 mg/kg. Dosing was performed on Day 1, and scheduled necropsy performed on Day 8. Exon skipping analysis was evaluated by qPCR. The graphs are plotted as percentage exon skipping (mean±SD, n=3-5). The results show that certain peptides present improved exon skipping in both quadriceps and heart in comparison to a polyarginine control.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A conjugate comprising a peptide and a therapeutic or diagnostic molecule covalently bonded or linked via a non-cationic linker to the peptide,
the peptide comprising a total of one hydrophobic domain and a total of one cationic domain, the cationic domain comprising at least 1 cationic amino acid residue, and the hydrophobic domain comprising at least 3 amino acid residues, provided that the peptide comprises a total of 5 to 40 (e.g., 6 to 40 or 7 to 40) amino acid residues and does not comprise any artificial amino acid residues.

2. A conjugate comprising a peptide and a therapeutic or diagnostic molecule covalently bonded or linked via a non-cationic linker to the peptide,
the peptide comprising a total of one hydrophobic domain and a total of one or two cationic domains flanking the hydrophobic domain, each cationic domain comprising at least one cationic amino acid residue, at least one of the cationic domains comprising a total of one cationic amino acid residue, and the hydrophobic domain comprising at least 3 amino acid residues, provided that the peptide comprises a total of 5 to 40 (e.g., 6 to 40 or 7 to 40) amino acid residues and does not comprise any artificial amino acid residues, wherein the amino acid residues for each cationic domain are selected from the group consisting of arginine, histidine, and beta-alanine.

3. A conjugate comprising a peptide and a therapeutic or diagnostic molecule covalently bonded or linked via a non-cationic linker to the peptide,
the peptide comprising a total of one hydrophobic domain and a total of one or two cationic domains flanking the hydrophobic domain, each cationic domain comprising at least 1 cationic amino acid residue, all cationic domains collectively comprising a total of five or fewer cationic amino acid residues, and the hydrophobic domain comprising at least 3 amino acid residues, provided that the peptide comprises a total of 5 to 40 (e.g., 6 to 40 or 7 to 40) amino acid residues and does not comprise any artificial amino acid residues, wherein the amino acid residues for each cationic domain are selected from the group consisting of arginine, histidine, and beta-alanine.

4. A conjugate comprising a peptide and a therapeutic or diagnostic molecule covalently bonded or linked via a non-cationic linker to the peptide,
the peptide comprising a total of one hydrophobic domain and a total of one or two cationic domains flanking the hydrophobic domain, each cationic domain comprising at least 1 cationic amino acid residue, at least one third of amino acid residues in the N-terminal cationic domain are histidines, and the hydrophobic domain comprising at least 3 amino acid residues, provided that the peptide comprises a total of 5 to 40 (e.g., 6 to 40 or 7 to 40) amino acid residues and does not comprise any artificial amino acid residues.

5. The conjugate of any preceding claim, wherein each cationic domain comprises at least 40%, at least 45%, or at least 50% cationic amino acids.

6. The conjugate of any preceding claim, wherein each cationic domain comprises a majority of cationic amino acids, preferably at least at least 55%, at least 60%, at least 65% at least 70%, at least 75%, at least 80%, at least 85%, at least 90%, at least 95% cationic amino acids.

7. The conjugate of claim 1 or 4-6, wherein each cationic domain comprises arginine, histidine, beta-alanine, hydroxyproline and/or serine residues, preferably wherein each cationic domain consists of arginine, histidine, beta-alanine, hydroxyproline and/or serine residues, provided at least one arginine or histidine is present.

8. The conjugate of any preceding claim, wherein each cationic domain is arginine rich and/or histidine rich, preferably each cationic domain comprises at least 40%, at least 45%, at least 50%, at least 55%, at least 60%, at least 60%, at least 65%, or least 70% arginine and/or histidine residues.

9. The conjugate of any one of claims 1 to 4, wherein at least one cationic domain is selected from the group consisting of RBRR (SEQ ID NO: 419), RBR, RB, R, RBRRBRR (SEQ ID NO: 420), RRBRR (SEQ ID NO: 426), BRR, RR, HRBHRBHRB (SEQ ID NO: 422), HRBHRB (SEQ ID NO: 423), HRB, RBRBRB (SEQ ID NO: 424), RBRB (SEQ ID NO: 425), RB, HRHRHR (SEQ ID NO: 426), HRHR (SEQ ID NO: 427), HR, RRRRRR (SEQ ID NO: 428), RBRRBR (SEQ ID NO: 429), RBHBHB (SEQ ID NO: 430), RBHBH (SEQ ID NO: 431), BHBHB (SEQ ID NO: 432), BHBH (SEQ ID NO: 433), HBHB (SEQ ID NO: 434), HBH, BHB, BH, HB, H, RBHBHE (SEQ ID NO: 435), RBHBB (SEQ ID NO: 436), RBHB (SEQ ID NO: 437), RBB, HRBRHB (SEQ ID NO: 438), HRBHRBHR (SEQ ID NO: 439), HRBHR (SEQ ID NO: 440), HRB, RBRBR (SEQ ID NO: 441), HRHRHRB (SEQ ID NO: 442), HRHRB (SEQ ID NO: 443), HRBRH (SEQ ID NO: 444), HRB, RRRRRRB (SEQ ID NO: 445), BRE, and BR.

10. The conjugate of any one of claims 1 to 4, wherein at least one cationic domain is selected from the group consisting of RBR, RBRB (SEQ ID NO: 425), RB, R, RBRRBRR (SEQ ID NO: 420), BRR, BR, RR, HRBHRBHRB (SEQ ID NO: 422), HRBHRB (SEQ ID NO: 423), HRBHRBHR (SEQ ID NO: 439), HRBHR (SEQ ID NO: 440), HRB, HRHRHR (SEQ ID NO: 426), HR, RRRRRR (SEQ ID NO: 428), RRRRRRB (SEQ ID NO: 445), RBHBH (SEQ ID NO: 431), BH, BHB, H, HB, RBH, and RBHB (SEQ ID NO: 437).

11. The conjugate of any preceding claim, wherein each hydrophobic domain has a length of between 3-6 amino acids, preferably each hydrophobic domain has a length of 5 amino acids.

12. The conjugate of any preceding claim, wherein each hydrophobic domain comprises a majority of hydrophobic amino acid residues, preferably each hydrophobic domain comprises at least 70%, at least 75%, at least 80%, at least 85%, at least 90%, at least 95%, or 100% hydrophobic amino acids.

13. The conjugate of any preceding claim, wherein each hydrophobic domain comprises phenylalanine, leucine, isoleucine, tyrosine, tryptophan, proline, and/or glutamine residues; preferably wherein each hydrophobic domain consists of phenylalanine, leucine, isoleucine, tyrosine, tryptophan, proline, and/or glutamine residues, provided at least one of phenylalanine, leucine, isoleucine, tyrosine, and tryptophan is present.

14. The conjugate of any preceding claim, wherein the hydrophobic domain is FQILY (SEQ ID NO: 446).

15. A conjugate of a peptide and a therapeutic or diagnostic molecule covalently bonded or linked via a non-cationic linker to the peptide,
the peptide being selected from the group consisting of: RBRRFQILYRBHBH (SEQ ID NO: 447), RBRFQILYRBHBH (SEQ ID NO: 448), RBFQILYRBHBH (SEQ ID NO: 449), RFQILYRBHBH (SEQ ID NO: 450), FQILYRBHBH (SEQ ID NO: 451), RBRRBRRFQILYBHBHB (SEQ ID NO: 452), RBRRBRRFQILYHBH (SEQ ID NO: 453), RBRRBRRFQILYBH (SEQ ID NO: 454), RBRRBRRFQILYH (SEQ ID NO: 455), RBRRBRRFQILY (SEQ ID NO: 456), BRRBRRFQILYRBHBH (SEQ ID NO: 457), RRBRRFQILYRBHBH (SEQ ID NO: 458), BRRFQILYRBHBH (SEQ ID NO: 459), RRFQILYRBHBH (SEQ ID NO: 460), RBRRBRRFQILYRBHB (SEQ ID NO: 461), RBRRBRRFQILYRBH (SEQ ID NO: 462), RBRRBRRFQILYRB (SEQ ID NO: 463), RBRRBRRFQILYR (SEQ ID NO: 464), HRBHRBHRBFQILYHRBRH (SEQ ID NO: 465), HRBHRBHRBFQILYHRBHRBHR (SEQ ID NO: 466), HRBHRBFQILYHRBHR (SEQ ID NO: 467), HRBFQILYHR (SEQ ID NO: 468), RBRBRBFQILYRBRBR (SEQ ID NO: 469), RBRBFQILYRBR (SEQ ID NO: 470), RBFQILYR (SEQ ID NO: 471), HRHRHRFQILYHRHRHR (SEQ ID NO: 472), HRHRFQILYHRHR (SEQ ID NO: 473), HRFQILYHR (SEQ ID NO: 474), RRRRRRFQILY (SEQ ID NO: 475), FQILYRRRRRR (SEQ ID NO: 476), RRRRRRFQILYRRRRRR (SEQ ID NO: 477), RBRRBRFQILYBR (SEQ ID NO: 478), and RBRRBRFQILY (SEQ ID NO: 479).

16. A conjugate of a peptide and a therapeutic or diagnostic molecule covalently bonded or linked via a non-cationic linker to the peptide,
The peptide being selected from the group consisting of: RBRRFQILYRBHBHB (SEQ ID NO: 481), RBRFQILYRBHBHB (SEQ ID NO: 482), RBFQILYRBHBHB (SEQ ID NO: 483), RFQILYRBHBHB (SEQ ID NO: 484), FQILYRBHBHB (SEQ ID NO: 485), RBRRBRRFQILYBHBHB (SEQ ID NO: 452), RBRRBRRFQILYHBHB (SEQ ID NO: 486), RBRRBRRFQILYBHB (SEQ ID NO: 487), RBRRBRRFQILYHB (SEQ ID NO: 488), RBRRBRRFQILYB (SEQ ID NO: 489), RBRRBRRFQILYE (SEQ ID NO: 416), RBRRBRRFQILY (SEQ ID NO: 456), BRRBRRFQILYRBHBHB (SEQ ID NO: 490), RRBRRFQILYRBHBHB (SEQ ID NO: 491), BRRFQILYRBHBHB (SEQ ID NO: 492), RRFQILYRBHBHB (SEQ ID NO: 493), RRFQILYRBHBHE (SEQ ID NO: 417), RBRRBRRFQILYRBHBB (SEQ ID NO: 494), RBRRBRRFQILYRBHB (SEQ ID NO: 461), RBRRBRRFQILYRBB (SEQ ID NO: 495), RBRRBRRFQILYRB (SEQ ID NO: 463), HRBHRBHRBFQILYHRBRHB (SEQ ID NO: 496), HRBHRBHRBFQILYHRBHRBHRB (SEQ ID NO: 497), HRBHRBFQILYHRBHRB (SEQ ID NO: 498), HRBFQILYHRB (SEQ ID NO: 499), RBRBRBFQILYRBRBRB (SEQ ID NO: 500), RBRBFQILYRBRB (SEQ ID NO: 501), RBFQILYRB (SEQ ID NO: 502), HRHRHRFQILYHRHRHRB (SEQ ID NO: 503), HRHRFQILYHRHRB (SEQ ID NO: 504), HRFQILYHRB (SEQ ID NO: 505), RRRRRRFQILYB (SEQ ID NO: 506), FQILYRRRRRRB (SEQ ID NO: 507), RRRRRRFQILYRRRRRRB (SEQ ID NO: 508), RBRRBRFQILYBRE (SEQ ID NO: 415), and RBRRBRFQILYE (SEQ ID NO: 509).

17. The conjugate of any preceding claim, wherein the peptide is bonded to the rest of the conjugate through its N-terminus.

18. The conjugate of claim 17, wherein the C-terminus of the peptide is amidated.

19. The conjugate of any one of claims 1 to 16, wherein the peptide is bonded to the rest of the conjugate through its C-terminus.

20. The conjugate of claim 19, wherein the peptide is acylated at its N-terminus.

21. The conjugate of any preceding claim, wherein the peptide is covalently bonded to the therapeutic or diagnostic molecule.

22. The conjugate of any one of claims 1 to 20, wherein the peptide is covalently linked to the therapeutic or diagnostic molecule via the non-cationic linker optionally comprising a total of one amino acid or an aliphatic dicarboxylic acid linker.

23. The conjugate of claim 22, wherein the linker is selected from the group consisting of glutamic acid, beta-alanine, glycine, delta-aminovaleric acid, and gamma-aminobutyric acid.

24. The conjugate of any one of claims 1 to 20, wherein the linker is of the following structure:

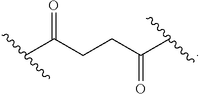 [SMILES: *C(=O)CCC(*)=O]

25. The conjugate of claim 22, wherein the linker is of the following structure:

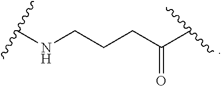 [SMILES: *NCCCC(*)=O]

26. The conjugate of claim 22, wherein the linker is of the following structure:

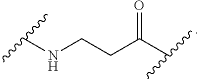 [SMILES: *NCCC(*)=O]

27. The conjugate of claim 22, wherein the linker is of the following structure:

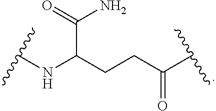 [SMILES: *NC(CCC(*)=O)C(N)=O]

28. The conjugate of claim 22, wherein the linker is of the following structure:

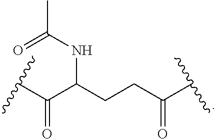 [SMILES: *C(=O)CCC(NC(C)=O)C(*)=O]

29. The conjugate of claim 22, wherein the conjugate is of the following structure:

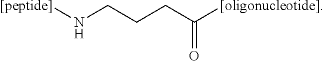

30. The conjugate of claim 22, wherein the conjugate is of the following structure:

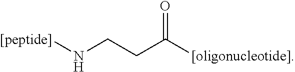

31. The conjugate of claim 22, wherein the conjugate is of the following structure:

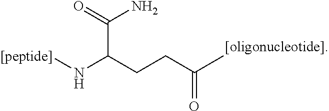

32. The conjugate of claim 22, wherein the conjugate is of the following structure:

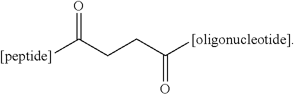

33. The conjugate of claim 22, wherein the conjugate is of the following structure:

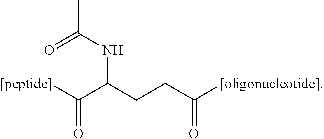

34. The conjugate of any preceding claim, wherein the therapeutic or diagnostic molecule comprises an oligonucleotide that is optionally bonded to the linker or the peptide at its 3′ terminus.

35. The conjugate of claim 34, wherein the oligonucleotide comprises a sequence that is complementary to a target sequence, the targeting of which by the oligonucleotide alters processing or recognition of the target sequence, wherein optionally the targeting can be used to treat, improve, or prevent one or more symptoms of a genetic disorder.

36. The conjugate of claim 35, wherein the genetic disorder is a neuromuscular disorder, which optionally is selected from the group consisting of muscular dystrophy (e.g., DMD, BMD, or FSHD), DM1, DM2, and SMA, and optionally the target sequence is within a transcript of the DMD, DMPK, CNBP, SMN2, or DUX4 gene.

37. The conjugate of claim 35, wherein the genetic disorder is a neurologic disorder or a disorder targeting other organ systems of the body, which optionally is CMT1a, CMT2a, FCD, or FA, and optionally the target sequence is within a transcript of the PMP22, MFN2, TF4, or FXN gene.

38. The conjugate of any one of claims 34 to 36, wherein the oligonucleotide comprises at least 12 contiguous nucleobases that are complementary to a target sequence in a dystrophin gene.

39. The conjugate of claim 38, wherein the target sequence is disposed within or proximal to any one of exons 8-55 of a transcript of the DMD gene.

40. The conjugate of claim 39, wherein the target sequence is disposed within or proximal to any one of exons 8, 23, 44, 45, 50, 51, 52, 53, or 55 of a transcript of the DMD gene.

41. The conjugate of any one of claims 35, 36, and 38 to 40, wherein the target sequence is within a transcript of the human DMD sequence.

42. The conjugate of any one of claims 38 to 41, wherein the target sequence comprises a splice site for exon 45 or is disposed within 150 nucleobases of a splice site for exon 45.

43. The conjugate of claim 42, wherein the oligonucleotide comprises at least 12 contiguous nucleobases from any one sequence in Table 1 and thymine- or uracil-substituted versions thereof.

44. The conjugate of claim 42, wherein the oligonucleotide comprises any one sequence in Table 1 or a thymine- or uracil-substituted version thereof.

45. The conjugate of claim 44, wherein the sequence in Table 1 is:
(SEQ ID NO: 276)
5′-GCTGCCCAATGCCATCCTGGAGTTCCTGTAA-3′.

46. The conjugate of claim 44, wherein the sequence in Table 1 is:
(SEQ ID NO: 275)
5′-CAATGCCATCCTGGAGTTCCTG-3′.

47. The conjugate of claim 44, wherein the sequence in Table 1 is selected from the group consisting of:
(SEQ ID NO: 270)
5′-TTGCCGCTGCCCAATGCCATCCTGGAGTTC-3′,
(SEQ ID NO: 271)
5′-CAGTTTGCCGCTGCCCAATGCCATCCTGGA-3′,
(SEQ ID NO: 272)
C5′-CAAUGCCAUCCUGGAGUUCCUGUAA-3′,
(SEQ ID NO: 273)
5′-CCAATGCCATCCTGGAGTTCCTGTA-3′,
and
(SEQ ID NO: 274)
5′-CTGACAACAGTTTGCCGCTGCCCAA-3′.

48. The conjugate of any one of claims 38 to 41, wherein the target sequence comprises a splice site for exon 51 or is disposed within 150 nucleobases of a splice site for exon 51.

49. The conjugate of claim 48, wherein the oligonucleotide comprises at least 12 contiguous nucleobases from any one sequence in Table 2 and thymine-substituted versions thereof.

50. The conjugate of claim 48, wherein the oligonucleotide comprises any one sequence in Table 2 or a thymine-substituted version thereof.

51. The conjugate of claim 50, wherein the sequence in Table 2 is:
(SEQ ID NO: 282)
5′-CUCCAACAUCAAGGAAGAUGGCAUUUCUAG-3′.

52. The conjugate of claim 50, wherein the sequence in Table 2 is:
(SEQ ID NO: 287)
5′-CTCCAACATCAAGGAAGATGGCATTTCTAG-3′.

53. The conjugate of any one of claims 38 to 41, wherein the target sequence comprises a splice site for exon 53 or is disposed within 150 nucleobases of a splice site for exon 53.

54. The conjugate of claim 53, wherein the oligonucleotide comprises at least 12 contiguous nucleobases from any one sequence in Table 3 and thymine- or uracil-substituted versions thereof.

55. The conjugate of claim 53, wherein the oligonucleotide comprises any one sequence in Table 3 or a thymine- or uracil-substituted version thereof.

56. The conjugate of claim 55, wherein the sequence in Table 3 is:
(SEQ ID NO: 316)
5′-CCTCCGGTTCTGAAGGTGTTCT-3′
or
(SEQ ID NO: 325)
5′-GTTGCCTCCGGTTCTGAAGGTGTTC-3′.

57. The conjugate of claim 55, wherein the sequence in Table 3 is selected from the group consisting of:
(SEQ ID NO: 327)
5′-CTGTTGCCTCCGGTTCTGAAGGTGTTCTTG-3′,
(SEQ ID NO: 328)
5′-CAACTGTTGCCTCCGGTTCTGAAGGTGTTC-3′,
(SEQ ID NO: 329)
5′-TTGCCTCCGGTTCTGAAGGTGTTCTTGTAC-3′,
(SEQ ID NO: 330)
5′-CTGAAGGTGTTCTTGTACTTCATCC-3′,
and
(SEQ ID NO: 331)
5′-CATTCAACTGTTGCCTCCGGTTCTGAAGGTG-3′.

58. The conjugate of any one of claims 38 to 41, wherein the target sequence comprises a splice site for exon 44 or is disposed within 150 nucleobases of a splice site for exon 44.

59. The conjugate of claim 58, wherein the oligonucleotide comprises at least 12 contiguous nucleobases from any one sequence in Table 4 and thymine- or uracil-substituted versions thereof.

60. The conjugate of claim 58, wherein the oligonucleotide comprises any one sequence in Table 4 or a thymine- or uracil-substituted version thereof.

61. The conjugate of claim 60, wherein the sequence of Table 4 is selected from the group consisting of:
(SEQ ID NO: 335)
5′-TGAAAACGCCGCCATTTCTCAACAGATCTG-3′,
(SEQ ID NO: 336)
5′-CATAATGAAAACGCCGCCATTTCTCAACAG-3′,
(SEQ ID NO: 337)
5′-TGTTCAGCTTCTGTTAGCCACTGATTAAAT-3′,
(SEQ ID NO: 338)
5′-CGCCGCCATTTCTCAACAG-3′,
and
(SEQ ID NO: 339)
5′-ATCTGTCAAATCGCCTGCAG-3′.

62. The conjugate of any one of claim 34 to 36, 38, or 39, wherein the oligonucleotide comprises the sequence 5′-GGCCAAACCTCGGCTTACCTGAAAT-3′ (SEQ ID NO: 288).

63. The conjugate of claim 42, 48, 53, or 58, wherein the splice site is an acceptor splice site.

64. The conjugate of claim 42, 48, 53, or 58, wherein the splice site is a donor splice site.

65. The conjugate of any one of claims 34 to 36, wherein the oligonucleotide comprises at least 9 contiguous nucleobases complementary to a CUG repeat sequence.

66. The conjugate of claim 65, wherein the oligonucleotide has a sequence selected from the group consisting of 5′-[CAG]ₙ-3′, 5′-[AGC]ₙ-3′, and 5′-[GCA]ₙ-3′, wherein n is an integer from 5 to 8.

67. The conjugate of claim 65, wherein the oligonucleotide has a sequence selected from the group consisting of 5′-[CAG]₅-3′ (SEQ ID NO: 340), 5′-[CAG]₆-3′ (SEQ ID NO: 341), 5′-[CAG]₇-3′ (SEQ ID NO: 342), 5′-[CAG]₈-3′ (SEQ ID NO: 343), 5′-[AGC]₅-3′ (SEQ ID NO: 344), 5′-[AGC]₆-3′ (SEQ ID NO: 345), 5′-[AGC]₇-3′ (SEQ ID NO: 346), 5′-[AGC]₈-3′ (SEQ ID NO: 347), 5′-[GCA]₅-3′ (SEQ ID NO: 348), 5′-[GCA]₆-3′ (SEQ ID NO: 349), 5′-[GCA]₇-3′ (SEQ ID NO: 350), and 5′-[GCA]₈-3′ (SEQ ID NO: 351).

68. The conjugate of any one of claims 34 to 36, wherein the oligonucleotide comprises at least 8 contiguous nucleobases complementary to a CCUG repeat sequence.

69. The conjugate of claim 68, wherein the oligonucleotide has a sequence selected from the group consisting of 5′-[CAGG]ₙ-3′, 5′-[AGGC]ₙ-3′, 5′-[GGCA]ₙ-3′, 5′-[GCAG]ₙ-3′, wherein n is an integer from 4 to 8.

70. The conjugate of claim 68, wherein the oligonucleotide has a sequence selected from the group consisting of 5′-[CAGG]₄-3′ (SEQ ID NO: 352), 5′-[CAGG]₅-3′ (SEQ ID NO: 353), 5′-[CAGG]₆-3′ (SEQ ID NO: 354), 5′-[CAGG]₇-3′ (SEQ ID NO: 355), 5′-[CAGG]₈-3′ (SEQ ID NO: 356), 5′-[AGGC]₄-3′ (SEQ ID NO: 357), 5′-[AGGC]₅-3′ (SEQ ID NO: 358), 5′-[AGGC]₆-3′ (SEQ ID NO: 359), 5′-[AGGC]₇-3′ (SEQ ID NO: 360), 5′-[AGGC]₈-3′ (SEQ ID NO: 361), 5′-[GGCA]₄-3′ (SEQ ID NO: 362), 5′-[GGCA]₅-3′ (SEQ ID NO: 363), 5′-[GGCA]₆-3′ (SEQ ID NO: 364), 5′-[GGCA]₇-3′ (SEQ ID NO: 365), 5′-[GGCA]₈-3′ (SEQ ID NO: 366), 5′-[GCAG]₄-3′ (SEQ ID NO: 367), 5′-[GCAG]₅-3′ (SEQ ID NO: 368), 5′-[GCAG]₆-3′ (SEQ ID NO: 369), 5′-[GCAG]₇-3′ (SEQ ID NO: 370), and 5′-[GCAG]₈-3′ (SEQ ID NO: 371).

71. The conjugate of any one of claims 34 to 36, wherein the oligonucleotide comprises at least 12 contiguous nucleobases that are complementary to a target sequence in a transcript of the SMN2 gene.

72. The conjugate of claim 71, wherein the target sequence is disposed in SMN2 intron 7.

73. The conjugate of claim 71, wherein the oligonucleotide comprises any one sequence in Table 5 and thymine-substituted versions thereof.

74. The conjugate of any one of claims 34 to 73, wherein the oligonucleotide comprises or consists of a sequence of a Table herein (Table 1, Table 2, Table 3, Table 4, Table 5, or Table 8), wherein optionally one or more (e.g., all) uracils are replaced with thymines or one or more (e.g., all) thymines are replaced with uracils.

75. The conjugate of any one of claims 34 to 74, wherein the oligonucleotide includes the following group as its 5′ terminus:

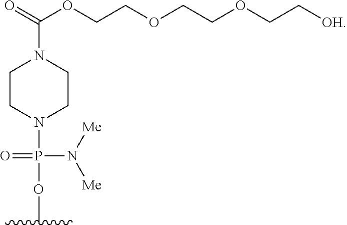 [SMILES: *OP(=O)(N(C)C)N1CCN(C(=O)OCCOCCOCCO)CC1]

76. The conjugate of any one of claims 34 to 74, wherein the oligonucleotide includes the following group as its 5′ terminus:

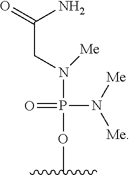 [SMILES: *OP(=O)(N(C)C)N(C)CC(N)=O]

77. The conjugate of any one of claims 34 to 76, wherein the oligonucleotide includes hydroxyl as its 5′ terminus.

78. A pharmaceutical composition comprising the conjugate of any one of claims 1 to 77 and a pharmaceutically acceptable excipient.

79. A method of treating a subject having a genetic disorder, the method comprising administering to the subject a therapeutically effective amount of a conjugate of any one of claims 1 to 77 or the pharmaceutical composition of claim 78.

80. The method of claim 79, wherein the genetic disorder is a neuromuscular disorder, which optionally is selected from the group consisting of muscular dystrophy (e.g., DMD or BMD or FSHD), DM1, DM2, and SMA, and optionally the target sequence is within a transcript of the DMD, DMPK, CNBP, SMN2, or DUX4 gene.

81. The method of claim 79, wherein the genetic disorder is a neurologic disorder or a disorder targeting other organ systems of the body, which optionally is selected from the group consisting of CMT1a, CMT2a, FCD, and FA, and optionally the target sequence is within a transcript of the PMP22, MFN2, TF4, or RXN gene.

82. The method of claim 79, wherein the method comprises administering to the subject a therapeutically effective amount of the conjugate of any one of claims 1 to 77 or the pharmaceutical composition of claim 78, wherein the conjugate is of any one of claims 1 to 36, 38 to 64, or 74 to 77 for treating DMD or BMD, the conjugate is of any one of claims 1 to 36, 65 to 67, or 74 to 77 for treating DM1, the conjugate is any one of claims 1 to 36, 68 to 70, or 74 to 77 for treating DM2, and the conjugate is of any one of claims 1 to 36 or 71 to 77 for treating SMA.

83. The method any one of claim 79, 80, or 81, wherein the subject has DMD or BMD.

84. The method of any one of claim 79, 80, or 81, wherein the subject has DM1.

85. The method of any one of claim 79, 80, or 81, wherein the subject has DM2.

86. The method of any one of claim 79, 80, or 81, wherein the subject has SMA.

87. A peptide comprising a total of one hydrophobic domain and a total of one cationic domain, the cationic domain comprising at least 1 cationic amino acid residue, and the hydrophobic domain comprising at least 3 amino acid residues, provided that the peptide comprises a total of 5 to 40 amino acid residues and does not comprise any artificial amino acid residues.

88. A peptide comprising a total of one hydrophobic domain and a total of one or two cationic domains flanking the hydrophobic domain, each cationic domain comprising at least one cationic amino acid residue, at least one of the cationic domains comprising a total of one cationic amino acid residue, and the hydrophobic domain comprising at least 3 amino acid residues, provided that the peptide comprises a total of 5 to 40 amino acid residues and does not comprise any artificial amino acid residues, wherein the amino acid residues for each cationic domain are selected from the group consisting of arginine, histidine, and beta-alanine.

89. A peptide comprising a total of one hydrophobic domain and a total of one or two cationic domains flanking the hydrophobic domain, each cationic domain comprising at least 1 cationic amino acid residue, all cationic domains collectively comprising a total of five or fewer cationic amino acid residue, and the hydrophobic domain comprising at least 3 amino acid residues, provided that the peptide comprises a total of 5 to 40 amino acid residues and does not comprise any artificial amino acid residues, wherein the amino acid residues for each cationic domain are selected from the group consisting of arginine, histidine, and beta-alanine.

90. A peptide comprising a total of one hydrophobic domain and a total of one or two cationic domains flanking the hydrophobic domain, each cationic domain comprising at least 1 cationic amino acid residue, at least one third of amino acid residues in the N-terminal cationic domain are histidines, and the hydrophobic domain comprising at least 3 amino acid residues, provided that the peptide comprises a total of 5 to 40 amino acid residues and does not comprise any artificial amino acid residues.

91. The peptide of any one of claims 87 to 90, wherein each cationic domain comprises at least 40%, at least 45%, or at least 50% cationic amino acids.

92. The peptide of any one of claims 87 to 91, wherein each cationic domain comprises a majority of cationic amino acids, preferably at least at least 55%, at least 60%, at least 65% at least 70%, at least 75%, at least 80%, at least 85%, at least 90%, at least 95% cationic amino acids.

93. The peptide of any one of claim 87 or 90-92, wherein each cationic domain comprises arginine, histidine, beta-alanine, hydroxyproline and/or serine residues, preferably wherein each cationic domain consists of arginine, histidine, beta-alanine, hydroxyproline and/or serine residues, provided at least one arginine or histidine is present.

94. The peptide of any one of claims 87 to 93, wherein each cationic domain is arginine rich and/or histidine rich, preferably each cationic domain comprises at least 40%, at least 45%, at least 50%, at least 55%, at least 60%, at least 60%, at least 65%, or least 70% arginine and/or histidine residues.

95. The peptide of any one of claims 87 to 94, wherein at least one cationic domain is selected from the group consisting of RBRR (SEQ ID NO: 419), RBR, RB, R, RBRRBRR (SEQ ID NO: 420), RRBRR (SEQ ID NO: 421), BRR, RR, HRBHRBHRB (SEQ ID NO: 422), HRBHRB (SEQ ID NO: 423), HRB, RBRBRB (SEQ ID NO: 424), RBRB (SEQ ID NO: 425), RB, HRHRHR (SEQ ID NO: 426), HRHR (SEQ ID NO: 427), HR, RRRRRR (SEQ ID NO: 428), RBRRBR (SEQ ID NO: 434), RBHBHB (SEQ ID NO: 430), RBHBH (SEQ ID NO: 431), BHBHB (SEQ ID NO: 432), BHBH (SEQ ID NO: 433), HBHB (SEQ ID NO: 434), HBH, BHB, BH, HB, H, RBHBHE (SEQ ID NO: 435), RBHBB (SEQ ID NO: 436), RBHB (SEQ ID NO: 437), RBB, HRBRHB (SEQ ID NO: 438), HRBHRBHR (SEQ ID NO: 439), HRBHR (SEQ ID NO: 440), HRB, RBRBR (SEQ ID NO: 441), HRHRHRB (SEQ ID NO: 442), HRHRB (SEQ ID NO: 443), HRBRH (SEQ ID NO: 444), HRB, RRRRRRB (SEQ ID NO: 445), BRE, and BR.

96. The peptide of any one of claims 87 to 95, wherein at least one cationic domain is selected from the group consisting of RBR, RBRB (SEQ ID NO: 425), RB, R, RBRRBRR (SEQ ID NO: 420), BRR, BR, RR, HRBHRBHRB (SEQ ID NO: 422), HRBHRB (SEQ ID NO: 423), HRBHRBHR (SEQ ID NO: 439), HRBHR (SEQ ID NO: 440), HRB, HRHRHR (SEQ ID NO: 426), HR, RRRRRR (SEQ ID NO: 428), RRRRRRB (SEQ ID NO: 445), RBHBH (SEQ ID NO: 431), BH, BHB, H, HB, RBH, and RBHB (SEQ ID NO: 437).

97. The peptide of any one of claims 87 to 96, wherein each hydrophobic domain has a length of between 3-6 amino acids, preferably each hydrophobic domain has a length of 5 amino acids.

98. The peptide of any one of claims 87 to 97, wherein each hydrophobic domain comprises a majority of hydrophobic amino acid residues, preferably each hydrophobic domain comprises at least 70%, at least 75%, at least 80%, at least 85%, at least 90%, at least 95%, or 100% hydrophobic amino acids.

99. The peptide of any one of claims 87 to 98, wherein each hydrophobic domain comprises phenylalanine, leucine, isoleucine, tyrosine, tryptophan, proline, and glutamine residues; preferably wherein each hydrophobic domain consists of phenylalanine, leucine, isoleucine, tyrosine, tryptophan, proline, and/or glutamine residues, provided at least one of phenylalanine, leucine, isoleucine, tyrosine, and tryptophan is present.

100. The peptide of any one of claims 87 to 99, wherein the peptide comprises one hydrophobic domain.

101. The peptide of any one of claims 87 to 100, wherein the hydrophobic domain is FQILY (SEQ ID NO: 446).

102. A peptide selected from the group consisting of: RBRRFQILYRBHBHB (SEQ ID NO: 481), RBRFQILYRBHBHB (SEQ ID NO: 482), RBFQILYRBHBHB (SEQ ID NO: 483), RFQILYRBHBHB (SEQ ID NO: 484), FQILYRBHBHB (SEQ ID NO: 485), RBRRBRRFQILYBHBHB (SEQ ID NO: 452), RBRRBRRFQILYHBHB (SEQ ID NO: 486), RBRRBRRFQILYBHB (SEQ ID NO: 487), RBRRBRRFQILYHB (SEQ ID NO: 488), RBRRBRRFQILYB (SEQ ID NO: 489), RBRRBRRFQILYE (SEQ ID NO: 416), RBRRBRRFQILY (SEQ ID NO: 456), BRRBRRFQILYRBHBHB (SEQ ID NO: 490), RRBRRFQILYRBHBHB (SEQ ID NO: 491), BRRFQILYRBHBHB (SEQ ID NO: 492), RRFQILYRBHBHB (SEQ ID NO: 493), RRFQILYRBHBHE (SEQ ID NO: 417), RBRRBRRFQILYRBHBB (SEQ ID NO: 494), RBRRBRRFQILYRBHB (SEQ ID NO: 461), RBRRBRRFQILYRBB (SEQ ID NO: 495), RBRRBRRFQILYRB (SEQ ID NO: 463), HRBHRBHRBFQILYHRBRHB (SEQ ID NO: 496), HRBHRBHRBFQILYHRBHRBHRB (SEQ ID NO: 497), HRBHRBFQILYHRBHRB (SEQ ID NO: 498), HRBFQILYHRB (SEQ ID NO: 499), RBRBRBFQILYRBRBRB (SEQ ID NO: 500), RBRBFQILYRBRB (SEQ ID NO: 501), RBFQILYRB (SEQ ID NO: 502), HRHRHRFQILYHRHRHRB (SEQ ID NO: 503), HRHRFQILYHRHRB (SEQ ID NO: 504), HRFQILYHRB (SEQ ID NO: 505), RRRRRRFQILYB (SEQ ID NO: 506), FQILYRRRRRRB (SEQ ID NO: 507), RRRRRRFQILYRRRRRRB (SEQ ID NO: 508), RBRRBRFQILYBRE (SEQ ID NO: 415), and RBRRBRFQILYE (SEQ ID NO: 509).

103. A peptide comprising or consisting of rBrrBrfqilyBrBr (SEQ ID NO: 510) or rBrrBrfqilyBrBre (SEQ ID NO: 513), where lower case letters indicate D-amino acids.

104. The conjugate of any one of claims 1 to 77, the pharmaceutical composition of claim 78, the method of any one of claims 79 to 86, or the peptide of any one of claims 87 to 102, wherein the peptide or the peptide of the conjugate is or comprises DPep1.9b-del2 (RBRRBRFQILYBRE) (SEQ ID NO: 415), DPep1.9b-del4 (RBRRBRFQILYE) (SEQ ID NO: 509), E5-E (RBRRBRRFQILYE) (SEQ ID NO: 416), or G5-E (RRFQILYRBHBHE) (SEQ ID NO: 417).

105. The conjugate of any one of claim 1 to 77 or 104, the pharmaceutical composition of claim 78 or 104, or the method of any one of claim 79 to 86 or 103, wherein the therapeutic or diagnostic molecule is or comprises an oligonucleotide.

106. The conjugate, pharmaceutical composition, or method of claim 105, wherein the oligonucleotide is a morpholino.

107. The conjugate, pharmaceutical composition, or method of claim 105 or 106, wherein the oligonucleotide is a morpholino with all morpholino internucleoside linkages.

108. The conjugate, pharmaceutical composition, or method of any one of claims 105 to 107, wherein the oligonucleotide is a morpholino with the following internucleoside linkages: —P(O)(NMe₂)O—).

109. The conjugate, pharmaceutical composition, or method of claim 105, wherein the oligonucleotide is a phosphorothioate (PS) oligonucleotide.

110. The conjugate, pharmaceutical composition, or method of claim 105, wherein the oligonucleotide is a 2′-O-alkyl oligoribonucleotide, e.g., a 2′-O-methyl oligoribonucleotide.

111. The conjugate, pharmaceutical composition, or method of claim 105, wherein the oligonucleotide is a 2′-O-alkyl phosphorothioate, e.g., a 2′-O-methyl phosphorothioate.

112. The conjugate, pharmaceutical composition, or method of claim 105, wherein the oligonucleotide is a peptide nucleic acid (PNA).

113. A peptide comprising or consisting of the sequence of SEQ ID NO: 415, 509, 416, 417, 418, 402, 512, 403, 404, 405, 478, 479, 456, or 460.

114. A conjugate comprising a peptide of claim 113 and a therapeutic or diagnostic molecule.

115. The conjugate of claim 114, wherein the therapeutic or diagnostic molecule comprises an oligonucleotide, such as a morpholino.

116. The conjugate of claim 115, wherein the sequence of the oligonucleotide is selected from Table 1, 2, 3, 4, 5, or 8.

117. A peptide comprising or consisting of the sequence of a peptide of claim 102, wherein the C-terminal amino acid of the peptide has been removed or removed and replaced with a linker as described herein.
